(19) United States
(12) Patent Application Publication
(10) Pub. No.: US 2026/0117232 A1
(43) Pub. Date: Apr. 30, 2026

(54) ANTISENSE OLIGONUCLEOTIDE, COMPOSITIONS AND PHARMACEUTICAL FORMULATIONS THEREOF FOR EXON SKIPPING

(71) Applicant: ASOcura Pharmaceuticals Suzhou Co., Ltd., Suzhou, Jiangsu (CN)

(72) Inventors: Yimin Hua, Miami, FL (US); Pengchao Feng, Nanjing (CN)

(73) Assignee: ASOcura Pharmaceuticals Suzhou Co., Ltd., Suzhou, Jiangsu (CN)

(21) Appl. No.: 19/116,328

(22) PCT Filed: Oct. 9, 2023
(86) PCT No.: PCT/CN2023/123640

§ 371 Date: Mar. 27, 2025

Related U.S. Application Data
(60) Provisional application No. 63/377,946, filed on Sep. 30, 2022.

(51) Int. Cl. C12N 15/113; A61K 31/712; A61K 31/7125; A61P 21/00

(52) U.S. Cl. CPC C12N 15/1135; A61K 31/712; A61K 31/7125; A61P 21/00; C12N 15/1136; C12N 15/1137; C12N 2310/11; C12N 2310/315; C12N 2310/321; C12N 2310/3233; C12N 2310/3519; C12N 2320/33; C12Y 207/10002

(57) ABSTRACT

Provided are novel antisense oligonucleotides (“ASOs”) that generate or promote exon skipping during pre-mRNA splicing, pharmaceutical compositions containing such ASOs, and their use. ASOs (e.g., bipartite ASOs) comprise a targeting sequence and a 5′-splice-site decoy sequence operably connected at the 5′ and/or 3′end of the targeting sequence. The decoy sequence may have 5, 6, 7, 8, 9, 10, 11, 12 or 13 nucleotides in length, a portion of which is complementary to the single-strand 5′end of U1 snRNA. The targeting sequence hybridizes to a targeted region, such as an exon of interest, a flanking intron sequence upstream or downstream of the exon of interest, or an intron-exon junction upstream or downstream of the exon of interest.

### PRIORITY CLAIM

This application claims the benefit of U.S. Provisional Patent Application No. 63/377,946, filed Sep. 30, 2022, which is incorporated by reference in its entirety, including drawings.

### BACKGROUND

A major tool for exon skipping is antisense oligonucleotides (ASOs). ASOs can generate or enhance exon skipping by binding to splice sites or splicing regulatory elements. The splicing process can be regulated by different cis-acting elements and trans-acting factors that influence splice site selection. ASOs either occlude the binding of RNA-binding proteins (RBPs) or snRNAs to their specific sites or disrupt secondary RNA structures, and thus promote or inhibit pre-mRNA splicing depending on what sequences they target. Matlin A, et al. Understanding alternative splicing: towards a cellular code. 2005 May. Nat. Rev. Mol. Cell Bio. 6:386-398. doi: 10.1038/nrm1645. Aartsma-Rus A, et al. Exonic sequences provide better targets for antisense oligonucleotides than splice site sequences in the modulation of Duchenne muscular dystrophy splicing. Oligonucleotides. 2010 April; 20 (2): 69-77. doi: 10.1089/oli. 2009.0215.

An example of human genetic diseases associated with mutations that disrupt the reading frame is Duchenne muscular dystrophy (DMD), a progressive form of muscular dystrophy which causes progressive weakness and loss (atrophy) of skeletal and heart muscles. Muscle weakness worsens with age and progresses to the arms, legs and trunk. Most children with DMD use a wheelchair full time by age 13. Heart and respiratory muscle problems begin in the teen years and lead to serious, life-threatening complications.

DMD is caused by mutations in the DMD gene. The DMD gene codes for the protein dystrophin. Dystrophin is mainly made in skeletal and heart muscle cells, but a small amount is also made in neurons in specific parts of the brain. DMD is inherited in an X-linked recessive pattern. Becker muscular dystrophy (BMD) is also caused by mutations in the DMD gene. People with BMD have less severe symptoms than DMD.

### SUMMARY

Certain aspects of the present technology generally relate to novel antisense oligonucleotides (ASOs), methods of using such ASOs to generate or promote skipping of an exon of interest during pre-mRNA splicing, compositions (e.g., pharmaceutical compositions) that comprise such ASOs, and methods of using such compositions to treat diseases and/or complications thereof. The ASO-mediated exon skipping may be an approach to manipulate expression of a gene of interest. The expression of the gene of interest may be manipulated by ASOs to induce exon skipping, leading to, e.g., without limitation, correcting a reading frame caused by frameshift mutations, skipping a toxic part of the gene, silencing the gene, creating a dominant negative isoform, or changing the structure and function of a gene. Exon skipping of the gene of interest may be beneficial in treating the diseases and/or complications thereof, although the gene of interest may or may not be a cause of the diseases. The diseases may be genetic or non-genetic disease (e.g., some non-genetic cancer, metabolic diseases or infectious diseases) as disclosed herein. The genetic diseases may or may not be associated with a splicing defect.

In some aspects, the present technology provides an oligonucleotide, which may also be referred to as a bipartite ASO, comprising or consisting of a targeting sequence (also referred to as “ASO targeting sequence”) and a 5′-splice-site decoy sequence (also referred to as the “ASO decoy,” the “ASO decoy sequence,” the “decoy sequence” or the “decoy”) operably connected at the 5′ and/or 3′ end of the targeting sequence. The decoy sequence may comprise a nucleotide sequence of 5, 6, 7, 8, 9, 10, or 11 nucleotides that is complementary to part or all of the single-strand 5′ end of U1 snRNA; and the targeting sequence hybridizes to a sequence selected from the group consisting of the exon of interest, a flanking intron sequence upstream of the exon of interest, a flanking intron sequence downstream of the exon of interest, an intron-exon junction upstream of the exon of interest, and an intron-exon junction downstream of the exon of interest. The decoy sequence may resemble an optimal 5′splice site.

In certain embodiments of the ASOs comprising the same targeting sequence, the ASOs comprising a decoy sequence may have higher exon skipping effects and/or efficiencies than the ASO consisting of the targeting sequence. The exon skipping effects and/or efficiencies may be quantified by the percentage of exclusion of the exon of interest (% excl, also referred to as the exon skipping percentage) in total transcripts of each gene. In certain embodiments, the improvement as quantified by increase of % excl may be over about 14 fold, over about 15 fold, over about 16 fold, or over about 17 fold.

In some embodiments, the ASO decoy sequence comprises or consists of a nucleotide sequence selected from the group consisting of SEQ ID Nos. 1 to 23 and 347 to 353.

Examples of the exon of interest include, without limitation, exon 7 of the endogenous SMN1 and SMN2 genes, exons 45, 51 and 53 of the endogenous DMD gene, exon 17 of the endogenous APP gene, exon 41 of the endogenous CEP290 gene, exon 19 of the endogenous HER2 gene, exon 10 of the SCA3 gene, exon 10 of the endogenous PKM gene, and exon 6 of the endogenous MDM4 gene.

In some embodiments, the ASO targeting sequence comprises or consists of a nucleotide sequence selected from the group consisting of the nucleotide sequences of SEQ ID Nos. 25, 29, 33, 37, 41, 45, 49, 75, 92, 124, 156, 188, 220, 252, 284, and 316.

The targeting sequence may be linked to the decoy sequence directly or via a linker (e.g., without limitation, having a length of 1, 2, 3, 4, or 5 nucleotides).

In some embodiments, the ASO (e.g., bipartite ASO) comprises or consists of a nucleotide sequence selected from the group consisting of SEQ ID Nos. 26 to 27, 30 to 31, 34 to 35, 38 to 39, 42 to 43, 46 to 47, 50 to 73, 76 to 90, 93 to 122, 125 to 154, 157 to 186, 189 to 218, 221 to 250, 253 to 282, 285 to 314, 317 to 346, and 354 to 356.

In some embodiments, the ASO (e.g., bipartite ASO) comprises at least one nucleotide analog. Examples of the nucleotide analog include, without limitation, 2′-O-methoxyethyl (MOE)-modified oligonucleotides with phosphodiester or phosphorothioate backbone, and phosphorodamidate morpholino oligomers.

In some aspects, the present technology provides a composition comprising the ASO (e.g., bipartite ASO) disclosed herein and a pharmaceutically acceptable carrier. In certain embodiments, the composition is a pharmaceutical formulation or composition.

In some aspects, the present technology provides a vector encoding the ASO (e.g., bipartite ASO) disclosed herein.

In some aspects, the present technology provides a method of generating or promoting exon skipping of an exon of interest during pre-mRNA splicing comprising contacting a pre-mRNA in a cell or a subject with the ASO (e.g., bipartite ASO) disclosed herein, the composition (e.g., pharmaceutical composition) comprising the ASO (e.g., bipartite ASO) as disclosed herein, and/or the vector encoding the ASO (e.g., bipartite ASO) as disclosed herein. In some embodiments, the method further comprises delivering to the cell or administering to the subject the ASO (e.g., bipartite ASO) disclosed herein. The ASO-mediated exon skipping may be an approach to manipulate expression of a gene of interest. The expression of the gene of interest may be manipulated by ASOs to inhibit splicing of an exon, intron or specific splice site of a gene of interest, leading to, e.g., without limitation, restoring the reading frame of a defective gene of interest, generating a different isoform of a gene of interest (e.g., a dominant negative isoform), skipping a toxic part of the gene, silencing the gene, and/or changing the structure and function of a gene.

In some aspects, the present technology provides a method of improving exon skipping efficacy and/or efficiency of a targeting sequence comprising obtaining one or more ASOs (e.g., bipartite ASOs) comprising the targeting sequence and a decoy sequence operably connected at the 5′ end and/or 3′ end of the targeting sequence, the decoy sequence comprising a nucleotide sequence of 5, 6, 7, 8, 9, 10, or 11 nucleotides that is complementary to part or all of the single-strand 5′ end of U1 snRNA. The decoy sequence may resemble an optimal 5′splice site. In certain embodiments, the method further comprises screening the one or more ASOs (e.g., bipartite ASOs) according to their exon skipping efficacies and/or efficiencies of the exon of interest. The exon skipping effects and/or efficiencies may be quantified by the percentage of exclusion of the exon of interest (% excl) in total transcripts of each gene. The targeting sequence may be capable of hybridizing to a sequence selected from the group consisting of an exon of interest, a flanking intron sequence upstream of the exon of interest, a flanking intron sequence downstream of the exon of interest, an intron-exon junction upstream of the exon of interest, and an intron-exon junction downstream of the exon of interest in a cell or subject.

In some aspects, the present technology provides a method of treating a disease and/or complications thereof in a subject comprising administering to the subject the ASO (e.g., bipartite ASO) disclosed herein, the composition (e.g., pharmaceutical composition) comprising the ASO (e.g., bipartite ASO) as disclosed herein, and/or the vector encoding the ASO (e.g., bipartite ASO) as disclosed herein. The ASO (e.g., bipartite ASO) may generate or promote skipping of the exon of interest during pre-mRNA splicing. The ASO-mediated exon skipping may be an approach to manipulate expression of the gene of interest. The expression of the gene of interest may be manipulated by ASOs to inhibit splicing of an exon, intron or a specific splice site of a gene of interest, leading to, e.g., without limitation, restoring the reading frame of a defective gene of interest, generating a different isoform of a gene of interest (e.g., a dominant negative isoform), skipping a toxic part of the gene, silencing the gene, and/or changing the structure and function of a gene. The manipulation of expression of the gene of interest by ASOs may be beneficial in treating the diseases and/or complications thereof, although the gene of interest may or may not be a cause of the diseases. The diseases may be genetic or non-genetic disease (e.g., some non-genetic cancer, metabolic diseases or infectious diseases) as disclosed herein. The genetic diseases may or may not be associated with mutations related to splicing defect.

Examples of the disease and/or complications thereof include, without limitation, diseases and/or complications thereof that may benefit from exon skipping on one or more genes selected from the group consisting of SMN1, SMN2, DMD, APP, CEP290, HER2, SCA3, PKM, and MDM4 genes. For example, without limitation, the disease and/or complications thereof may be treated by exon skipping of one or more exons selected from the group consisting of exon 7 of the endogenous SMN1 and SMN2 genes, exons 45, 51 and 53 of the endogenous DMD gene, exon 17 of the endogenous APP gene, exon 41 of the endogenous CEP290 gene, exon 19 of the endogenous HER2 gene, exon 10 of the SCA3 gene, exon 10 of the endogenous PKM gene, and exon 6 of the endogenous MDM4 gene. Examples of the disease may include, without limitation, Duchenne muscular dystrophy (DMD), Alzheimer's disease, Joubert Syndrome, spinocerebellar ataxia 3 (SCA3), cancer, e.g., without limitation, breast cancer, HER2-positive biliary tract, colorectal, non-small-cell lung, bladder cancers, prostate cancer, lung cancer, cervix cancer, kidney cancer, papillary thyroid cancer, colon cancer, colorectal cancer, gliomas, ovarian cancer, gastric cancer, hepatoblastoma, fibrolamellar, hepatocellular, carcinoma, soft tissue sarcoma, osteosarcoma, chronic lymphocytic, leukemia, acute myeloid leukemia, mantle cell lymphoma, Pediatric Burkitt, lymphoma, salivary gland cancer, liver cancer, and melanoma. In certain embodiments, the ASO (e.g., bipartite ASO), the composition (e.g., pharmaceutical composition) comprising the ASO (e.g., bipartite ASO), and/or the vector encoding the ASO (e.g., bipartite ASO) are administered at a therapeutically effective amount.

In some aspects, the present technology provides a use of the ASO (e.g., bipartite ASO) disclosed herein, the composition (e.g., pharmaceutical composition) comprising the ASO (e.g., bipartite ASO) as disclosed herein, and/or the vector encoding the ASO (e.g., bipartite ASO) as disclosed herein to generate or promote exon skipping of an exon of interest. The ASO-mediated exon skipping may be an approach to manipulate expression of a gene of interest. The expression of the gene of interest may be manipulated by ASOs to inhibit splicing of an exon, intron or a specific splice site of a gene of interest, leading to, e.g., without limitation, restoring the reading frame of a defective gene of interest, generating a different isoform of a gene of interest (e.g., a dominant negative isoform), skipping a toxic part of the gene, silencing the gene, and/or changing the structure and function of a gene. Examples of the exon of interest and gene of interest include, without limitation, those disclosed herein.

In some aspects, the present technology provides a use of the ASO (e.g., bipartite ASO) disclosed herein, the composition (e.g., pharmaceutical composition) comprising the ASO (e.g., bipartite ASO) as disclosed herein, and/or the vector encoding the ASO (e.g., bipartite ASO) as disclosed herein to treat a disease and/or complications thereof. Examples of the disease include, without limitation, those disclosed herein.

In some aspects, the present technology provides the ASO (e.g., bipartite ASO) disclosed herein, the composition (e.g., pharmaceutical composition) comprising the ASO (e.g., bipartite ASO) as disclosed herein, and/or the vector encoding the ASO (e.g., bipartite ASO) as disclosed herein for use in generating or promoting exon skipping of an exon of interest. The ASO-mediated exon skipping may be an approach to manipulate expression of a gene of interest. The expression of the gene of interest may be manipulated by ASOs to inhibit splicing of an exon, intron or a specific splice site of a gene of interest, leading to, e.g., without limitation, restoring the reading frame of a defective gene of interest, generating a different isoform of a gene of interest (e.g., a dominant negative isoform), skipping a toxic part of the gene, silencing the gene, and/or changing the structure and function of a gene. Examples of the exon of interest and gene of interest include, without limitation, those disclosed herein.

In some aspects, the present technology provides the ASO (e.g., bipartite ASO) disclosed herein, the composition (e.g., pharmaceutical composition) comprising the ASO (e.g., bipartite ASO) as disclosed herein, and/or the vector encoding the ASO (e.g., bipartite ASO) as disclosed herein for use in treating a disease and/or complications thereof. Examples of the disease and/or complications thereof include, without limitation, those disclosed herein.

In some aspects, the present technology provides a kit comprising the ASO (e.g., bipartite ASO) disclosed herein, the composition (e.g., pharmaceutical composition) comprising the ASO (e.g., bipartite ASO) as disclosed herein, and/or the vector encoding the ASO (e.g., bipartite ASO) as disclosed herein for use in generating or promoting exon skipping of an exon of interest. The ASO-mediated exon skipping may be an approach to manipulate expression of a gene of interest. The expression of the gene of interest may be manipulated by ASOs to inhibit splicing of an exon, intron or a specific splice site of a gene of interest, leading to, e.g., without limitation, restoring the reading frame of a defective gene of interest, generating a different isoform of a gene of interest (e.g., a dominant negative isoform), skipping a toxic part of the gene, silencing the gene, and/or changing the structure and function of a gene. Examples of the exon of interest and gene of interest include, without limitation, those disclosed herein.

In some aspects, the present technology provides a kit comprising the ASO (e.g., bipartite ASO) disclosed herein, the composition (e.g., pharmaceutical composition) comprising the ASO (e.g., bipartite ASO) as disclosed herein, and/or the vector encoding the ASO (e.g., bipartite ASO) as disclosed herein for use in treating a disease and/or complications thereof. Examples of the disease and/or complications thereof include, without limitation, those disclosed herein.

### BRIEF DESCRIPTION OF THE DRAWINGS

FIGS. 1A-1B show some schematic representations of ASOs of the present technology (e.g., bipartite ASOs) comprising 1) a “targeting” sequence that is fully or substantially complementary to a targeted region in a target nucleic acid (e.g., pre-mRNA), and 2) a “decoy” sequence that is located immediately adjacent to the targeting sequence (i.e., the decoy sequence is in a flanking region of the targeting sequence). FIG. 1A shows two schematic representations of bipartite ASOs where the decoy sequence is located immediately upstream at the 5′ end of the targeting sequence (left) and immediately downstream at the 3′ end of the targeting sequence (right), respectively. FIG. 1B shows one possible mechanism as to how a bipartite ASO of FIG. 1A generates or promotes skipping of exon of interest, where the decoy sequence located immediately upstream at the 5′ end of the targeting sequence, without being bound by any particular theory. The decoy sequence may resemble an optimal 5′ splice site and be complementary to part or all of the free single-strand sequence at the 5′ end of U1 snRNA, and thus the decoy sequence is able to interfere with the recognition of a nearby authentic 5′ splice site by U1 snRNA.

FIGS. 2A-2D illustrate impacts of the presence and/or the positions of decoy sequence 11 (SEQ ID No. 21) on exon 7 skipping in the SMN1 and SMN2 genes in HEK293 cells using ASOs comprising one of three targeting sequences (2203, 1938, and 0120). FIG. 2A shows a schematic diagram of the approximate binding locations of three tested targeting sequences (2203, 1938, and 0120) of the SMN1 and SMN2 genes. FIG. 2B shows the exon 7 skipping effects of ASOs comprising the targeting sequence 2203 in the SMN1 and SMN2 genes. The left panel of FIG. 2B shows a representative semi-quantitative fluorescent RT-PCR imaging analysis of SMN1 and SMN2 in HEK293 cells transfected with ASOs (12.5 nM, 25 nM, or 50 nM) having the targeting sequence 2203 alone (2203), the targeting sequence 2203 with decoy sequence 11 attached to the 5′ end (2203-L11), and the targeting sequence 2203 with decoy sequence 11 attached to the 3′ end (2203-R11), respectively. The middle panel of FIG. 2B shows the quantitation for exon 7 skipping (% excl) in SMN1 using the same ASOs presented as mean±standard deviation (n=3, **P<0.01 compared to 2203), with 2203 data shown on the left, 2203-L11 data on the middle, and 2203-R11 data on the right of the three columns for each concentration. The right panel of FIG. 2B shows the quantitation for exon 7 skipping (% excl) in SMN2 using the same ASOs presented as mean±standard deviation (n=3, **P<0.01 compared to 2203), with 2203 data shown on the left, 2203-L11 data on the middle, and 2203-R11 data on the right of the three columns for each concentration. Buffer was used as a negative control. FIG. 2C shows the exon 7 skipping effects of ASOs comprising the targeting sequence 1938 in SMN1 and SMN2. The left panel of FIG. 2C shows a representative semi-quantitative fluorescent RT-PCR imaging analysis of SMN1 and SMN2 in HEK293 cells transfected with ASOs (12.5 nM, 25 nM or 50 nM) having the targeting sequence 1938 alone (1938), the targeting sequence 1938 with decoy sequence 11 attached to the 5′ end (1938-L11), and the targeting sequence 1938 with decoy sequence 11 attached to the 3′ end (1938-R11), respectively. The middle panel of FIG. 2C shows the quantitation for exon 7 skipping (% excl) in SMN1 using the same ASOs presented as mean±standard deviation (n=3, *P<0.05, **P<0.01 compared to 1938), with 1938 data shown on the left, 1938-L11 data on the middle, and 1938-R11 data on the right of the three columns for each concentration. The right panel of FIG. 2C shows the quantitation for exon 7 skipping (% excl) in SMN2 using the same ASOs presented as mean±standard deviation (n=3, *P<0.05, **P<0.01 compared to 1938), with 1938 data shown on the left, 1938-L11 data on the middle, and 1938-R11 data on the right of the three columns for each concentration. Buffer was used as a negative control. FIG. 2D shows the exon 7 skipping effects of ASOs comprising the targeting sequence 0120 in SMN1 and SMN2. The left panel of FIG. 2D shows a representative semi-quantitative fluorescent RT-PCR imaging analysis of SMN1 and SMN2 in HEK293 cells transfected with ASOs (12.5 nM, 25 nM or 50 nM) having the targeting sequence 0120 alone (0120), the targeting sequence 0120 with decoy sequence 11 attached to the 5′end (0120-L11), and the targeting sequence 0120 with decoy sequence 11 attached to the 3′ end (0120-R11), respectively. The middle panel of FIG. 2D shows the quantitation for exon 7 skipping (% excl) in SMN1 using the same ASOs presented as mean±standard deviation (n=3, **P<0.01 compared to 0120), with 0120 data shown on the left, 0120-L11 on the middle, and 0120-R11 on the right of the three columns for each concentration. The right panel of FIG. 2D shows the quantitation for exon 7 skipping (% excl) in SMN2 using the same ASOs presented as mean±standard deviation (n=3, **P<0.01 compared to 0120), with 0120 data shown on the left, 0120-L11 data on the middle, and 0120-R11 data on the right of the three columns for each concentration. Buffer was used as a negative control.

FIGS. 3A-3B illustrate impacts of the presence and/or the positions of decoy sequence 11 on exon 51 skipping in the DMD gene in rhabdomyosarcoma (RD) cells using ASOs comprising one of four targeting sequences (148, 155, 165, and eteplirsen (Etep)). FIG. 3A shows the exon 51 skipping effects of ASOs comprising one of three targeting sequences (148, 155, and 165) in DMD gene. The left panel of FIG. 3A shows a representative semi-quantitative fluorescent RT-PCR imaging analysis, with RD cells transfected with ASOs (50 nM) having one of three targeting sequences with no decoy sequence (148, 155, and 165), decoy sequence 11 attached to the 5′ end of the targeting sequence (148-L11, 155-L11, and 165-L11), or decoy sequence 11 attached to the 3′end of the targeting sequence (148-R11, 155-R11, and 165-R11). The right panel of FIG. 3A shows the quantitation for exon 51 skipping (% excl) presented as mean±standard deviation (n=4, *P<0.05 (148-L11 vs 148), **P<0.01 (155-L11 vs 155, 165-L11 vs 165), in DMD in RD cells transfected with ASOs (50 nM) having one of three targeting sequences with no decoy sequence (148, 155, and 165), decoy sequence 11 attached to the 5′ end of the targeting sequence (148-L11, 155-L11, and 165-L11), or decoy sequence 11 attached to the 3′ end of the targeting sequence (148-R11, 155-R11, and 165-R11). Buffer was used as a negative control. FIG. 3B shows the exon 51 skipping effects of ASOs comprising the targeting sequence Etep in the DMD gene. The left panel of FIG. 3B shows a representative semi-quantitative fluorescent RT-PCR imaging analysis, with RD cells transfected with ASOs (12.5 nM, 25 nM or 50 nM) having the targeting sequence Etep with no decoy sequence (Etep), decoy sequence 11 attached to the 5′ end of the targeting sequence (Etep-L11), or decoy sequence 11 attached to the 3′ end of the targeting sequence (Etep-R11). The right panel of FIG. 3B shows the quantitation for exon 51 skipping (% excl) presented as mean±standard deviation (**P<0.01 (Etep-L11 vs Etep)), in DMD in RD cells transfected with ASOs (12.5 nM, 25 nM or 50 nM) having the targeting sequence with no decoy sequence (Etep, left of the three columns for each concentration), decoy sequence 11 attached to the 5′ end of the targeting sequence (Etep-L11, middle of the three columns for each concentration), or decoy sequence 11 attached to the 3′ end of the targeting sequence (Etep-R11, right of the three columns for each concentration). Buffer was used as a negative control.

FIG. 4 illustrates impacts of the presence, lengths, and/or the sequence of various decoy sequences (6a to 6f, 7a to 7c, 8a to 8d, 9a to 9c, 10a to 10b, 11, 12, and 13) attached to the 5′end of the targeting sequence Etep on exon 51 skipping in the DMD gene in RD cells. The upper panel of FIG. 4 shows a representative semi-quantitative fluorescent RT-PCR analysis of the DMD gene in RD cells transfected with ASOs (25 nM) having the targeting sequence Etep with no decoy sequence (Etep) or with decoy sequences of varying lengths attached to the 5′ end (Loa to L6f, L7a to L7c, L8a to L8d, L9a to L9c, L10a to L10b, L11, L12, and L13) of the targeting sequence Etep. Buffer was used as a negative control. The bottom panel of FIG. 4 shows the quantitation for exon 51 skipping (% excl) presented as mean±standard deviation (n=3, ^{#}P<0.05 (all vs Etep); *P<0.05, **P<0.01 (all vs Etep-L11)), in DMD in RD cells transfected with the same ASOs (25 nM) tested in the upper panel of FIG. 4 (Etep, Etep-L6a to Etep-L6f, Etep-L7a to Etep-L7e, Etep-L8a to Etep-L8d, Etep-L9a to Etep-L9c, Etep-L10a to Etep-L10b, Etep-L11, Etep-L12, and Etep-L13). Buffer was used as a negative control.

FIG. 5 illustrates impacts of the presence, lengths, and/or the sequence of various decoy sequences (7a to 7e, 8a to 8d, 9a to 9c, 10a to 10b, and 11) attached to the 5′ end of the targeting sequence 000A on exon 51 skipping in the DMD gene in RD cells. The upper panel of FIG. 5 shows a representative semi-quantitative fluorescent RT-PCR analysis of the DMD gene in RD cells transfected with ASOs (25 nM) having the targeting sequence 000A with no decoy sequence (000A) or with decoy sequences of varying lengths attached to the 5′ end (L7a to L7c, L8a to L8d, L9a to L9c, L10a to L10b, and L11) of the targeting sequence 000A. Buffer was used as a negative control. The bottom panel of FIG. 5 shows the quantitation for exon 51 skipping (% excl) presented as mean±standard deviation (n=3, *P<0.05, **P<0.01, all vs 000A), in DMD in RD cells transfected with the same ASOs (25 nM) tested in the upper panel of FIG. 5 (000A, 000A-L7a to 000A-L7c, 000A-L8a to 000A-L8d, 000A-L9a to 000A-L9c, 000A-L10a to 000A-L10b, and 000A-L11). Buffer was used as a negative control.

FIG. 6 illustrates impacts of the presence, lengths, and/or the sequence of various decoy sequences (7c, 8c, 9b, and 10a for the targeting sequence Etep, and 8c, and 9a to 9c for the targeting sequence 000A) attached to the 5′ end of the targeting sequence Etep or 000A on exon 51 skipping in the DMD gene in tibialis anterior and gastrocnemius of DMD-humanized mice. The left upper panel of FIG. 6 shows a representative semi-quantitative fluorescent RT-PCR analysis of DMD exon 51 skipping in tibialis anterior of DMD-humanized mice administered with ASOs having targeting sequence Etep) with no decoy sequence (Etep), or with decoy sequences of varying lengths attached to the 5′ end of the targeting sequence Etep (L7c, L8c, L9b, and L10a) or 000A (L8c, and L9a to L9c). Saline was used as a negative control. The right upper panel of FIG. 6 shows a representative semi-quantitative fluorescent RT-PCR analysis of DMD exon 51 skipping in gastrocnemius of DMD-humanized mice administered with ASOs having targeting sequence Etep with no decoy sequence (Etep), or with decoy sequences of varying lengths attached to the 5′ end of the targeting sequence Etep (L7c, L8c, L9b, and L10a) or 000A (L8c, and L9a to L9c). Saline was used as a negative control. The left bottom panel of FIG. 6 shows the quantitation for exon 51 skipping (% excl) presented as mean±standard deviation (n=3, *P<0.05, **P<0.01, ***P<0.001 (all vs Etep)), in tibialis anterior of DMD-humanized mice administered with the same ASOs tested in the left upper panel of FIG. 6 (Etep, Etep-L7c, Etep-L8c, Etep-L9b, Etep-L10a, 000A-L8c, and 000A-L9a to 000A-L9c). Saline was used as a negative control. The right bottom panel of FIG. 6 shows the quantitation for exon 51 skipping (% excl) presented as mean±standard deviation (n=3, *P<0.05, **P<0.01 (all vs Etep)), in gastrocnemius of DMD-humanized mice administered with the same ASOs tested in the right upper panel of FIG. 6 (Etep, Etep-L7c, Etep-L8c, Etep-L9b, Etep-L10a, 000A-L8c, and 000A-L9a to 000A-L9c). Saline was used as a negative control.

FIG. 7 illustrates impacts of the presence, positions, lengths, and/or the sequence of various decoy sequences (7a to 7e, 8a to 8d, 9a to 9c, 10a to 10b, and 11) attached to the 5′ end or the 3′ end of the targeting sequence viltolarsen (Vilto) on exon 53 skipping in the DMD gene in RD cells. The left upper panel of FIG. 7 shows a representative semi-quantitative fluorescent RT-PCR analysis of exon 53 skipping, in the DMD gene in RD cells transfected with ASOs (25 nM) having the targeting sequence Vilto alone (Vilto) or the targeting sequence Vilto with decoy sequences of varying lengths attached to the 5′ end (Vilto-L7a to Vilto-L7e, Vilto-L8a to Vilto-L8d, Vilto-L9a to Vilto-L9c, Vilto-L10a to Vilto-L10b, and Vilto-L11). Buffer was used as a negative control. The right upper panel of FIG. 7 shows a representative semi-quantitative fluorescent RT-PCR analysis of exon 53 skipping, in the DMD gene in RD cells transfected with ASOs (25 nM) having the targeting sequence Vilto alone (Vilto) or the targeting sequence Vilto with decoy sequences of varying lengths attached to the 3′ end (Vilto-R7a to Vilto-R7e, Vilto-R8a to Vilto-R8d, Vilto-R9a to Vilto-R9c, Vilto-R10a to Vilto-R10b, and Vilto-R11). Buffer was used as a negative control. The left bottom panel of FIG. 7 shows the quantitation for exon 53 skipping (% excl) presented as mean±standard deviation (n=3, *P<0.05, **P<0.01, ***P<0.001, all vs Vilto), in the DMD gene in RD cells transfected with the same ASOs (25 nM) tested in the left upper panel of FIG. 7 (Vilto, Vilto-L7a to Vilto-L7e, Vilto-L8a to Vilto-L8d, Vilto-L9a to Vilto-L9c, Vilto-L10a to Vilto-L10b, and Vilto-L11). Buffer was used as a negative control. The right bottom panel of FIG. 7 shows the quantitation for exon 53 skipping (% excl) presented as mean±standard deviation (n=3, *P<0.05, **P<0.01, all vs Vilto), in the DMD gene in RD cells transfected with the same ASOs (25 nM) tested in the right upper panel of FIG. 7 (Vilto, Vilto-R7a to Vilto-R7c, Vilto-R8a to Vilto-R8d, Vilto-R9a to Vilto-R9c, Vilto-R10a to Vilto-R10b, and Vilto-R11). Buffer was used as a negative control.

FIGS. 8A-8B illustrate impacts of the presence, positions, lengths, and/or the sequence of various decoy sequences (7a to 7e, 8a to 8d, 9a to 9c, 10a to 10b, and 11) attached to the 5′ end or the 3′ end of the targeting sequence 002A on skipping of part of exon 45 in the DMD gene in RD cells. FIG. 8A shows a schematic diagram of the activation of a cryptic 5′ splice site in DMD exon 45 by the tested targeting sequence 002A, which lead to loss of the last 32 nt of exon 45. The 32-nt loss, here referred to as partial skipping of exon 45, can restore reading frame the same as skipping of the whole 176-nt exon 45 for some DMD patients but with much less loss of protein sequence coded by exon 45. The left upper panel of FIG. 8B shows a representative semi-quantitative fluorescent RT-PCR analysis of partial exon 45 skipping, in the DMD gene in RD cells transfected with ASOs (25 nM) having the targeting sequence 002A alone (002A) or the targeting sequence 002A with decoy sequences of varying lengths attached to the 5′ end (002A-L7a to 002A-L7c, 002A-L8a to 002A-L8d, 002A-L9a to 002A-L9c, 002A-L10a to 002A-L10b, and 002A-L11). Buffer was used as a negative control. The right upper panel of FIG. 8B shows a representative semi-quantitative fluorescent RT-PCR analysis of partial exon 45 skipping, in the DMD gene in RD cells transfected with ASOs (25 nM) having the targeting sequence 002A alone (002A) or the targeting sequence 002A with decoy sequences of varying lengths attached to the 3′ end (002A-R7a to 002A-R7c, 002A-R8a to 002A-R8d, 002A-R9a to 002A-R9c, 002A-R10a to 002A-R10b, and 002A-R11). Buffer was used as a negative control. The left bottom panel of FIG. 8B shows the quantitation for partial exon 45 skipping (% excl) presented as mean±standard deviation (n=3, *P<0.05, **P<0.01 (all vs 002A)), in the DMD gene in RD cells transfected with the same ASOs (25 nM) tested in the left upper panel of FIG. 8B (002A, 002A-L7a to 002A-L7c, 002A-L8a to 002A-L8d, 002A-L9a to 002A-L9c, 002A-L10a to 002A-L10b, and 002A-L11). Buffer was used as a negative control. The right bottom panel of FIG. 8B shows the quantitation for partial exon 45 skipping (% excl) presented as mean±standard deviation (n=3, *P<0.05, all vs 002A), in DMD in RD cells transfected with the same ASOs (25 nM) tested in the right upper panel of FIG. 8B (002A, 002A-R7a to 002A-R7c, 002A-R8a to 002A-R8d, 002A-R9a to 002A-R9c, 002A-R10a to 002A-R10b, and 002A-R11). Buffer was used as a negative control.

FIGS. 9A-9B illustrate impacts of the presence, positions, lengths, and/or the sequence of various decoy sequences (7a to 7e, 8a to 8d, 9a to 9c, 10a to 10b, and 11) attached to the 5′ end or the 3′ end of the targeting sequence 014B on exon 17 skipping in the APP gene in HEK293 cells. The left panel of FIG. 9A shows a representative semi-quantitative fluorescent RT-PCR analysis of exon 17 skipping, in the APP gene in HEK293 cells transfected with ASOs (50 nM) having the targeting sequence 014B alone (014B) or the targeting sequence 014B with decoy sequences of varying lengths attached to the 5′ end (014B-L7a to 014B-L7c, 014B-L8a to 014B-L8d, 014B-L9a to 014B-L9c, 014B-L10a to 014B-L10b, and 014B-L11). Buffer was used as a negative control. The right panel of FIG. 9A shows the quantitation for exon 17 skipping (% excl) presented as mean±standard deviation (n=3, *P<0.05, **P<0.01, ***P<0.001 (all vs 014B)), in the APP gene in HEK293 cells transfected with the same ASOs (50 nM) tested in the left panel of FIG. 9A (014B, 014B-L7a to 014B-L7c, 014B-L8a to 014B-L8d, 014B-L9a to 014B-L9c, 014B-L10a to 014B-L10b, and 014B-L11). Buffer was used as a negative control. The left panel of FIG. 9B shows a representative of semi-quantitative fluorescent RT-PCR analysis of exon 17 skipping in the APP gene in HEK293 cells transfected with ASOs (50 nM) having the targeting sequence 014B alone (014B) or the targeting sequence 014B with decoy sequences of varying lengths attached to the 3′end (014B-R7a to 014B-R7c, 014B-R8a to 014B-R8d, 014B-R9a to 014B-R9c, 014B-R10a to 014B-R10b, and 014B-R11). Buffer was used as a negative control. The right panel of FIG. 9B shows the quantitation for exon 17 skipping (% excl) presented as mean±standard deviation (n=3, *P<0.05, **P<0.01, ***P<0.001 (all vs 014B)), in the APP gene in HEK293 cells transfected with the same ASOs (50 nM) tested in the left panel of FIG. 9B (014B, 014B-R7a to 014B-R7c, 014B-R8a to 014B-R8d, 014B-R9a to 014B-R9c, 014B-R10a to 014B-R10b, and 014B-R11). Buffer was used as a negative control.

FIGS. 10A-10B illustrate impacts of the presence, positions, lengths, and/or the sequence of various decoy sequences (7a to 7e, 8a to 8d, 9a to 9c, 10a to 10b, and 11) attached to the 5′ end or the 3′ end of the targeting sequence 017B on exon 41 skipping in the CEP290 gene in HEK293 cells. The left panel of FIG. 10A shows a representative of semi-quantitative fluorescent RT-PCR analysis of exon 41 skipping in the CEP290 gene in HEK293 cells transfected with ASOs (50 nM) having the targeting sequence 017B alone (017B) or the targeting sequence 017B with decoy sequences of varying lengths attached to the 5′ end (017B-L7a to 017B-L7c, 017B-L8a to 017B-L8d, 017B-L9a to 017B-L9c, 017B-L10a to 017B-L10b, and 017B-L11). Buffer was used as a negative control. The right panel of FIG. 10A shows the quantitation for exon 41 skipping (% excl) presented as mean±standard deviation (n=3, *P<0.05, **P<0.01, ***P<0.001 (all vs 017B)), in the CEP290 gene in HEK293 cells transfected with the same ASOs (50 nM) tested in the left panel of FIG. 10A (017B, 017B-L7a to 017B-L7c, 017B-L8a to 017B-L8d, 017B-L9a to 017B-L9c, 017B-L10a to 017B-L10b, and 017B-L11). Buffer was used as a negative control. The left panel of FIG. 10B shows a representative of semi-quantitative fluorescent RT-PCR analysis of exon 41 skipping in the CEP290 gene in HEK293 cells transfected with ASOs (50 nM) having the targeting sequence 017B alone (017B) or the targeting sequence 017B with decoy sequences of varying lengths attached to the 3′ end (017B-R7a to 017B-R7c, 017B-R8a to 017B-R8d, 017B-R9a to 017B-R9c, 017B-R10a to 017B-R10b, and 017B-R11). Buffer was used as a negative control. The right panel of FIG. 10B shows the quantitation for exon 41 skipping (% excl) presented as mean±standard deviation (n=3, **P<0.01, ***P<0.001 (all vs 017B)), in the CEP290 gene in HEK293 cells transfected with the same ASOs (50 nM) tested in the left panel of FIG. 10B (017B, 017B-R7a to 017B-R7c, 017B-R8a to 017B-R8d, 017B-R9a to 017B-R9c, 017B-R10a to 017B-R10b, and 017B-R11). Buffer was used as a negative control.

FIGS. 11A-11B illustrate impacts of the presence, positions, lengths, and/or the sequence of various decoy sequences (7a to 7e, 8a to 8d, 9a to 9c, 10a to 10b, and 11) attached to the 5′ end or the 3′ end of the targeting sequence 024B on exon 19 skipping in the HER2 gene (also called ERBB2) in Hela cells. The left panel of FIG. 11A shows a representative of semi-quantitative fluorescent RT-PCR analysis of exon 19 skipping in the HER2 gene in Hela cells transfected with ASOs (50 nM) having the targeting sequence 024B alone (024B) or the targeting sequence 024B with decoy sequences of varying lengths attached to the 5′ end (024B-L7a to 024B-L7c, 024B-L8a to 024B-L8d, 024B-L9a to 024B-L9c, 024B-L10a to 024B-L10b, and 024B-L11). Buffer was used as a negative control. The right panel of FIG. 11A shows the quantitation for exon 19 skipping (% excl) presented as mean±standard deviation (n=3, *P<0.05, **P<0.01, ***P<0.001 (all vs 024B)), in the HER2 gene in HeLa cells transfected with the same ASOs (50 nM) tested in the left panel of FIG. 11A (024B, 024B-L7a to 024B-L7c, 024B-L8a to 024B-L8d, 024B-L9a to 024B-L9c, 024B-L10a to 024B-L10b, and 024B-L11). Buffer was used as a negative control. The left panel of FIG. 11B shows a representative of semi-quantitative fluorescent RT-PCR analysis of exon 19 skipping in the HER2 gene in Hela cells transfected with ASOs (50 nM) having the targeting sequence 024B alone (024B) or the targeting sequence 024B with decoy sequences of varying lengths attached to the 3′ end (024B-R7a to 024B-R7c, 024B-R8a to 024B-R8d, 024B-R9a to 024B-R9c, 024B-R10a to 024B-R10b, and 024B-R11). Buffer was used as a negative control. The right panel of FIG. 11B shows the quantitation for exon 19 skipping (% excl) presented as mean±standard deviation (n=3, *P<0.05, **P<0.01 (all vs 024B)), in the HER2 gene in HeLa cells transfected with the same ASOs (50 nM) tested in the left panel of FIG. 11B (024B, 024B-R7a to 024B-R7c, 024B-R8a to 024B-R8d, 024B-R9a to 024B-R9c, 024B-R10a to 024B-R10b, and 024B-R11). Buffer was used as a negative control.

FIGS. 12A-12B illustrate impacts of the presence, positions, lengths, and/or the sequence of various decoy sequences (7a to 7e, 8a to 8d, 9a to 9c, 10a to 10b, and 11) attached to the 5′ end or the 3′ end of the targeting sequence 015C on exon 10 skipping in the ATXN3 gene (also called SCA3) in A549 cells. The left panel of FIG. 12A shows a representative semi-quantitative fluorescent RT-PCR analysis of exon 10 skipping in the ATXN3 gene in A549 cells transfected with ASOs (40 nM) having the targeting sequence 015C alone (015C) or the targeting sequence 015C with decoy sequences of varying lengths attached to the 5′ end (015C-L7a to 015C-L7c, 015C-L8a to 015C-L8d, 015C-L9a to 015C-L9c, 015C-L10a to 015C-L10b, and 015C-L11). Buffer was used as a negative control. The right panel of FIG. 12A shows the quantitation for exon 10 skipping (% excl) presented as mean±standard deviation (n=3, *P<0.05, **P<0.01, ***P<0.001 (all vs 015C)), in the ATXN3 gene in A549 cells transfected with the same ASOs (40 nM) tested in the left panel of FIG. 12A (015C, 015C-L7a to 015C-L7c, 015C-L8a to 015C-L8d, 015C-L9a to 015C-L9c, 015C-L10a to 015C-L10b, and 015C-L11). Buffer was used as a negative control. The left panel of FIG. 12B shows a representative semi-quantitative fluorescent RT-PCR analysis of exon 10 skipping in the ATXN3 gene in A549 cells transfected with ASOs (40 nM) having the targeting sequence 015C alone (015C) or the targeting sequence015C with decoy sequences of varying lengths attached to the 3′ end (015C-R7a to 015C-R7c, 015C-R8a to 015C-R8d, 015C-R9a to 015C-R9c, 015C-R10a to 015C-R10b, and 015C-R11). Buffer was used as a negative control. The right panel of FIG. 12B shows the quantitation for exon 10 skipping (% excl) presented as mean±standard deviation (n=3, *P<0.05, **P<0.01, ***P<0.001 (all vs 015C)), in the ATXN3 gene in A549 cells transfected with the same ASOs (40 nM) tested in left panel of FIG. 12B (015C, 015C-R7a to 015C-R7c, 015C-R8a to 015C-R8d, 015C-R9a to 015C-R9c, 015C-R10a to 015C-R10b, and 015C-R11). Buffer was used as a negative control.

FIGS. 13A-13B illustrate impacts of the presence, positions, lengths, and/or the sequence of various decoy sequences (7a to 7c, 8a to 8d, 9a to 9c, 10a to 10b, and 11) attached to the 5′ end or the 3′ end of the targeting sequence 027B on exon 10 skipping in the PKM gene in RD cells. The left panel of FIG. 13A shows a representative semi-quantitative fluorescent RT-PCR analysis of exon 10 skipping in the PKM gene in RD cells transfected with ASOs (50 nM) having the targeting sequence 027B alone (027B) or the targeting sequence 027B with decoy sequences of varying lengths attached to the 5′ end (027B-L7a to 027B-L7c, 027B-L8a to 027B-L8d, 027B-L9a to 027B-L9c, 027B-L10a to 027B-L10b, and 027B-L11). Buffer was used as a negative control. The right panel of FIG. 13A shows the quantitation for exon 10 skipping (% excl) presented as mean±standard deviation (n=3, **P<0.01, ***P<0.001 (all vs 027B)), in the PKM gene in RD cells transfected with the same ASOs (50 nM) tested in the left panel of FIG. 13A (027B, 027B-L7a to 027B-L7c, 027B-L8a to 027B-L8d, 027B-L9a to 027B-L9c, 027B-L10a to 027B-L10b, and 027B-L11). Buffer was used as a negative control. The left panel of FIG. 13B shows a representative semi-quantitative fluorescent RT-PCR analysis of exon 10 skipping in the PKM gene in RD cells transfected with ASOs (50 nM) having the targeting sequence 027B alone (027B) or the targeting sequence 027B with decoy sequences of varying lengths attached to the 3′ end (027B-R7a to 027B-R7c, 027B-R8a to 027B-R8d, 027B-R9a to 027B-R9c, 027B-R10a to 027B-R10b, and 027B-R11). Buffer was used as a negative control. The right panel of FIG. 13B shows the quantitation for exon 10 skipping (% excl) presented as mean±standard deviation (*P<0.05, **P<0.01, ***P<0.001 (all vs 027B)), in the PKM gene in RD cells transfected with the same ASOs (50 nM) tested in the left panel of FIG. 13B (027B, 027B-R7a to 027B-R7c, 027B-R8a to 027B-R8d, 027B-R9a to 027B-R9c, 027B-R10a to 027B-R10b, and 027B-R11). Buffer was used as a negative control.

FIGS. 14A-14B illustrate impacts of the presence, positions, lengths, and/or the sequence of various decoy sequences (7a to 7e, 8a to 8d, 9a to 9c, 10a to 10b, and 11) attached to the 5′ end or the 3′ end of the targeting sequence 029B on exon 6 skipping in the MDM4 gene in HEK293 cells. The left panel of FIG. 14A shows a representative semi-quantitative fluorescent RT-PCR analysis of exon 6 skipping in the MDM4 gene in HEK293 cells transfected with ASOs (50 nM) having the targeting sequence 029B alone (029B) or the targeting sequence 029B with decoy sequences of varying lengths attached to the 5′ end (029B-L7a to 029B-L7c, 029B-L8a to 029B-L8d, 029B-L9a to 029B-L9c, 029B-L10a to 029B-L10b, and 029B-L11). Buffer was used as a negative control. The right panel of FIG. 14A shows the quantitation for exon 6 skipping (% excl) presented as mean±standard deviation (n=3, *P<0.05, **P<0.01, ***P<0.001 (all vs 029B)), in the MDM4 gene in HEK293 cells transfected with the same ASOs (50 nM) tested in the left panel of FIG. 14A (029B, 029B-L7a to 029B-L7c, 029B-L8a to 029B-L8d, 029B-L9a to 029B-L9c, 029B-L10a to 029B-L10b, and 029B-L11). Buffer was used as a negative control. The left panel of FIG. 14B shows a representative semi-quantitative fluorescent RT-PCR analysis of exon 6 skipping in the MDM4 gene in HEK293 cells transfected with ASOs (50 nM) having the targeting sequence 029B alone (029B) or the targeting sequence 029B with decoy sequences of varying lengths attached to the 3′ end (029B-R7a to 029B-R7c, 029B-R8a to 029B-R8d, 029B-R9a to 029B-R9c, 029B-R10a to 029B-R10b, and 029B-R11). Buffer was used as a negative control. The right panel of FIG. 14B shows the quantitation for exon 6 skipping (% excl) presented as mean±standard deviation (n=3, *P<0.05, **P<0.01 (all vs 029B)), in the MDM4 gene in HEK293 cells transfected with the same ASOs (50 nM) tested in the left panel of FIG. 14B (029B, 029B-R7a to 029B-R7c, 029B-R8a to 029B-R8d, 029B-R9a to 029B-R9c, 029B-R10a to 029B-R10b, and 029B-R11). Buffer was used as a negative control.

### DETAILED DESCRIPTION

Exon skipping is a potential treatment approach for subjects having a disease and/or complications thereof that may benefit from the manipulation of expression of a gene of interest by ASOs to induce the exon skipping. ASOs may be used to generate or promote exon skipping, but it may be challenging to screen ASOs that provide desired efficiencies and efficacies for exon skipping. The expression of the gene of interest may be manipulated by ASOs to inhibit splicing of an exon, intron or a specific splice site of a gene of interest, leading to, e.g., without limitation, restoring the reading frame of a defective gene of interest, generating a different isoform of a gene of interest (e.g., a dominant negative isoform), skipping a toxic part of the gene, silencing the gene, and/or changing the structure and function of a gene. The manipulation of expression of the gene of interest may be beneficial in treating the diseases and/or complications thereof, although the gene of interest may or may not be a cause of the diseases. The diseases may be genetic or non-genetic disease (e.g., some non-genetic cancer, metabolic diseases or infectious diseases) as disclosed herein. The genetic diseases may or may not be associated with a splicing defect.

Most human genes contain introns and introns in nascent transcripts must be spliced out in the nucleus to generate a mature mRNA, followed by export into the cytoplasm for protein translation by ribosomes. However, genes often alternatively spliced, which not only contributes to expansion of transcript diversity but also serves as a mechanism to regulate gene function. One of the common alternative splicing modes is exon skipping. While exon skipping is a natural phenomenon frequently observed in the process of gene expression, it can be used as an approach to manipulate gene expression for therapeutic purposes. Depending on which exon is skipped, the outcome can be dramatically different. When a skipped exon is symmetric, i.e., the length of the exon can be divided by 3, the internally shortened protein isoform is to lose only amino acids coded by the exon. In the case that the skipped exon contains an essential part such as a protein localization signal, a motif associated with protein stability, or an enzymatic activity domain, the shortened protein isoform may change its localization or stability, lose function, or become a protein with dominant negative effects, or with distinct functions. In the case that the skipped exon is a poison exon, either natural or generated by mutations, exon skipping is to restore the function of the gene. When a skipped exon is asymmetric, i.e., the length of the exon cannot be divided by 3, the reading frame of the downstream exons is to be disrupted, which creates a premature termination codon (PTC) most likely in the next exon, often triggering nonsense-mediated mRNA decay (NMD). For the mRNA species with reading frame disruption but escaped from NMD, a C-terminal truncated protein isoform is to be generated, usually leading to its loss of function. In a word, exon skipping may generate an mRNA isoform or protein with similar, opposite, or distinct functions, or loss of function. Aartsma-Rus A, van Ommen G J. Antisense-mediated exon skipping: a versatile tool with therapeutic and research applications. RNA 2007 October; 13 (10): 1609-24. Doi: 10.1261/rna. 653607.

The exon skipping approach has wide applications. First, it can be used to skip a toxic part of an abnormal gene. One example of toxic exons is exon 10 of the ATXN3 gene in SCA3 patients who carry an abnormal expansion of CAG repeats in the exon. Second, exon skipping can be used to restore the reading frame disrupted by frameshift mutations observed in multiple genetic diseases. One of such diseases is called Duchenne muscular dystrophy. Moreover, exon skipping can be used to disrupt expression of deleterious gene products such as an oncoprotein, a virus protein, or a neurodegeneration-associated peptide. Finally, exon skipping can be used to generate a beneficial protein isoform. For example, skipping of PDCD1 exon 3 generates a PD1 isoform that has potential to treat cancer by acting as an PDL1/2 antibody. Toonen L J A, et al. Antisense Oligonucleotide-Mediated Removal of the Polyglutamine Repeat in Spinocerebellar Ataxia Type 3 Mice. Mol Ther Nucleic Acids. 2017 September; 8:232-242. doi: 10.1016/j. omtn. 2017.06.019. Lucía Echevarría L, et al. Exon-skipping advances for Duchenne muscular dystrophy. Human Molecular Genetics. 2018 August; 27 (R2): R163-R172. doi. org/10.1093/hmg/ddy 171. Chang J L, et al. Targeting Amyloid-β Precursor Protein, APP, Splicing with Antisense Oligonucleotides Reduces Toxic Amyloid-Production. Mol Ther. 2018 June; 26 (6): 1539-1551. doi: 10.1016/j. ymthe. 2018.02.029. Sun J, et al. Modulation of PDCD1 exon 3 splicing. RNA Biol. 2019 December; 16 (12): 1794-1805. Doi: 10.1080/15476286.2019.1659080.

As disclosed herein, without being bound by any particular theory, it was found that an ASO comprising or consisting of a decoy sequence (e.g., without limitation, the nucleotide sequences of SEQ ID Nos. 1 to 23 and 347 to 353) attached to a targeting sequence may be more effective and/or efficient in generating or promoting exon skipping than an ASO comprising the targeting sequence but not any decoy sequence as disclosed herein. Without being bound by any particular theory, the ASOs comprising the decoy sequences described herein surprisingly and unexpectedly improved efficiency of exon skipping by a targeting sequence. In some embodiments, when the decoy sequence is combined with a targeting sequence that otherwise has relatively low efficiency of promoting exon skipping, the resulting ASO becomes highly efficient in modulating exon skipping. Alternatively, an ASO comprising or consisting of a decoy sequence (e.g., the nucleotide sequences of SEQ ID Nos. 1 to 23 and 347 to 353) attached to a targeting sequence may achieve a comparable efficacy and/or efficiency in generating or promoting exon skipping at a lower dose than an ASO comprising the targeting sequence but not any decoy sequence as disclosed herein.

In certain embodiments, exon skipping effects and/or efficiencies may be quantified by the percentage of exclusion of the exon of interest (% excl) in total transcripts of each gene. In certain embodiments, the % excl of an ASO comprising a decoy sequence attached to a targeting sequence may be about 2.18 fold, about 2.3 fold, about 4 fold, over about 10 fold, over about 14 fold, over about 15 fold, over about 16 fold, or over about 17 fold of that of the ASO consisting of the targeting sequence. In certain embodiments, an ASO comprising a decoy sequence attached to a targeting sequence may achieve a comparable (e.g., at least about 70%, at least about 80%, at least about 90%, at least about 100%, at least about 110%, at least about 120%, at least about 130%, at least about 140%, at least about 150%) % excl of an ASO consisting of the targeting sequence at a dose of no more than about 25%, or no more than about 50% of the dose of the ASO consisting of the targeting sequence.

In certain embodiments, the optimal ASOs comprise a decoy sequence (e.g., without limitation, the nucleotide sequences of SEQ ID Nos. 1 to 23 and 347 to 353) attached to the 5′ end of the targeting sequence. In certain embodiments, the optimal ASOs comprise a decoy sequence (e.g., without limitation, the nucleotide sequences of SEQ ID Nos. 1 to 23 and 347 to 353) attached to the 3′ end of the targeting sequence. In certain embodiments, the ASO comprising a decoy sequence (e.g., without limitation, the nucleotide sequences of SEQ ID Nos. 1-23 and 347 to 353) attached to both the 3′ and 5′ ends of the targeting sequence. In certain embodiments the decoy sequence and the targeting sequence are attached without a linker.

Accordingly, provided herein in certain embodiments are methods of generating or promoting exon skipping of an exon of interest during pre-mRNA splicing comprising contacting a pre-mRNA in a cell or a subject with the ASO (e.g., bipartite ASO) disclosed herein, the composition (e.g., pharmaceutical composition) comprising the ASO (e.g., bipartite ASO) as disclosed herein, and/or the vector encoding the ASO (e.g., bipartite ASO) as disclosed herein. The ASO-mediated exon skipping may be an approach to manipulate expression of a gene of interest. The expression of the gene of interest may be manipulated by ASOs to inhibit splicing of an exon, intron or a specific splice site of a gene of interest, leading to, e.g., without limitation, restoring the reading frame of a defective gene of interest, generating a different isoform of a gene of interest (e.g., a dominant negative isoform), skipping a toxic part of the gene, silencing the gene, and/or changing the structure and function of a gene. In some embodiments, the method further comprises delivering to the cell or administering to the subject the ASO (e.g., bipartite ASO) disclosed herein.

Also provided herein in certain embodiments is a method of improving exon skipping efficacy and/or efficiency of a targeting sequence comprising obtaining one or more ASOs (e.g., bipartite ASOs) comprising the targeting sequence and a decoy sequence (e.g., without limitation, the nucleotide sequences of SEQ ID Nos. 1 to 23 and 347 to 353) operably connected at the 5′ end and/or 3′ end of the targeting sequence. In certain embodiments, the method further comprises screening and/or optimizing the one or more ASOs (e.g., bipartite ASOs) according to their exon skipping efficacies and/or efficiencies of an exon of interest. The exon skipping effects and/or efficiencies may be quantified by the percentage of exclusion of the exon of interest (% excl) in total transcripts of each gene. The targeting sequence may be capable of hybridizing to a sequence selected from the group consisting of an exon of interest, a flanking intron sequence upstream of the exon of interest, a flanking intron sequence downstream of the exon of interest, an intron-exon junction upstream of the exon of interest, and an intron-exon junction downstream of the exon of interest in a cell or subject.

Also provided herein in certain embodiments are methods of treating a disease and/or complications thereof in a subject comprising administering to the subject the ASO (e.g., bipartite ASO) disclosed herein, the pharmaceutical composition comprising the ASO (e.g., bipartite ASO) as disclosed herein, and/or the vector encoding the ASO (e.g., bipartite ASO) as disclosed herein.

Also provided herein are novel ASOs, each comprising a decoy sequence attached to a targeting sequence. In certain embodiments, the decoy sequence is selected from the group consisting of the nucleotide sequences of SEQ ID Nos. 1 to 23 and 347 to 353. In certain embodiments, the ASOs are capable of mediating skipping of one or more exons of a gene, such as the DMD gene (NCBI Gene ID: 1756), the SMN1 gene (NCBI Gene ID: 6606), the SMN2 gene (NCBI Gene ID: 6607), the APP gene (NCBI Gene ID: 351), the CEP290 gene (NCBI Gene ID: 80184), the HER2 gene (NCBI Gene ID: 2064), the ATXN3 gene (NCBI Gene ID: 4287), the PKM gene (NCBI Gene ID: 5315), and the MDM4 gene (NCBI Gene ID: 4194). This process is referred to as exon skipping. In certain embodiments, targeting sequences may target an exon, intron or a junction. In certain embodiments, the exon of interest is selected from the group consisting of exon 7 of the endogenous SMN1 and SMN2 genes, exon 51 of the endogenous DMD gene, exon 53 of the endogenous DMD gene, exon 45 of the endogenous DMD gene, exon 17 of the endogenous APP gene, exon 41 of the endogenous CEP290 gene, exon 19 of the endogenous HER2 gene, exon 10 of the ATXN3 gene, exon 10 of the endogenous PKM gene, and exon 6 of the endogenous MDM4 gene. In certain embodiments, the targeting sequence comprises or consists of a nucleotide sequence selected from the group consisting of the nucleotide sequences of SEQ ID Nos. 25, 29, 33, 37, 41, 45, 49, 75, 92, 124, 156, 188, 220, 252, 284, and 316. In certain embodiments, the ASO comprises or consists of a nucleotide sequence selected from the group consisting of nucleotide sequences of SEQ ID Nos. 26 to 27, 30 to 31, 34 to 35, 38 to 39, 42 to 43, 46 to 47, 50 to 73, 76 to 90, 93 to 122, 125 to 154, 157 to 186, 189 to 218, 221 to 250, 253 to 282, 285 to 314, 317 to 346, and 354 to 356. In certain embodiments, the targeting sequence targets a targeted sequence selected from the group consisting of the nucleotide sequences of SEQ ID Nos. 24, 28, 32, 36, 40, 44, 48, 74, 91, 123, 155, 187, 219, 251, 283, and 315.

In some embodiments, the ASO or a composition (e.g., pharmaceutical composition) comprising the ASO is useful for treatment of diseases and/or complications thereof treatable by skipping of exon 45, 51 or 53 of DMD gene. The ASO comprises or consists of a nucleotide sequence selected from the group consisting of nucleotide sequences of SEQ ID Nos. 38 to 39, 42 to 43, 46 to 47, 50 to 73, 76 to 90, 93 to 122, and 125 to 154. In certain embodiments, the disease is Duchenne's muscular dystrophy (DMD). DMD is caused by frameshift mutations of the DMD gene, and exon skipping strategies using an ASO or composition comprising an ASO disclosed herein can be employed to restore the reading frame and therefore treat DMD.

In some embodiments, the ASO or a composition (e.g., pharmaceutical composition) comprising the ASO is useful for treatment of diseases and/or complications thereof treatable by skipping of exon 7 of SMN1/2 gene. The ASO comprises or consists of a nucleotide sequence selected from the group consisting of nucleotide sequences of SEQ ID Nos. 26 to 27, 30 to 31, 34 to 35, and 354 to 356.

In some embodiments, the ASO or a composition (e.g., pharmaceutical composition) comprising the ASO is useful for treatment of diseases and/or complications thereof treatable by skipping of exon 17 of APP gene. The ASO comprises or consists of a nucleotide sequence selected from the group consisting of nucleotide sequences of SEQ ID Nos. 157 to 186.

In some embodiments, the ASO or a composition (e.g., pharmaceutical composition) comprising the ASO is useful for treatment of diseases and/or complications thereof treatable by skipping of exon 41 of CEP290 gene. The ASO comprises or consists of a nucleotide sequence selected from the group consisting of nucleotide sequences of SEQ ID Nos. 189 to 218.

In some embodiments, the ASO or a composition (e.g., pharmaceutical composition) comprising the ASO is useful for treatment of diseases and/or complications thereof treatable by skipping of exon 19 of HER2 gene. The ASO comprises or consists of a nucleotide sequence selected from the group consisting of nucleotide sequences of SEQ ID Nos. 221 to 250.

In some embodiments, the ASO or a composition (e.g., pharmaceutical composition) comprising the ASO is useful for treatment of diseases and/or complications thereof treatable by skipping of exon 10 of ATXN3 gene. The ASO comprises or consists of a nucleotide sequence selected from the group consisting of nucleotide sequences of SEQ ID Nos. 253 to 282.

In some embodiments, the ASO or a composition (e.g., pharmaceutical composition) comprising the ASO is useful for treatment of diseases and/or complications thereof treatable by skipping of exon 10 of PKM gene. The ASO comprises or consists of a nucleotide sequence selected from the group consisting of nucleotide sequences of SEQ ID Nos. 285 to 314.

In some embodiments, the ASO or a composition (e.g., pharmaceutical composition) comprising the ASO is useful for treatment of diseases and/or complications thereof treatable by skipping of exon 6 of MDM4 gene. The ASO comprises or consists of a nucleotide sequence selected from the group consisting of nucleotide sequences of SEQ ID Nos. 317 to 346.

The following description is merely exemplary in nature and is not intended to limit the present technology, its applications, or its uses. It should be understood that throughout the drawings, corresponding reference numerals indicate like or corresponding parts and features. The description of specific examples indicated in various embodiments of the present technology are intended for purposes of illustration only and are not intended to limit the scope of the present technology disclosed herein. Moreover, recitation of multiple embodiments having stated features is not intended to exclude other embodiments having additional features or other embodiments incorporating different combinations of the stated features.

Furthermore, the detailed description of various embodiments herein makes reference to the accompanying drawing/FIGS., which show various embodiments by way of illustration. While the embodiments are described in sufficient detail to enable those skilled in the art to practice the present technology, it should be understood that other embodiments may be realized and that logical and mechanical changes may be made without departing from the spirit and scope of the present technology. Thus, the detailed description herein is presented for purposes of illustration only and not of limitation. For example, steps or functions recited in descriptions, any method, system, or process, may be executed in any order and are not limited to the order presented. Moreover, any of the step or functions thereof may be outsourced to or performed by one or more third parties.

Furthermore, any reference to singular includes plural embodiments, and any reference to more than one component may include a singular embodiment.

### Definitions

Unless defined otherwise, all technical and scientific terms used herein have the same meaning as commonly understood by those of ordinary skill in the art to which the present technology belongs. For the purposes of the present technology, the following terms are defined below.

The articles “a” and “an” are used herein to refer to one or to more than one (i.e., to at least one) of the grammatical object of the article. By way of example, “an element” means one element or more than one element.

The term “about” means a quantity, level, value, number, frequency, percentage, dimension, size, amount, weight or length that varies by acceptable levels in the art. In some embodiments, such variation may be as much as 30, 25, 20, 15, 10, 9, 8, 7, 6, 5, 4, 3, 2 or 1% to a reference quantity, level, value, number, frequency, percentage, dimension, size, amount, weight or length. When the term “about” is used in conjunction with a numerical range, it modifies that range by extending the boundaries above and below the numerical values set forth.

The terms “administering” or “administer” include delivery of the therapeutic agent including ASOs of the present technology to a subject either by local or systemic administration. Administration may be topical (including ophthalmic and to mucous membranes including vaginal and rectal delivery), pulmonary (e.g., by inhalation or insufflation of powders or aerosols, including by nebulizer), intratracheal, intranasal, epidermal and transdermal, oral or parenteral. Parenteral administration includes intravenous, intraarterial, subcutaneous, intraperitoneal or intramuscular injection or infusion; or intracranial, e.g., intrathecal or intraventricular, administration.

The term “isolated” refers to a material that is substantially or essentially free from components that normally accompany it in its native state. For example, an “isolated oligonucleotide,” or “isolated oligomer” as used herein, may refer to an oligomer that has been purified or removed from the sequences that flank it in a naturally-occurring state, e.g., a DNA fragment that is removed from the sequences that are adjacent to the fragment in the genome.

The term “isolating” as it relates to cells may refer to the purification of cells (e.g., fibroblasts, lymphoblasts) from a source subject (e.g., a subject with an oligonucleotide repeat disease). In the context of mRNA or protein, “isolating” may refer to the recovery of mRNA or protein from a source, e.g., cells.

The term “functional” in reference to a protein includes the corresponding wildtype protein, as well as truncated forms of the wildtype protein derived from an mRNA transcript containing sequences corresponding to a truncated form of the transcript by having one or more exons missing while having sufficient biological activities to reduce the adverse effects of a defective protein present in subjects with a disease and/or complications thereof. A functional protein may have about 10%, 20%, 30%, 40%, 50%, 60%, 70%, 80%, 90%, or 100% (including all integers in between) of the in vitro or in vivo biological activity of the corresponding wildtype protein, as measured according to routine techniques in the art. The truncated protein that has less than full biological activity of the wildtype protein is sometimes referred to as “semi-functional” protein. Animal models are also valuable resources for studying the pathogenesis of disease, and provide a means to test disease-related activity, as in certain examples, a less than 100% biological activity may be sufficient to treat the disease.

The term “functional” in reference to a dystrophin protein includes those proteins derived from an mRNA transcript containing sequences corresponding to all of exons 1 to 79 of a dystrophin gene, also referred to as a wildtype protein. Also included are truncated forms of dystrophin derived from an mRNA transcript containing sequences corresponding to a truncated form of the transcript, for example, a dystrophin mRNA transcript having less than all of exons 1 to 79 of a dystrophin gene, such as those forms that are produced by some of the ASOs of the present technology. In other words, a truncated form of a dystrophin mRNA may exclude one or more exons of a corresponding dystrophin gene. A truncated form of a dystrophin mRNA may express a truncated or shortened form of a dystrophin protein, also referred to as a microdystrophin protein. A functional dystrophin protein refers generally to a dystrophin protein having sufficient biological activity to reduce the progressive degradation of muscle tissue that is otherwise characteristic of Duchenne muscular dystrophy, typically as compared to the altered or “defective” form of dystrophin protein that is present in some subjects with DMD or related disorders. A functional dystrophin protein may have about 10%, 20%, 30%, 40%, 50%, 60%, 70%, 80%, 90%, or 100% (including all integers in between) of the in vitro or in vivo biological activity of wildtype dystrophin, as measured according to routine techniques in the art. Dystrophin protein that has less than full biological activity of wildtype dystrophin is sometimes referred to as “semi-functional” dystrophin protein. As one example, dystrophin-related activity in muscle cultures in vitro can be measured according to myotube size, myofibril organization (or disorganization), contractile activity, and spontaneous clustering of acetylcholine receptors (see, e.g., Susan C. Brown, et al. Dystrophic phenotype induced in vitro by antibody blockade of muscle α-dystroglycan-laminin interaction. 1999 January. Journal of Cell Science. 112:209-216. doi: 10.1242/jcs. 112.2.209). Animal models are also valuable resources for studying the pathogenesis of disease, and provide a means to test dystrophin-related activity. Two of the widely used animal models for DMD research are the mdx mouse and the golden retriever muscular dystrophy (GRMD) dog, both of which are dystrophin negative (scc, e.g., C. A. Collins and J. E. Morgan. Duchenne's muscular dystrophy: animal models used to investigate pathogenesis and develop therapeutic strategies. 2003 August. Int J Exp Pathol. 84:165-172. doi: 10.1046/j. 1365-2613.2003.00354.

x). These and other animal models can be used to measure the functional activity of various dystrophin proteins.

The terms “DMD gene” and “dystrophin gene” here are used interchangeably to here and refer to the gene that encodes dystrophin. Similarly, the terms “DMD protein” and “dystrophin protein” and “dystrophin” here are used interchangeably and refer to the translated protein product of DMD gene.

“Exon skipping” refers generally to the process by which an entire exon, or a portion thereof, is removed from a given pre-RNA, and is thereby excluded from being present in the mature RNA, such as the mature mRNA that is translated into a protein. Hence, the portion of the protein that is otherwise encoded by the skipped exon is not present in the expressed form of the protein, typically creating an altered, though still functional, form of the protein. In some embodiments, the exon being skipped is an aberrant exon from the human dystrophin gene, which may contain a mutation or other alteration in its sequence that otherwise causes aberrant splicing. The terms “pre-mRNA” and “precursor mRNA” are used interchangeably and refer to a non-processed or partly processed precursor mRNA that is synthesized from a DNA template in the cell nucleus by transcription.

Within the context of the present technology, inducing and/or promoting skipping of an exon (or inducing or promoting exon skipping) as indicated herein means that at least 1%, 2%, 5%, 10%, 20%, 30%, 40%, 50%, 60%, 70%, 80%, 90% or 100% of the mRNA in one or more cells patient will not contain said exon. The level of exon skipping can be assessed by PCR as described in the examples.

The terms “complementary” and “complementarity” refer generally to the interaction of two nucleotide sequences that hybridize to form one double stranded molecule. The terms “complementary” and “complementarity,” in some embodiments, refer to polynucleotides (i.e., a sequence of nucleotides) related by base-pairing rules. For example, the sequence “T-G-A-C(5′-3′),” is complementary to the sequence “G-T-C-A (5′-3′).” Complementarity may be “partial”, in which only some of the nucleic acids' bases are matched in relation to a reference or targeted sequence, such as a targeted region, according to base pairing rules. Or, there may be “complete” or “exact” or “total” or “full” complementarity between the nucleic acids, which has the same meaning as “fully complementary” or “completely complementary,” in which all of the indicated nucleic acids' bases are matched according to base pairing rules. The degree of complementarity between nucleic acid strands has significant effects on the efficiency and strength of hybridization between nucleic acid strands. While complete complementarity is often desired, some embodiments can include one or more mismatches with respect to the targeted region. Variations at any location within the oligomer are included.

The term “substantially complementary” refer to polynucleotides that have at least 70% partial complementarity; that is, at least 70%, 75%, 80%, 85%, 90%, 95%, 98%, or 99% of the nucleic acids' bases are matched according to base pairing rules. The mismatches may be one or more base substitutions (mutations), or one or more base deletion, or one or more base insertions.

The terms “antisense oligonucleotide”, “antisense oligomer”, “antisense compound”, and “ASO” are used interchangeably and refer to an oligonucleotide comprising a targeting oligonucleotide sequence that is capable of hybridizing in a complementary fashion to a targeted sequence in a nucleic acid (either RNA or DNA, typically an RNA), such as a targeted region. In some embodiments, the ASO comprises a decoy sequence and a targeting sequence. In some embodiments, the targeting sequence of the ASO is fully or substantially complementary to the targeted region. In some embodiments, the targeted region is a sequence of the pre-RNA that is fully or partially complementary to the targeting sequence. The targeted region can be located at an exon of interest, a flanking intron sequence upstream of the exon of interest, a flanking intron sequence downstream of the exon of interest, an intron-exon junction upstream of the exon of interest, and an intron-exon junction downstream of the exon of interest. In some embodiments, the decoy sequence simulates an optimal 5′ splice site, and thus it serves as a 5′-splice-site decoy to interfere with the recognition of an adjacent authentic 5′ splice site by U1 snRNA. In some embodiments, the ASO comprises one or more DNA nucleotides, one or more RNA nucleotides, and mixtures thereof. Thus, although the sequences disclosed herein are presented in DNA form, said sequences also include the corresponding RNA form. In certain embodiments, one or more of the one or more DNA nucleotides and/or RNA nucleotides may be modified nucleotides. In some embodiments, the ASO further comprises one or more additional chemical moieties. In some embodiments, the additional chemical moiety is covalently linked to the 5′ end and/or the 3′ end of the decoy-targeting sequence moiety. In some embodiments, the additional chemical moiety is a cell penetrating peptide.

The term “modified nucleotide” includes any chemical moiety which differs structurally from a natural nucleotide but is capable of performing at least one function of a natural nucleotide. In some embodiments, a modified nucleotide comprises a modification at a sugar, base and/or internucleotidic linkage. In some embodiments, a modified nucleotide comprises a modified sugar, modified nucleobase and/or modified internucleotidic linkage. In some embodiments, a modified nucleotide is capable of at least one function of a nucleotide, e.g., forming a subunit in a polymer capable of base-pairing to a nucleic acid comprising an at least complementary sequence of bases.

The term “moiety” refers to a specific segment or functional group of a molecule. Chemical moieties are often recognized chemical entities embedded in or appended to a molecule. In some embodiments, a moiety of a compound is a monovalent, bivalent, or polyvalent group formed from the compound by removing one or more-H and/or equivalents thereof from a compound. In some embodiments, depending on its context, “moiety” may also refer to a compound or entity from which the moiety is derived from.

An “exon” refers to a defined section of nucleic acid that encodes for a protein, or a nucleic acid sequence that is represented in the mature form of an RNA molecule after either portions of a pre-processed (or precursor) RNA have been removed by splicing. The mature RNA molecule can be a messenger RNA (mRNA) or a functional form of a non-coding RNA, such as rRNA or tRNA. The human dystrophin gene has about 79 exons.

An “intron” refers to a nucleic acid region (within a gene) that is not translated into a protein. An intron is a non-coding section that is transcribed into a precursor RNA (pre-RNA), and subsequently removed by splicing during formation of the mature RNA.

The term “in vitro” refers to events that occur in an artificial environment, e.g., in a test tube or reaction vessel, in cell culture, etc., rather than within an organism (e.g., animal, plant, and/or microbe).

The term “in vivo” refers to events that occur within an organism (e.g., animal, plant, or microbe).

The term “gene” is intended to mean a genomic gene and also include cDNA, mRNA precursor (i.e., pre-mRNA) and mRNA.

The term “pharmaceutically acceptable salt” refers to salts prepared from pharmaceutically acceptable non-toxic acids or bases including inorganic acids and bases and organic acids and bases. For example, for compounds that contain a basic nitrogen, salts may be prepared from pharmaceutically acceptable non-toxic acids including inorganic and organic acids. Suitable pharmaceutically acceptable acid addition salts for the compounds of the present technology include acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. When the compounds contain an acidic side chain, suitable pharmaceutically acceptable base addition salts for the compounds of the present technology include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine), and procaine.

The term “pharmaceutical composition” means a composition comprising a compound as described herein and at least one component comprising pharmaceutically acceptable carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms.

The term “pharmaceutically acceptable carrier” is used to mean any carrier, diluent, adjuvant, excipient, or vehicle, as described herein, that is non-toxic and safe for human use. Examples of suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monosterate and gelatin. Examples of suitable carriers, diluents, solvents, or vehicles include water, ethanol, polyols, suitable mixtures thereof, vegetable oils (such as olive oil), and injectable organic esters such as ethyl oleate. Examples of excipients include lactose, milk sugar, sodium citrate, calcium carbonate, and dicalcium phosphate. Examples of disintegrating agents include starch, alginic acids, and some complex silicates. Examples of lubricants include magnesium stearate, sodium lauryl sulphate, talc, as well as high molecular weight polyethylene glycols.

The term “pharmaceutically acceptable” means it is, within the scope of sound medical judgment, suitable for use in contact with the cells of humans and lower animals without undue toxicity, irritation, allergic response and the like, and are commensurate with a reasonable benefit/risk ratio.

The term “immediately” when used in reference to an upstream or downstream sequence refers to a direct covalent linkage between two groups, and no intervening nucleotides.

The term “flanking” is used to refer to nucleotide sequences which are directly attached to one another, having no intervening nucleotides. By way of example, the pentanucleotide 5′-AAAAA-3′ is flanking the trinucleotide 5′-TTT-3′ when the two are connected as in: 5′-AAAAATTT-3′ or 5′-TTTAAAAA-3′, but not when the two are connected as in: 5′-AAAAACTTT-3′. In the latter case, the C nucleotide is said to be “interposed” between the pentanucleotide and the trinucleotide.

The terms “therapeutically-effective amount,” “therapeutically effective amount,” and “effective amount” as used herein are interchangeable and refer to the amount of a therapy (e.g., an ASO or its pharmaceutical composition provided herein) which is sufficient to reduce and/or ameliorate the severity and/or duration of a given disease and/or a symptom related thereto. This term also encompasses an amount necessary for the reduction or amelioration of the advancement or progression of a given disease, reduction or amelioration of the recurrence, development or onset of a given disease, and/or to improve or enhance the prophylactic or therapeutic effect(s) of another therapy (e.g., a therapy other than an ASO provided herein).

The terms “decoy,” “decoy sequence,” “ASO decoy,” “ASO decoy sequence,” and “5′-splice-site decoy” are used interchangeably herein to refer to nucleotide sequences that are fully or partially complementary to the single-strand 5′ end of U1 snRNA. In some embodiments, the decoy sequence simulates an optimal 5′ splice site, and thus it serves as a 5′-splice-site decoy to interfere with the recognition of an adjacent authentic 5′ splice site by U1 snRNA.

### Overview

Disclosed herein, in various embodiments, are ASOs for treating diseases and/or complications thereof that can be treated by exon skipping in a gene of interest (e.g., without limitation, SMN1, SMN2, DMD, APP, CEP290, HER2, SCA3, PKM, and MDM4). In certain embodiments, the ASOs (e.g., bipartite ASOs) comprises two components: 1) a “targeting” sequence that is fully or substantially complementary to a targeted region in the gene of interest, and 2) a “decoy” sequence that is located immediately upstream from the 5′ end and/or immediately downstream from the 3′ end of the targeting sequence. In some embodiments, the decoy sequence is located immediately upstream from the 5′ end of the targeting sequence. In certain embodiments, the decoy sequence is located immediately downstream from the 3′ end of the targeting sequence. In some embodiments, the decoy sequence has low sequence complementarity, such as less than about 80%, to the corresponding flanking sequence immediately adjacent to the 5′ or 3′ end of the targeted region in the target nucleic acid (e.g., mRNA).

In some embodiments, the ASO (e.g., bipartite ASO) further comprises a linker between the decoy sequence and the targeting sequence.

In some embodiments, the ASOs (e.g., the bipartite ASOs) of the present technology can bind to a transcript (e.g., pre-mRNA) of a gene of interest (e.g., SMN1, SMN2, DMD, APP, CEP290, HER2, SCA3, PKM, and MDM4), and change the splicing pattern of the transcript by inducing skipping of one or more exons. In some embodiments, the ASOs (e.g., the bipartite ASOs) of the present technology cause skipping of one or more of exons in mRNA with the exon skipping percentage (% excl) increased by about 10% or higher, about 20% or higher, about 30% or higher, about 40% or higher, about 50% or higher, about 60% or higher, about 70% or higher, about 80% or higher, and about 90% or higher. The exon skipping percentage can be measured using techniques known in the art, and as described throughout the instant application, for example, in the “assay” section below.

The presence of a decoy sequence in a bipartite ASO may increase the percentage of exon skipping by the targeting sequence. In some embodiments, the presence of a decoy sequence in the bipartite ASO may increase exon skipping percentage of the targeting sequence by at least about 2 fold compared to an ASO having the identical targeting sequence but no decoy sequence. In some embodiments, the presence of the decoy sequence in the bipartite ASO increases the exon skipping percentage of the targeting sequence by about 2 fold or higher, about 2.18 fold or higher, about 2.3 fold or higher, about 3 fold or higher, about 4 fold or higher, about 5 fold or higher, about 6 fold or higher, about 7 fold or higher, about 8 fold or higher, about 9 fold or higher, about 10 fold or higher, about 11 fold or higher, about 12 fold or higher, about 13 fold or higher, about 14 fold or higher, about 15 fold or higher, about 16 fold or higher or higher, about 17 fold or higher, about 18 fold or higher, about 19 fold or higher, about 20 fold or higher, about 25 fold or higher, about 30 fold or higher, about 40 fold or higher, about 50 fold or higher, about 60 fold or higher, about 70 fold or higher, about 80 fold or higher, about 90 fold or higher, about 100 fold or higher, or more, including all values and ranges between these values, compared to an ASO having the identical targeting sequence but no decoy sequence. In certain embodiments, an ASO comprising or consisting of a decoy sequence (e.g., the nucleotide sequences of SEQ ID Nos. 1 to 23 and 347 to 353) attached to a targeting sequence may achieve a comparable efficacy and/or efficiency in generating or promoting exon skipping at a lower dose than an ASO comprising the targeting sequence but not any decoy sequence as disclosed herein.

Provided herein in certain embodiments are compositions comprising the ASO (e.g., bipartite ASO) disclosed herein and a pharmaceutically acceptable carrier. In certain embodiments, the composition is a pharmaceutical formulation or composition.

Provided herein in certain embodiments is a vector encoding the ASO (e.g., bipartite ASO) disclosed herein.

Provided herein in certain embodiments are methods of generating or promoting exon skipping of an exon of interest during pre-mRNA splicing comprising contacting a pre-mRNA in a cell or a subject with the ASO (e.g., bipartite ASO) disclosed herein, the composition (e.g., pharmaceutical composition) comprising the ASO (e.g., bipartite ASO) as disclosed herein, and/or the vector encoding the ASO (e.g., bipartite ASO) as disclosed herein. The ASO-mediated exon skipping may be an approach to manipulate expression of a gene of interest. The expression of the gene of interest may be manipulated by ASOs to inhibit splicing of an exon, intron or a specific splice site of a gene of interest, leading to, e.g., without limitation, restoring the reading frame of a defective gene of interest, generating a different isoform of a gene of interest (e.g., a dominant negative isoform), skipping a toxic part of the gene, silencing the gene, and/or changing the structure and function of a gene.

Provided herein in certain embodiments are methods for improving exon skipping efficacy and/or efficiency of a targeting sequence comprising obtaining one or more ASOs (e.g., bipartite ASOs) comprising the targeting sequence and a decoy sequence as disclosed herein operably connected at the 5′ end and/or 3′ end of the targeting sequence. In certain embodiments, multiple ASOs (e.g., bipartite ASOs) may be provided for screening and optimization to provide one or more ASOs (e.g., bipartite ASOs) having improved exon skipping efficiency, e.g., without limitation, by the method further comprises screening and/or optimizing the one or more ASOs (e.g., bipartite ASOs) according to their exon skipping efficacies and/or efficiencies of an exon of interest.

Also provided herein in certain embodiments are methods of treating a disease and/or complications thereof in a subject comprising administering to the subject the ASO (e.g., bipartite ASO) disclosed herein, the composition (e.g., pharmaceutical composition) comprising the ASO (e.g., bipartite ASO) as disclosed herein, and/or the vector encoding the ASO (e.g., bipartite ASO) as disclosed herein. The ASO (e.g., bipartite ASO) may generate or promote exon skipping of the exon of interest during pre-mRNA splicing. The ASO-mediated exon skipping may be an approach to manipulate expression of the gene of interest. The expression of the gene of interest may be manipulated by ASOs to inhibit splicing of an exon, intron or a specific splice site of a gene of interest, leading to, e.g., without limitation, restoring the reading frame of a defective gene of interest, generating a different isoform of a gene of interest (e.g., a dominant negative isoform), skipping a toxic part of the gene, silencing the gene, and/or changing the structure and function of a gene. The manipulation of expression of the gene of interest may be beneficial in treating the diseases and/or complications thereof, although the gene of interest may or may not be a cause of the diseases. The diseases may be genetic or non-genetic disease (e.g., some non-genetic cancer, metabolic diseases or infectious diseases) as disclosed herein. The genetic diseases may or may not be associated with mutations related to splicing defect.

Also provided herein in certain embodiments is a kit comprising the ASO (e.g., bipartite ASO) disclosed herein, the composition (e.g., pharmaceutical composition) comprising the ASO (e.g., bipartite ASO) as disclosed herein, and/or the vector encoding the ASO (e.g., bipartite ASO) as disclosed herein for use in generating or promoting exon skipping of an exon of interest. The ASO-mediated exon skipping may be an approach to manipulate expression of a gene of interest. The expression of the gene of interest may be manipulated by ASOs to inhibit splicing of an exon, intron or a specific splice site of a gene of interest, leading to, e.g., without limitation, restoring the reading frame of a defective gene of interest, generating a different isoform of a gene of interest (e.g., a dominant negative isoform), skipping a toxic part of the gene, silencing the gene, and/or changing the structure and function of a gene.

Examples of gene of interest and diseases that may be treatable by exon skipping

### Dystrophin and Duchenne Muscular Dystrophy

Duchenne Muscular Dystrophy (DMD) is an X-linked, progressive muscle wasting disease caused by mutations in the DMD gene that abolish the production of dystrophin protein. Dystrophin is a rod-shaped cytoplasmic protein, and a vital part of the protein complex that connects the cytoskeleton of a muscle fiber to the surrounding extracellular matrix through the cell membrane. Dystrophin contains multiple functional domains. For instance, dystrophin contains an actin binding domain at about amino acids 14-240 and a central rod domain at about amino acids 253-3040. This large central domain is formed by 24 spectrin-like triple-helical elements of about 109 amino acids, which have homology to alpha-actinin and spectrin. The repeats are typically interrupted by four proline-rich non-repeat segments, also referred to as hinge regions. Repeats 15 and 16 are separated by an 18 amino acid stretch that appears to provide a major site for proteolytic cleavage of dystrophin. The sequence identity between most repeats ranges from 10-25%. One repeat contains three alpha-helices: 1, 2 and 3. Alpha-helices 1 and 3 are each formed by 7 helix turns, probably interacting as a coiled-coil through a hydrophobic interface. Alpha-helix 2 has a more complex structure and is formed by segments of four and three helix turns, separated by a Glycine or Proline residue. Each repeat is encoded by two exons, typically interrupted by an intron between amino acids 47 and 48 in the first part of alpha-helix 2. The other intron is found at different positions in the repeat, usually scattered over helix-3. Dystrophin also contains a cysteine-rich domain at about amino acids 3080-3360, including a cysteine-rich segment (i.e., 15 Cysteines in 280 amino acids) showing homology to the C-terminal domain of the slime mold (Dictyostelium discoideum) alpha-actinin. The carboxy-terminal domain is at about amino acids 3361-3685. The amino-terminus of dystrophin binds to F-actin and the carboxy-terminus binds to the dystrophin-associated protein complex (DAPC) at the sarcolemma. The DAPC includes the dystroglycans, sarcoglycans, integrins and caveolin, and mutations in any of these components cause autosomally inherited muscular dystrophies. The DAPC is destabilized when dystrophin is absent, which results in diminished levels of the member proteins, and in turn leads to progressive fiber damage and membrane leakage. In various forms of muscular dystrophy, such as Duchenne's muscular dystrophy (DMD) and Becker's muscular dystrophy (BMD), muscle cells produce an altered and functionally defective form of dystrophin, or no dystrophin at all, mainly due to mutations in the gene sequence that lead to incorrect splicing. The predominant expression of the defective dystrophin protein, or the complete lack of dystrophin or a dystrophin-like protein, leads to rapid progression of muscle degeneration, as noted above. In this regard, a “defective” dystrophin protein may be characterized by the forms of dystrophin that are produced in some subjects with DMD or BMD, as known in the art, or by the absence of detectable dystrophin.

Over 7,000 different mutations have been reported for DMD patients. Most patients (˜65%) carry large deletions involving one or more exons, but large duplications (˜12%) and small mutations (20%) are frequently reported as well. The commonality of these mutations is that they all result in nonfunctional dystrophins. For example, a deletion of exon 45 is one of the most common deletions found in DMD patients, whereas a deletion of exons 44 and 45 is generally associated with BMD. Thus, if exon 44 could be bypassed in pre-messenger RNA (mRNA) transcripts of these DMD patients, this would restore the reading frame and enable the production of a partially functional BMD-like dystrophin. In fact, it appears that many patients with a deletion bordering on exon 44, skip exon 44 spontaneously, although at very low levels. This results in slightly increased levels of dystrophin when compared with DMD patients carrying other deletions, and most likely underlies the less severe disease progression observed in these patients compared with DMD patients with other deletions.

### Exon Skipping for DMD Treatment

Antisense-mediated exon skipping induces skipping of one or more target exons and can be used to restore defective reading frames. Mutations in the dystrophin gene are amenable to therapeutic exon skipping. For example, mutations in the following exons are amenable to exon 51 skipping, e.g.: 45 to 50, 47 to 50, 48 to 50, 49 to 50, 50, 52, 52 to 63 (Leiden Duchenne muscular dystrophy mutation database, Leiden University Medical Center, The Netherlands). Determining whether a patient has a mutation in the DMD gene that is amenable to exon skipping is possible (scc, e.g., Aartsma-Rus, et al. Theoretic applicability of antisense-mediated exon skipping for Duchenne muscular dystrophy mutations. 2009 Feb. 24. Hum Mut. 30:293-299. doi: 10.1002/humu. 20918; Stephen Abbs, et al. Best Practice Guidelines on molecular diagnostics in Duchenne/Becker muscular dystrophies. 2010 June. Neuromusc Disorders. 20:422-427. doi: 10.1016/j. nmd. 2010.04.005, the disclosures of each of which are incorporated herein by reference in their entirety). To develop exon-skipping approach for patients with specific defects in the DMD gene, ASOs with targeting sequences targeting additional exons of the gene would have to be developed. For example, ASOs can be developed to skip exon 51, which is defective in 13%-14% of DMD patients.

Relevant physiological or cellular responses to the treatments included in this technology (in vivo or in vitro) will be apparent to persons skilled in the art, and may include reductions in the symptoms or pathology of Duchenne muscular dystrophy (DMD) and related disorders, such as Becker muscular dystrophy (BMD), limb-girdle muscular dystrophy, congenital muscular dystrophy, facioscapulohumeral muscular dystrophy, myotonic muscular dystrophy, oculopharyngeal muscular dystrophy, distal muscular dystrophy, Emery-Dreifuss muscular dystrophy, muscle wasting conditions or disorders, such as AIDS, cancer or chemotherapy related muscle wasting, and fibrosis or fibrosis-related disorders (for example, skeletal muscle fibrosis). An “increase” in a response may be “statistically significant” as compared to the response produced by a subject in need thereof in the absence of administration of an ASO compound and/or therapeutic (e.g., when compared to the “native” or “natural” rate of expression of a specific subject or cohort) or when compared to a control compound.

In some embodiments, in a DMD patient, a DMD allele contains a mutation in an exon, and the disorder can be treated by skipping of one or more exons of DMD gene transcripts. In some embodiments, in a DMD patient, a DMD allele or DMD transcript has a mutation in an exon(s), which is a missense or nonsense mutation, deletion, insertion, inversion, or translocation or duplication. In some embodiments, in a treatment for muscular dystrophy, one or more exons within DMD transcript are skipped, wherein the exon(s) encode a string of amino acids not essential for DMD protein function, or whose skipping can provide a fully or at least partially functional DMD protein. DMD protein here refers to dystrophin. In some embodiments, in a treatment for muscular dystrophy, an ASO is capable of mediating skipping of DMD exon 51 or 53, thereby creating an mRNA from which can be translated into an artificially internally truncated DMD protein variant which provides at least partially improved or fully restored biological activity (e.g., a functional DMD protein variant). In some embodiments, an internally truncated DMD protein variant produced from a dystrophin DMD transcript with one or more skipped exons is more functional than a terminally truncated DMD protein e.g., produced from a dystrophin DMD transcript with an out-of-frame deletion. In some embodiments, an internally truncated DMD protein variant produced from a dystrophin DMD transcript with one or more skipped exons is more resistant to nonsense-mediated decay, which can degrade a terminally truncated DMD protein, e.g., produced from a dystrophin DMD transcript with an out-of-frame deletion. In some embodiments, restoring the reading frame can convert an out-of-frame mutation to an in-frame mutation; in some embodiments, in humans, such a change can transform severe Duchenne Muscular Dystrophy into milder Becker Muscular Dystrophy.

Methods of the present technology may alleviate one or more characteristics of a myogenic or muscle cell of a patient or alleviate one or more symptoms of a DMD patient having a deletion including but not limited to exons 44, 44-46, 44-47, 44-48, 44-49, 44-51, 44-53 (correctable by exon 43 skipping), 19-45, 21-45, 43-45, 45, 47-54, 47-56 (correctable by exon 46 skipping), 51, 51-53, 51-55, 51-57 (correctable by exon 50 skipping), 13-50, 19-50, 29-50, 43-50, 45-50, 47-50, 48-50, 49-50, 50, 52 (correctable by exon 51 skipping), exons 8-51, 51, 53, 53-55, 53-57, 53-59, 53-60, (correctable by exon 52 skipping) and exons 10-52, 42-52, 43-52, 45-52, 47-52, 48-52, 49-52, 50-52, 52 (correctable by exon 53 skipping) in the DMD gene, occurring in a total of 68% of all DMD patients with a deletion (Aartsma-Rus, et al. Theoretic applicability of antisense-mediated exon skipping for Duchenne muscular dystrophy mutations. 2009 Feb. 24. Hum Mut. 30:293-299. Doi: 10.1002/humu. 20918). Sec U.S. Pat. No. 9,499,818, incorporated by reference in its entirety herein.

### SMN Genes and Spinal Muscular Atrophy

The SMN1 and SMN2 genes are associated with a disease called spinal muscular atrophy (SMA). Both genes encode an identical protein (survival of motor neuron, or SMN). One difference between the two genes is that SMN2 differs by a C to T transition in exon 7 that causes substantial skipping of this exon, such that SMN2 expresses only low levels of functional protein.

Additional genes of interest and associated diseases that may be treatable by exon skipping

Examples of additional genes of interest include, without limitation, the APP gene, the CEP290 gene, the HER2 gene, the PKM gene, and the MDM4 gene. The APP gene is associated with Alzheimer's diseases and the CEP290 gene is associated with Joubert Syndrome. ASOs that promote exon 19 of the HER2 gene, exon 10 of the PKM gene, or exon 6 of the MDM4 gene have potential to treat various types of cancer comprising breast cancer, HER2-positive biliary tract, colorectal, non-small-cell lung, bladder cancers, prostate cancer, lung cancer, cervix cancer, kidney cancer, papillary thyroid cancer, colon cancer, colorectal cancer, gliomas, ovarian cancer, gastric cancer, hepatoblastoma, fibrolamellar, hepatocellular, carcinoma, soft tissue sarcoma, osteosarcoma, chronic lymphocytic, leukemia, acute myeloid leukemia, mantle cell lymphoma, Pediatric Burkitt, lymphoma, salivary gland cancer, liver cancer, and melanoma. (Sec, e.g., Do-Youn Oh and Yung-Juc Bang. HER2-targeted therapies-a role beyond breast cancer. 2019 Sep. 23. Nat. Rev. Clin. Oncol. 17:33-48. Doi: 10.1038/s41571-019-0268-3; K. Zahra, et al. Pyruvate kinase M2 and cancer: the role of PKM2 in promoting tumorigenesis. 2020 Mar. 2. Front. Oncol. 10:159. Doi: 10.3389/fonc. 2020.00159; and D. Yu, et al. Targeting MDMX for cancer therapy: rationale, strategies, and challenges. 2020 Aug. 5. Front. Oncol. 10:1389. Doi: 10.3389/fonc. 2020.01389.)

### Bipartite ASO for Pre-mRNA Splicing Modulation Via Exon Skipping

In some aspects, the present technology provides a method of generating or promoting exon skipping of an exon of interest during pre-mRNA splicing comprising contacting a pre-mRNA in a cell or a subject with the ASO (e.g., bipartite ASO) disclosed herein, the composition (e.g., pharmaceutical composition) comprising the ASO (e.g., bipartite ASO) as disclosed herein, and/or the vector encoding the ASO (e.g., bipartite ASO) as disclosed herein a vector encoding the ASO (e.g., bipartite ASO) disclosed herein. In some embodiments, the method further comprises delivering to the cell or administering to the subject the ASO (e.g., bipartite ASO) disclosed herein. The ASO-mediated exon skipping may be an approach to manipulate expression of a gene of interest. The expression of the gene of interest may be manipulated by ASOs to inhibit splicing of an exon, intron or a specific splice site of a gene of interest, leading to, e.g., without limitation, restoring the reading frame of a defective gene of interest, generating a different isoform of a gene of interest (e.g., a dominant negative isoform), skipping a toxic part of the gene, silencing the gene, and/or changing the structure and function of a gene.

some aspects, the present technology provides a use of the ASO (e.g., bipartite ASO) disclosed herein, the composition (e.g., pharmaceutical composition) comprising the ASO (e.g., bipartite ASO) as disclosed herein, and/or the vector encoding the ASO (e.g., bipartite ASO) as disclosed herein to generate or promote exon skipping of an exon of interest. The ASO-mediated exon skipping may be an approach to manipulate expression of a gene of interest. The expression of the gene of interest may be manipulated by ASOs to inhibit splicing of an exon, intron or a specific splice site of a gene of interest, leading to, e.g., without limitation, restoring the reading frame of a defective gene of interest, generating a different isoform of a gene of interest (e.g., a dominant negative isoform), skipping a toxic part of the gene, silencing the gene, and/or changing the structure and function of a gene.

In some aspects, the present technology provides the ASO (e.g., bipartite ASO) disclosed herein, the composition (e.g., pharmaceutical composition) comprising the ASO (e.g., bipartite ASO) as disclosed herein, and/or the vector encoding the ASO (e.g., bipartite ASO) as disclosed herein for use in generating or promoting exon skipping of an exon of interest. The ASO-mediated exon skipping may be an approach to manipulate expression of a gene of interest. The expression of the gene of interest may be manipulated by ASOs to inhibit splicing of an exon, intron or a specific splice site of a gene of interest, leading to, e.g., without limitation, restoring the reading frame of a defective gene of interest, generating a different isoform of a gene of interest (e.g., a dominant negative isoform), skipping a toxic part of the gene, silencing the gene, and/or changing the structure and function of a gene.

An ASO comprises a targeting sequence which is a single-stranded oligonucleotide that is specific for, and substantially complementary to, a splicing sequence of interest, and accordingly is capable of hydrogen bonding to the sequence. One of skill in the art can readily design the targeting sequence of an ASO to be specific for suitable targeted regions, many of which are well-known in the art. For example, one can access pre-mRNA sequences comprising suitable splicing sequences in publications or in annotated, publicly available databases, such as the GenBank database operated by the NCBI. A number of targeting sequences have been incorporated in the design of ASOs for enhancing exon skipping and some are currently in preclinical or clinical trials. Any of these targeting sequences is suitable for use in a method of the present technology.

ASOs can be used to modulate exon skipping by blocking (hiding) specific sequence motifs in the pre-mRNA (sometimes referred to herein as “splicing sequences”) essential for exon inclusion from the splicing machinery. ASOs that block aberrant splice sites can restore normal splicing. Alternatively, ASOs targeting some splicing sequences can switch splicing patterns from detrimental to beneficial isoforms or can convert non-functional mRNAs into at least partially functional mRNA. An example of the latter approach is the restoration of a disrupted reading frame, thereby generating semi-functional proteins instead of non-functional proteins.

An ASO of the present technology (e.g., bipartite ASOs) can be used to block splicing at a site of interest by specifically interacting with (e.g., binding to) a splicing sequence at that site, cither directly or indirectly. By a “splicing sequence” is meant a sequence that regulates and/or is required for splicing out of a particular intron and/or the retention of a particular exon. The splicing sequence can be, for example, a splice donor site (5′ splice site), a splice acceptor site (3′ splice site), a branch site, an intronic splicing enhancer (ISE), an exonic splicing enhancer (ESE), an intronic splicing silencer (ISS) or an exonic splicing silencer (ESS).

An ASO used in a method of the present technology (e.g., bipartite ASOs) can bind directly and specifically to a target region, which is a target splicing sequence of interest. By “specific binding” is meant that the ASO binds preferentially to the targeted region, but not to non-targeted sequences under conditions in which specific binding is desired. The conditions can be, e.g., physiological conditions in the case of in vivo assays or therapeutic treatment, and for in vitro assays, conditions in which the assays are performed. Because the mechanism by which small molecule compounds of the present technology block splicing (e.g., enhance exon skipping) is not known for all of the compounds, it is not known whether the compound binds directly to a splice site or acts indirectly (e.g., by binding to another RNA or protein element of a spliceosome). Regardless of the mechanism, a compound of the present technology that “specifically” blocks a splicing event of interest is one that preferentially blocks the particular splicing event but does not block non-targeted splicing events, under conditions in which specific blocking is desired.

ASOs (e.g., bipartite ASOs) disclosed herein may have a variety of different backbone chemistries, such as morpholino phosphorodiamidate (PMO), 2′-O-methyl, 2′-O-methoxyethyl (MOE), phosphorothioates (PS), 2′-fluoro ribose (2′-F), 4′-thioribosyl ribose, locked nucleic acid oligos (LNA), and/or constrained ethyl oligos (cEt), or peptide nucleic acids, etc., which stabilize them. For example, it can be DNA, RNA, PNA or LNA, or chimeric mixtures or derivatives or modified versions thereof. The nucleic acid can be modified at the base moiety, sugar moiety, or phosphate backbone, using conventional procedures and modifications. Modifications of the bases include, e.g., methylated versions of purines or pyrimidines. Modifications may include other appending groups that will be evident to a skilled worker. Examples of oligonucleotide modifications are described throughout the instant application, for example, in the “oligonucleotide modification” section below.

ASOs (e.g., bipartite ASOs) disclosed herein can be constructed using chemical synthesis procedures known in the art. An ASO can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Alternatively, an ASO can be produced biologically using an expression vector into which a nucleic acid has been subcloned in an antisense orientation (i.e., nucleic acid transcribed from the inserted nucleic acid will be of an antisense orientation to a targeted region). Expression control sequences (e.g., regulatory sequences) operatively linked to a nucleic acid cloned in the antisense orientation can be chosen which direct the expression of the antisense RNA molecule in a cell of interest. For instance, promoters and/or enhancers or other regulatory sequences can be chosen which direct constitutive, tissue specific or inducible expression of an ASO. Inducible expression of antisense RNA, e.g., regulated by an inducible eukaryotic regulatory system, can be used. The antisense expression vector can be in the form of, for example, a recombinant plasmid, phagemid or attenuated virus. Suitable viral vectors include, e.g., adeno-associated virus (AAV) or lentivirus vectors. The antisense expression vector can be introduced into cells using standard techniques well known in the art.

The length of targeting sequence within an ASO may vary, provided that it is capable of binding selectively to the intended splicing sequence within the pre-mRNA molecule. A skilled worker can readily determine a satisfactory length. Generally, the targeting sequence of an ASO is from about 10 nt in length to about 80 nt in length. Any length of nucleotides within this range, including the endpoints, can be used in a method of the present technology.

In some embodiments of the present technology, the targeting sequence of the ASO comprises a strand that is fully (100%) complementary to a splicing sequence that it is designed to inhibit. That is, every contiguous nucleotide in the targeting sequence will hybridize to every nucleotide in a splicing sequence in the target gene. However, 100% sequence identity between the targeting sequence and the targeted region is not required. Thus, the present technology has the advantage of being able to tolerate naturally occurring sequence variations that might be expected due to genetic mutation, strain polymorphism, or evolutionary divergence. Alternatively, the variants may be artificially generated. Nucleic acid sequences with, e.g., small insertions, deletions, and single point mutations relative to the targeted region can be effective for inhibition. The degree of sequence identity can be, e.g., 90%, 95%, 98%, 99%, or 100%. Such a variant ASO must, of course, retain the relevant activity of the ASO from which it is derived (e.g., the ability to suppress splicing at a site of interest). Such variants are sometimes referred to herein as “active variants.”

For further guidance for designing suitable antisense molecules that are complementary to a region of a pre-mRNA involved in splicing (thereby blocking splicing), and for methods for making and delivering such molecules to a cell or a subject, see, e.g., US 2008/0200409 or U.S. Pat. Nos. 7,973,015, 7,960,541, 7,902,160, 7,888,012, 7,879,992 or 7,737,110.

### Examples of Oligonucleotides for Dystrophin Gene Splicing Modulation

In September 2016, the US Food and Drug Administration (FDA) conditionally approved the first DMD antisense drug, eteplirsen (Exondys 51), which was developed to exclude exon 51 from mutant DMD. Eteplirsen is an oligonucleotide modified with a phosphorodiamidate morpholino oligomer (morpholino or PMO). However, eteplirsen remains controversial as there is only weak evidence supporting the effectiveness of the drug, both in terms of restoring dystrophin protein to therapeutically beneficial levels, and improving clinical outcomes. The FDA has previously rejected another drug candidate for DMD exon 51 skipping: the 2′-O-methyl-phosphorothioate-based oligonucleotide “drisapersen.” Although therapeutics must ensure the highest possible benefit for the lowest amount of risk, no significant improvements in muscle function were demonstrated upon treatment with drisapersen, and its use led to concerns over safety.

### Bipartite Antisense Oligonucleotides (Bipartite ASOs)

In some aspects, the present technology provides bipartite ASOs comprising: 1) a “targeting” sequence that is fully or substantially complementary to the targeted region in a target nucleic acid (e.g., pre-mRNA of a gene of interest), and 2) a “decoy” sequence that is located operably connected at the 5′ end and/or the 3′ end of the targeting sequence. The decoy sequence may simulate an optimal 5′ splice site, and thus it may serve as a 5′-splice-site decoy to interfere with the recognition of an adjacent authentic 5′ splice site by U1 snRNA. As discussed herein, the presence of the decoy sequence may increase the efficiency of splicing modulation at the targeted region by the targeting sequence.

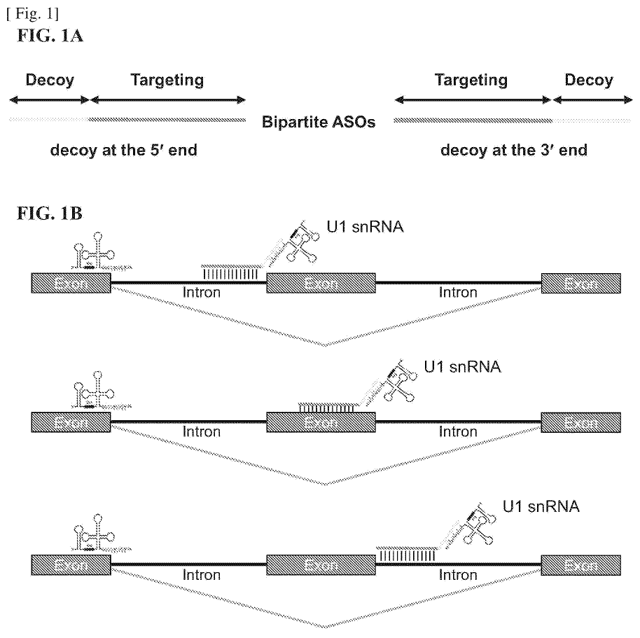

In various embodiments, bipartite ASOs of the present technology comprise: 1) a “targeting” sequence that is fully or substantially complementary to the targeted region in the target nucleic acid (e.g., pre-mRNA of a gene of interest), and 2) a “decoy” sequence that is located immediately upstream from the 5′ end and/or immediately downstream from the 3′ end of the targeting sequence (i.e., the decoy sequence is in the flanking region of the targeting sequence). In some embodiments, the decoy sequence is directly connected to the targeting sequence with no intermediate nucleotides between the decoy sequence and the targeting sequence. In some embodiments, the decoy sequence is located immediately upstream from the 5′ end of the targeting sequence (as shown in FIG. 1A). In other embodiments, the decoy sequence is located immediately downstream from the 3′ end of the targeting sequence.

When both the 5′ and 3′ ends of the targeting sequence are connected with a decoy sequence, the decoy sequences connected to each end may be the same or different, and the manner of connection may be the same of different. For example, both decoy sequences may be connected to the targeting sequence without a linker, or one decoy sequence is connected without a linker while the other decoy sequence is connected with a linker, or both decoy sequences are connected to the targeting sequence with a linker but the linker may be the same or different. In certain embodiments, a linker may have 1, 2, 3, 4, or 5 nucleotides.

In some embodiments, ASOs comprising the same targeting sequence may have different exon skipping effects and/or efficiencies due to the presence of the decoy sequence (e.g., with or without the decoy sequence), the length of the decoy sequence, the sequence of the decoy sequence, and/or the position of the decoy sequence (e.g., 5′ and/or 3′ ends of the targeting sequence). In certain embodiments, the exon skipping effects and/or efficiencies may be quantified by the percentage of exclusion of the exon of interest (% excl) in total transcripts of each gene. In certain embodiments, the improvement as quantified by increase of % excl may be at least about 2 fold, at least about 2.18 fold, at least about 2.3 fold, at least about 3 fold, at least about 4 fold, at least about 5 fold, at least about 6 fold, at least about 7 fold, at least about 8 fold, at least about 9 fold, at least about 10 fold, at least about 11 fold, at least about 12 fold, at least about 13 fold, at least about 14 fold, at least about 15 fold, at least about 16 fold, at least about 17 fold, at least about 18 fold, at least about 19 fold, at least about 20 fold, at least about 25 fold at least about 30 fold, at least about 40 fold, at least about 50 fold, at least about 60 fold, at least about 70 fold, at least about 80 fold, at least about 90 fold, and at least about 100 fold, including all values and ranges between these values, of that of the ASO consisting of the targeting sequence.

In certain embodiments, a bipartite ASO comprising or consisting of a decoy sequence (e.g., without limitation, the nucleotide sequences of SEQ ID Nos. 1 to 23 and 347 to 353) attached to a targeting sequence may achieve a comparable efficacy and/or efficiency in generating or promoting exon skipping at a lower dose than an ASO consisting of the targeting sequence. In certain embodiments, an ASO comprising a decoy sequence attached to a targeting sequence may achieve a comparable (e.g., at least about 70%, at least about 80%, at least about 90%, at least about 100%, at least about 110%, at least about 120%, at least about 130%, at least about 140%, at least about 150%) % excl of an ASO consisting of the targeting sequence at a dose of no more than about 10%, no more than about 25%, no more than about 40%, or no more than about 50% of the dose of the ASO consisting of the targeting sequence.

In certain embodiments of the bipartite ASOs comprising the same targeting sequence and decoy sequence, the bipartite ASO having the decoy sequence at the 5′ end of the targeting sequence may have higher exon skipping effects and/or efficiencies than the bipartite ASO having the decoy sequence at the 3′ end of the targeting sequence. In certain examples, both bipartite ASOs comprising the decoy sequence have higher exon skipping effects and/or efficiencies than the ASO consisting of the same targeting sequence.

In certain embodiments of the bipartite ASOs comprising the same targeting sequence and decoy sequence, the bipartite ASO having the decoy sequence at the 5′ end of the targeting sequence may have lower exon skipping effects and/or efficiencies than the bipartite ASO having the decoy sequence at the 3′ end of the targeting sequence. In certain examples, both bipartite ASOs comprising the decoy sequence have higher exon skipping effects and/or efficiencies than the ASO consisting of the same targeting sequence.

In certain embodiments of the ASOs comprising the same targeting sequence and decoy sequence, the ASO having the decoy sequence at the 5′ end of the targeting sequence and the ASO having the decoy sequence at the 3′ end of the targeting sequence are comparable.

In some embodiments the bipartite ASO comprises or consists of a nucleotide sequence selected from the group consisting of nucleotide sequences of SEQ ID Nos. 26 to 27, 30 to 31, 34 to 35, 38 to 39, 42 to 43, 46 to 47, 50 to 73, 76 to 90, 93 to 122, 125 to 154, 157 to 186, 189 to 218, 221 to 250, 253 to 282, 285 to 314, 317 to 346, and 354 to 356. In some embodiments, the bipartite ASO comprises or consists of a nucleotide sequence selected from the group consisting of nucleotide sequences of SEQ ID Nos. 26, 30, 31, 34, 38, 42, 46, 50, 51, 60, 64, 65, 68, 70, 78, 83, 85, 86, 87, 88, 89, 103, 109, 110, 114, 115, 116, 127, 132, 144, 164, 189, 205, 213, 216, 228, 260, 276, 292, 294, 297, 298, 312, 320, 324, 327, 328, 332, and 354.

In some embodiments, the gene of interest is SMN1 and/or SMN2 gene, the ASO comprises or consists of a nucleotide sequence selected from the group consisting of nucleotide sequences of SEQ ID Nos. 26 to 27, 30 to 31, 34 to 35, and 354 to 356. In certain embodiments, the ASO comprises or consists of a nucleotide sequence selected from the group consisting of nucleotide sequences of SEQ ID Nos. 26, 30, 31, 34, and 354. In certain embodiments, the exon of interest is exon 7 of SMN1 and/or SMN2 gene.

In some embodiments, the gene of interest is DMD gene, the ASO comprises or consists of a nucleotide sequence selected from the group consisting of nucleotide sequences of SEQ ID Nos. 38 to 39, 42 to 43, 46 to 47, 50 to 73, 76 to 90, 93 to 122, and 125 to 154. In certain embodiments, the optimal ASO comprises or consists of a nucleotide sequence selected from the group consisting of nucleotide sequences of SEQ ID Nos. 38, 42, 46, 50, 51, 60, 64, 65, 68, 70, 78, 83, 85, 86, 87, 88, 89, 103, 109, 110, 114, 115, 116, 127, 132, and 144.

In certain embodiments, the exon of interest is exon 51 of DMD gene, the ASO comprises or consists of a nucleotide sequence selected from the group consisting of nucleotide sequences of SEQ ID Nos. 38 to 39, 42 to 43, 46 to 47, 50 to 73, and 76 to 90. In certain embodiments, the optimal ASO comprises or consists of a nucleotide sequence selected from the group consisting of nucleotide sequences of SEQ ID Nos. 38, 42, 46, 50, 51, 60, 64, 65, 68, 70, 78, 83, 85, 86, 87, 88, and 89.

In certain embodiments, the exon of interest is exon 53 of DMD gene, the ASO comprises or consists of a nucleotide sequence selected from the group consisting of nucleotide sequences of SEQ ID Nos. 93 to 122. In certain embodiments, the optimal ASO comprises or consists of a nucleotide sequence selected from the group consisting of nucleotide sequences of SEQ ID Nos. 103, 109, 110, 114, 115, and 116.

In certain embodiments, the exon of interest is exon 45 of DMD gene, the ASO comprises or consists of a nucleotide sequence selected from the group consisting of nucleotide sequences of SEQ ID Nos. 125 to 154. In certain embodiments, the optimal ASO comprises or consists of a nucleotide sequence selected from the group consisting of nucleotide sequences of SEQ ID Nos. 127, 132, and 144.

In some embodiments, the gene of interest is APP gene, the ASO comprises or consists of a nucleotide sequence selected from the group consisting of nucleotide sequences of SEQ ID Nos. 157 to 186. In certain embodiments, the optimal ASO comprises or consists of a nucleotide sequence selected from the group consisting of nucleotide sequences of SEQ ID No. 164. In certain embodiments, the exon of interest is exon 17 of APP gene.

In some embodiments, the gene of interest is CEP290 gene, the ASO comprises or consists of a nucleotide sequence selected from the group consisting of nucleotide sequences of SEQ ID Nos. 189 to 218. In certain embodiments, the optimal ASO comprises or consists of a nucleotide sequence selected from the group consisting of nucleotide sequences of SEQ ID Nos. 189, 205, 213, and 216. In certain embodiments, the exon of interest is exon 41 of CEP290 gene.

In some embodiments, the gene of interest is HER2 gene, the ASO comprises or consists of a nucleotide sequence selected from the group consisting of nucleotide sequences of SEQ ID Nos. 221 to 250. In certain embodiments, the optimal ASO comprises or consists of a nucleotide sequence selected from the group consisting of nucleotide sequences of SEQ ID No. 228. In certain embodiments, the exon of interest is exon 19 of HER2 gene.

In some embodiments, the gene of interest is ATXN3 gene, the ASO comprises or consists of a nucleotide sequence selected from the group consisting of nucleotide sequences of SEQ ID Nos. 253 to 282. In certain embodiments, the optimal ASO comprises or consists of a nucleotide sequence selected from the group consisting of nucleotide sequences of SEQ ID Nos. 260 and 276. In certain embodiments, the exon of interest is exon 10 of ATXN3 gene.

In some embodiments, the gene of interest is PKM gene, the ASO comprises or consists of a nucleotide sequence selected from the group consisting of nucleotide sequences of SEQ ID Nos. 285 to 314. In certain embodiments, the optimal ASO comprises or consists of a nucleotide sequence selected from the group consisting of nucleotide sequences of SEQ ID Nos. 292, 294, 297, 298, and 312. In certain embodiments, the exon of interest is exon 10 of PKM gene.

In some embodiments, the gene of interest is MDM4 gene, the ASO comprises or consists of a nucleotide sequence selected from the group consisting of nucleotide sequences of SEQ ID Nos. 317 to 346. In certain embodiments, the optimal ASO comprises or consists of a nucleotide sequence selected from the group consisting of nucleotide sequences of SEQ ID Nos. 320, 324, 327, 328, and 332. In certain embodiments, the exon of interest is exon 6 of MDM4 gene. Decoy sequences

In some embodiments, the decoy sequence comprises or consists of a nucleotide sequence selected from the group consisting of nucleotide sequences of SEQ ID Nos. 1, 2, 3, 4, 5, 6, 7, 8, 9, 10, 11, 12, 13, 14, 15, 16, 17, 18, 19, 20, 21, 22, 23, 347, 348, 349, 350, 351, 352, and 353. In some embodiments, the length of the decoy sequence is from 5 to 13 nucleotides (nt). In some embodiments, the length of the decoy sequence is 5, 6, 7, 8, 9, 10, 11, 12, or 13 nt.

In some embodiments, the decoy sequence resembles an optimal 5′ splice site and is fully or partially complementary to the single-strand 5′ end of U1 snRNA. In some embodiments, the decoy sequence is 100% complementary to a portion of the single-strand 5′ end of U1 snRNA. In some embodiments, 7-11 nt in the decoy sequence are complementary to the 5′ end of U1 snRNA.

In some embodiments, the decoy sequence has low sequence complementarity, such as less than about 80%, to the corresponding flanking sequence immediately adjacent to the 5′ or 3′ end of the targeted region in the target nucleic acid (e.g., mRNA). In some embodiments, the decoy sequence is located immediately upstream from the 5′ end of the targeting sequence, and has low sequence complementarity to the corresponding flanking region immediately downstream from the 3′ end of the targeted region (as shown in FIG. 1A). In some embodiments, the decoy sequence is located immediately downstream from the 3′ end of the targeting sequence, and has low sequence complementarity to the corresponding flanking region immediately upstream from the 5′ end of the targeted region in the target nucleic acid (such as a pre-RNA, and as shown in FIG. 1A). In some embodiments, the decoy sequence has less than about 80% sequence complementarity to the corresponding region in the target nucleic acid. In some embodiments, the decoy sequence has less than about 70% sequence complementarity to the corresponding region in the target nucleic acid. In some embodiments, the decoy sequence has less than about 60% sequence complementarity to the corresponding region in the target nucleic acid. In some embodiments, the decoy sequence has less than about 50% sequence complementarity to the corresponding region in the target nucleic acid. In some embodiments, said sequence complementarity is less than about 40%, less than about 35%, less than about 30%, less than about 25%, less than about 20%, less than about 15%, less than about 10%, less than about 5%, or 0%, to the corresponding region in the target nucleic acid.

In various embodiments, the first nucleotide of the decoy sequence that is immediately adjacent to the 5′ or 3′ end of the targeting sequence is not complementary to the corresponding nucleotide flanking the targeted region in the target nucleic acid. In some embodiments, the decoy sequence is located upstream from the 5′ end of the targeting sequence in the ASO, and in such embodiments the first nucleotide at the 3′ end of the decoy sequence is not complementary to the corresponding flanking nucleotide located immediately downstream from the 3′ end of the targeted region in the target nucleic acid (as shown in FIG. 1A). In some embodiments, the decoy sequence is located downstream from the 3′ end of the targeting sequence in the ASO, and in such embodiments the first nucleotide at the 5′ end of the decoy sequence is not complementary to the corresponding flanking nucleotide located immediately upstream from the 5′ end of the targeted region in the target nucleic acid (as shown in FIG. 1A).

### Targeting Sequences

In various embodiments of the present technology, the targeting sequence of the ASO is fully or substantially complementary to the targeted region in the nucleic acid (e.g., pre-mRNA). In some embodiments, the targeting sequence of the ASO comprises a strand that has exact (100%) complementarity to the targeted region in the nucleic acid that it is designed to inhibit. That is, every contiguous nucleotide in the targeting sequence is hybridized to every corresponding nucleotide in the targeted region. However, 100% sequence identity between the targeting sequence and the targeted splicing sequence may not be required to practice the present technology. In other embodiments, the targeting sequence is substantially complementary to the targeted region; i.e., the targeting sequence is more than 70%, more than 75%, more than 80%, more than 90%, more than 95%, more than 98%, or more than 99%, complementary to the targeted region in the nucleic acid that it is designed to inhibit. The non-complementary position(s) in the targeting sequence of ASO maybe one or more insertions, deletions, and/or point mutations relative to the targeted region.

In some embodiments, the targeted region is an exon (as shown in, for example, FIG. 1B). In some embodiments, the targeted region is an intron region upstream from an exon (as shown in FIG. 1B). In some embodiments, the targeted region is an intron region downstream from an exon (as shown in FIG. 1B). In some embodiments the targeting sequence binds to the targeted region which is an exon of interest, a flanking intron sequence upstream of the exon of interest, a flanking intron sequence downstream of the exon of interest, an intron-exon junction upstream of the exon of interest, or an intron-exon junction downstream of the exon of interest. In some embodiments, the exon of interest is exon 7 of the SMN1 gene or SMN2 gene. In some embodiments, the exon of interest is exon 7 of the SMN1 gene. In some embodiments, the exon of interest is exon 7 of the SMN2 gene. In some embodiments, the exon of interest is exon 51 of the DMD gene. In some embodiments, the exon of interest is exon 53 of the DMD gene. In some embodiments, the exon of interest is exon 45 of the DMD gene. In some embodiments, the exon of interest is exon 17 of the APP gene. In some embodiments, the exon of interest is exon 41 of the CEP290 gene. In some embodiments, the exon of interest is exon 19 of the HER2 gene. In some embodiments, the exon of interest is exon 10 of the ATXN3 gene. In some embodiments, the exon of interest is exon 10 of the PKM gene. In some embodiments, the exon of interest is exon 6 of the MDM4 gene.

In various embodiments, the length of the targeting sequence of ASO is from about 10 nt in length to about 80 nt in length. In some embodiments, the length of the targeting sequence of ASO is about 10-60 nt in length. In some embodiments, the length of the targeting sequence of ASO is about 10-50 nt in length. In some embodiments, the length of the targeting sequence of ASO is about 10-40 nt in length. In some embodiments, the length of the targeting sequence of ASO is about 12-35 nt in length. In some embodiments, the length of the targeting sequence of ASO is about 14-30 nt in length. In some embodiments, the length of the targeting sequence of ASO is about 15-25 nt in length. In some embodiments, the length of the targeting sequence of ASO is about 18-23 nt in length. In some embodiments, the length of the targeting sequence of ASO is about 20 nt in length. In some embodiments, the targeting sequence of ASO comprises or consists of a nucleotide sequence selected from the group consisting of nucleotide sequences of SEQ ID Nos. 25, 29, 33, 37, 41, 45, 49, 75, 92, 124, 156, 188, 220, 252, 284, and 316.

In some embodiments, the targeting sequence hybridizes to SMN1 and/or SMN2 gene comprises or consists of a nucleotide sequence selected from the group consisting of nucleotide sequences of SEQ ID Nos. 25, 29, and 33. In certain embodiments, the targeting sequence is for exon skipping of exon 7 of SMN1 and/or SMN2 gene.

In some embodiments, the targeting sequence hybridizes to DMD gene comprises or consists of a nucleotide sequence selected from the group consisting of nucleotide sequences of SEQ ID Nos. 37, 41, 45, 49, 75, 92, and 124.

In certain embodiments, the targeting sequence is for exon skipping of exon 51 of DMD gene, and the targeting sequence hybridizes to DMD gene comprises or consists of a nucleotide sequence selected from the group consisting of nucleotide sequences of SEQ ID Nos. 37, 41, 45, 49, and 75.

In certain embodiments, the targeting sequence is for exon skipping of exon 53 of DMD gene, and the targeting sequence hybridizes to DMD gene comprises or consists of a nucleotide sequence selected from the group consisting of nucleotide sequences of SEQ ID No. 92.

In certain embodiments, the targeting sequence is for exon skipping of exon 45 of DMD gene, and the targeting sequence hybridizes to DMD gene comprises or consists of a nucleotide sequence selected from the group consisting of nucleotide sequences of SEQ ID No. 124.

In some embodiments, the targeting sequence hybridizes to APP gene comprises or consists of a nucleotide sequence selected from the group consisting of nucleotide sequences of SEQ ID No. 156. In certain embodiments, the targeting sequence is for exon skipping of exon 17 of APP gene.

In some embodiments, the targeting sequence hybridizes to CEP290 gene comprises or consists of a nucleotide sequence selected from the group consisting of nucleotide sequences of SEQ ID No. 188. In certain embodiments, the targeting sequence is for exon skipping of exon 41 of CEP290 gene.

In some embodiments, the targeting sequence hybridizes to HER2 gene comprises or consists of a nucleotide sequence selected from the group consisting of nucleotide sequences of SEQ ID No. 220. In certain embodiments, the targeting sequence is for exon skipping of exon 19 of HER2 gene.

In some embodiments, the targeting sequence hybridizes to ATXN3 gene comprises or consists of a nucleotide sequence selected from the group consisting of nucleotide sequences of SEQ ID No. 259. In certain embodiments, the targeting sequence is for exon skipping of exon 10 of ATXN3 gene.

In some embodiments, the targeting sequence hybridizes to PKM gene comprises or consists of a nucleotide sequence selected from the group consisting of nucleotide sequences of SEQ ID No. 284. In certain embodiments, the targeting sequence is for exon skipping of exon 10 of PKM gene.

In some embodiments, the targeting sequence hybridizes to MDM4 gene comprises or consists of a nucleotide sequence selected from the group consisting of nucleotide sequences of SEQ ID No. 316. In certain embodiments, the targeting sequence is for exon skipping of exon 6 of MDM4 gene.

### Modifications

In some embodiments, additional flanking nucleotide sequence may be present at one or both termini of the oligonucleotide comprising the decoy sequence and the targeting sequence.

In some embodiments, the ASO further comprises one or more nucleotide modifications. In some embodiments, at least one subunit of the ASO is a non-natural nucleotide analog having (i) a modified inter-nucleoside linkage, (ii) a modified sugar moiety, (iii) a modified base, or (iv) a combination of the foregoing.

In some embodiments, the ASO comprises one or more phosphorothioate linkages as the modified inter-nucleoside linkages. In some embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more inter-nucleoside linkages of the ASO are phosphorothioate linkages. In some embodiments, all inter-nucleoside linkages of the ASO are phosphorothioate linkages.

In some embodiments, the ASO comprises one or more 2′-O-methoxyethyl sugar moieties as the modified sugar moieties. In some embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more sugar moieties of the ASO are 2′-O-methoxyethyl sugar moieties. In some embodiments, all sugar moieties of the ASO are 2′-O-methoxyethyl sugar moieties.

In some embodiments, the ASO comprises one or more 5-methylcytosines in place of cytosines as the modified bases. In some embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more cytosine bases of the ASO are modified as 5-methylcytosines. In some embodiments, all cytosine bases of the ASO are modified as 5-methylcytosines.

In some embodiments, the ASO further comprises one or more additional chemical moieties. In some embodiments, one or more additional chemical moieties are covalently linked to one or more termini of the nucleic acid sequence. In some embodiments, the additional chemical moiety is a cell penetrating peptide.

In some embodiments, the ASO of the present technology may include a nucleic acid moiety conjugated to a cell penetrating peptide (CPP) moiety to enhance transport of the compound into cells. In some embodiments, the CPP moiety is attached to a terminus of the oligonucleotide. In some embodiments, the peptides have the capability of penetrating about 10%, 20%, 30%, 40%, 50%, 60%, 70%, 80%, 90% or 100% of cells of a given cell culture population, including all integers in between, and allow macromolecular translocation within multiple tissues in vivo upon administration. In some embodiments, the cell-penetrating peptide may be an arginine-rich peptide transporter. In some embodiments, the cell-penetrating peptide may be Penetratin or the Tat peptide. These peptides are well known in the art and are disclosed, for example, in US Publication No. 2010-0016215 A1, incorporated by reference in its entirety. An exemplar approach to conjugation of peptides to ASO can be found in PCT publication WO2012/150960, which is incorporated by reference in its entirety. In some embodiments, the oligonucleotide and the CPP moiety are conjugated through a linker. In some embodiments, amino acid glycine is used the linker between the CPP and the oligonucleotide moiety.

The transport moieties as described above have been shown to greatly enhance cell entry of attached oligomers, relative to uptake of the oligomer in the absence of the attached transport moiety. In some embodiments, the CPP is one of the arginine-rich cell penetrating peptides. Some peptide transporters have been shown to be highly effective at delivery of compounds comprising antisense sequences into primary cells including muscle cells (N. B. Marshall, et al. Arginine-rich cell-penetrating peptides facilitate delivery of antisense oligomers into murine leukocytes and alter pre-mRNA splicing. 2007 Aug. 31. Journal of Immunological Methods. 325:114-126. doi: 10.1016/j. jim. 2007.06.009; N. Jearawiriyapaisarn, et al. Sustained dystrophin expression induced by peptide-conjugated morpholino oligomers in the muscles of mdx micc. 2008 September. Mol Ther. 16:1624-1629. doi: 10.1038/mt. 2008.120; Wu B, et al. Effective rescue of dystrophin improves cardiac function in dystrophin-deficient mice by a modified morpholino oligomer. 2008 Sep. 30. Proc. Natl. Acad. Sci. USA. 105:14814-14819. doi: 10.1073/pnas. 0805676105).

### Assays to Identify Bipartite ASOs for Exon-Skipping Effects

A non-limiting example of determining whether an ASO induces skipping of one or more exons in a transcript of a gene of interest can be the following: introducing the ASOs to be tested into a suitable cell line (e.g., HEK293 cell for SMN1, SMN2, CEP290, and MDM4 genes, HeLa cell for HER2 gene, A549 cell for ATXN3 gene, a dystrophin expression cell such as human rhabdomyosarcoma cells for DMD and PKM genes, or a cell line that express a gene construct that mimics the exon/intron organization of the region of interest (e.g., by introducing an artificial minigene containing plasmid into a cell line), and amplifying the region encompassing the exon of interest in the transcript from the total RNA of the cell line by RT-PCR and performing nested PCR or sequence analysis on the PCR amplified product.

The skipping efficiency can be determined as follows. The mRNA of dystrophin gene is collected from test cells and amplified by RT-PCR. If we denote “A” as the level of amplified polynucleotide of our target mRNA in which one or more exons of interest are skipped, and if we further denote “B” as the level of amplified polynucleotide of our target mRNA where the exon(s) of interest are not skipped, then using these measurement values of “A” and “B,” the efficiency is calculated by the following equation:

Exon skipping rate/efficiency (% excl)=A/(A+B)×100

Exon inclusion rate (% incl)=B/(A+B)×100

In some embodiments, the bipartite ASOs of the present technology cause skipping of one or more of exons in DMD mRNA with the exon skipping percentage (% excl) increased by about 10% or higher, about 20% or higher, about 30% or higher, about 40% or higher, about 50% or higher, about 60% or higher, about 70% or higher, about 80% or higher, about 90% or higher, about 2 fold or higher, about 2.18 fold or higher, about 2.3 fold or higher, about 3 fold or higher, about 4 fold or higher, about 5 fold or higher, about 6 fold or higher, about 7 fold or higher, about 8 fold or higher, about 9 fold or higher, about 10 fold or higher, about 11 fold or higher, about 12 fold or higher, about 13 fold or higher, about 14 fold or higher, about 15 fold or higher, about 16 fold or higher or higher, about 17 fold or higher, about 18 fold or higher, about 19 fold or higher, about 20 fold or higher, about 25 fold or higher, about 30 fold or higher, about 40 fold or higher, about 50 fold or higher, about 60 fold or higher, about 70 fold or higher, about 80 fold or higher, about 90 fold or higher, about 100 fold or higher, including all values and ranges between these values, than the ASO having the same targeting sequence but without a decoy.

### Oligonucleotide Modifications

Unmodified oligonucleotides may be less than optimal in some applications, e.g., unmodified oligonucleotides can be prone to degradation by e.g., cellular nucleases. Nucleases can hydrolyze nucleic acid phosphodiester bonds. However, chemical modifications of oligonucleotides can confer improved properties, and, e.g., can render oligonucleotides more stable to nucleases.

As oligonucleotides are polymers of subunits or monomers, many of the modifications described below occur at a position which is repeated within an oligonucleotide, e.g., a modification of a base, a sugar, a phosphate moiety, or the non-bridging oxygen of a phosphate moiety. It is not necessary for all positions in a given oligonucleotide to be uniformly modified, and in fact more than one of the aforementioned modifications may be incorporated in a single oligonucleotide or even at a single nucleoside within an oligonucleotide.

In some embodiments, the modification will occur at all of the subject positions in the oligonucleotide but in many embodiments it will not. By way of example, a modification may only occur at a 3′ or 5′ terminal position, may only occur in the internal region, may only occur in a terminal regions, e.g., at a position on a terminal nucleotide or in the last 2, 3, 4, 5, or 10 nucleotides of an oligonucleotide. A modification may occur in a double strand region, a single strand region, or in both. A modification may occur only in the double strand region of a double-stranded oligonucleotide or may only occur in a single strand region of a double-stranded oligonucleotide. E.g., a phosphorothioate modification at a non-bridging oxygen position may only occur at one or both termini, may only occur in a terminal regions, e.g., at a position on a terminal nucleotide or in the last 2, 3, 4, 5, or 10 nucleotides of a strand, or may occur in double strand and single strand regions, particularly at termini. The 5′ end or ends can be phosphorylated.

A modification described herein may be the sole modification, or the sole type of modification included on multiple nucleotides, or a modification can be combined with one or more other modifications described herein. The modifications described herein can also be combined onto an oligonucleotide, e.g., different nucleotides of an oligonucleotide have different modifications described herein.

In some embodiments, it may be desirable, e.g., to enhance stability, to include particular nucleobases in overhangs, or to include modified nucleotides or nucleotide surrogates, in single strand overhangs, e.g., in a 5′ or 3′ overhang, or in both. E.g., it can be desirable to include purine nucleotides in overhangs. In some embodiments all or some of the bases in a 3′ or 5′ overhang will be modified, e.g., with a modification described herein. Modifications can include, e.g., the use of modifications at the 2′OH group of the ribose sugar, e.g., the use of deoxyribonucleotides, e.g., deoxythymidine, instead of ribonucleotides, and modifications in the phosphate group, e.g., phosphothioate modifications. Overhangs need not be homologous with the targeted region.

Specific modifications are discussed in more detail below.

### The Phosphate Group

The phosphate group is a negatively charged species. The charge is distributed equally over the two non-bridging oxygen atoms. However, the phosphate group can be modified by replacing one of the oxygens with a different substituent. One result of this modification to RNA phosphate backbones can be increased resistance of the oligoribonucleotide to nucleolytic breakdown. Thus while not wishing to be bound by theory, it can be desirable in some embodiments to introduce alterations which result in either an uncharged linker or a charged linker with unsymmetrical charge distribution. Examples of modified phosphate groups include phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the phosphate backbone moiety can be replaced by any of the following: S, Se, BR3 (R is hydrogen, alkyl, aryl), C (i.e. an alkyl group, an aryl group, etc.), H, NR2 (R is hydrogen, alkyl, aryl), or (R is alkyl or aryl). The phosphorous atom in an unmodified phosphate group is achiral. However, replacement of one of the non-bridging oxygens with one of the above atoms or groups of atoms renders the phosphorous atom chiral; in other words a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligoribonucleotides diastereomers. Thus, while not wishing to be bound by theory, modifications to both non-bridging oxygens, which eliminate the chiral center, e.g., phosphorodithioate formation, may be desirable in that they cannot produce diastereomer mixtures. Thus, the non-bridging oxygens can be independently any one of S, Sc, B, C, H, N, or (R is alkyl or aryl).

The phosphate linker can also be modified by replacement of bridging oxygen, (i.e. oxygen that links the phosphate to the nucleoside), with nitrogen (bridged phosphoroamidates), sulfur (bridged phosphorothioates) and carbon (bridged methylenephosphonates). The replacement can occur at the either linking oxygen or at both the linking oxygens. When the bridging oxygen is the 3′-oxygen of a nucleoside, replacement with carbon is preferred. When the bridging oxygen is the 5′-oxygen of a nucleoside, replacement with nitrogen is preferred.

### Replacement of the Phosphate Group

The phosphate group can be replaced by non-phosphorus containing connectors. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include methyl phosphonate, hydroxylamino, siloxane, carbonate, carboxymethyl, carbamate, amide, thioether, ethylene oxide linker, sulfonate, sulfonamide, thioformacetal, formacetal, oxime, methylencimino, methylenemethylimino, methylenchydrazo, methylenedimethylhydrazo and methylencoxymethylimino. Preferred replacements include the methylenecarbonylamino and methylenemethylimino groups.

Modified phosphate linkages where at least one of oxygen linked to the phosphate has been replaced or the phosphate group has been replaced by a non-phosphorous group, are also referred to as “non phosphodiester backbone linkage.”

### Replacement of Ribophosphate Backbone

Oligonucleotide-mimicking scaffolds can also be constructed wherein the phosphate linker and ribose sugar are replaced by nuclease resistant nucleoside or nucleotide surrogates. While not wishing to be bound by theory, it is believed that the absence of a repetitively charged backbone diminishes binding to proteins that recognize polyanions (e.g., nucleases). Again, while not wishing to be bound by theory, it can be desirable in some embodiment, to introduce alterations in which the bases are tethered by a neutral surrogate backbone. Examples include the morpholino, cyclobutyl, pyrrolidine and peptide nucleic acid (PNA) nucleoside surrogates. Additional examples include morpholino which has nucleobases (e.g., adenine, cytosine, guanine and thyminc) linked to a morpholine ring, and phosphorodiamidate morpholino oligomer (PMO) which is a DNA analog that is built upon a backbone of morpholine rings connected by phosphorodiamidate linkages.

### Sugar Modifications

A modified RNA can include modification of all or some of the sugar groups of the ribonucleic acid, e.g., the 2′ hydroxyl group (OH) can be modified or replaced with a number of different “oxy” or “deoxy” substituents. While not being bound by theory, enhanced stability is expected since the hydroxyl can no longer be deprotonated to form a 2′-alkoxide ion. The 2′-alkoxide can catalyze degradation by intramolecular nucleophilic attack on the linker phosphorus atom. Again, while not wishing to be bound by theory, it can be desirable to some embodiments to introduce alterations in which alkoxide formation at the 2′position is not possible.

Examples of “oxy”-2′ hydroxyl group modifications include alkoxy or aryloxy (OR, e.g., R=H, alkyl, cycloalkyl, aryl, aralkyl, heteroaryl or sugar); polyethyleneglycol s (PEG), O(CH₂CH₂O) nCH₂CH₂OR; “locked” nucleic acids (LNA) in which the 2′ hydroxyl is connected, e.g., by a methylene bridge, to the 4′carbon of the same ribose sugar; O-AMINE (AMINE=NH₂; alkylamino, dialkylamino, heterocyclyl, arylamino, diaryl amino, heteroaryl amino, or diheteroaryl amino, ethylene diamine, polyamino) and aminoalkoxy, O(CH₂)ₙ AMINE, (e.g., AMINE=NH₂; alkylamino, dialkylamino, heterocyclyl, arylamino, diaryl amino, heteroaryl amino, or diheteroaryl amino, ethylene diamine, polyamino). It is noteworthy that oligonucleotides containing only the methoxy ethyl group (MOE), (OCH₂CH₂OCH₃, a PEG derivative), or 2′-O-methoxyethyl, exhibit nuclease stabilities comparable to those modified with the robust phosphorothioate modification.

“Deoxy” modifications include hydrogen (i.e. deoxyribose sugars, which are of particular relevance to the overhang portions of partially ds RNA); halo (e.g., fluoro); amino (e.g., N3/4; alkylamino, dialkylamino, heterocyclyl, arylamino, diaryl amino, heteroaryl amino, diheteroaryl amino, or amino acid); NH(CH₂CH₂NH) nCH₂CH₂— AMINE (AMINE=NH₂; alkylamino, dialkylamino, heterocyclyl, arylamino, diaryl amino, heteroaryl amino, or diheteroaryl amino), —NHC(O)R (R=alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino functionality. Preferred substitutents are 2′-methoxyethyl, 2′-OCH₃, 2′-O-allyl, 2′-C-allyl, and 2′-fluoro.

The sugar group can also contain one or more carbons that possess the opposite stereochemical configuration than that of the corresponding carbon in ribose. Thus, an oligonucleotide can include nucleotides containing e.g., arabinose, as the sugar. The monomer can have an alpha linkage at the position on the sugar, e.g., alpha-nucleosides. Oligonucleotides can also include “abasic” sugars, which lack a nucleobase at C—. These abasic sugars can also be further containing modifications at one or more of the constituent sugar atoms. Oligonucleotides can also contain one or more sugars that are in the L form, e.g., L-nucleosides.

### Terminal Modifications

The 3′ and 5′ ends of an oligonucleotide can be modified. Such modifications can be at the 3′ end, 5′ end or both ends of the molecule. They can include modification or replacement of an entire terminal phosphate or of one or more of the atoms of the phosphate group. E.g., the 3′ and 5′ends of an oligonucleotide can be conjugated to other functional molecular entities such as labeling moieties, e.g., fluorophores (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a linker. The terminal atom of the linker can connect to or replace the linking atom of the phosphate group or the C-3′ or C-5′O, N, S or C group of the sugar. Alternatively, the linker can connect to or replace the terminal atom of a nucleotide surrogate (e.g., PNAs). When a linker/phosphate-functional molecular entity-linker/phosphate array is interposed between two strands of a dsRNA, this array can substitute for a hairpin RNA loop in a hairpin-type RNA agent.

Terminal modifications useful for modulating activity include modification of the 5′end with phosphate or phosphate analogs. E.g., in preferred embodiments antisense strands of dsRNAs, are 5′phosphorylated or include a phosphoryl analog at the 5′prime terminus. 5′-phosphate modifications include those which are compatible with RISC mediated gene silencing. Suitable modifications include: 5′-monophosphate ((HO)₂(O) P—O-5′); 5′-diphosphate ((HO)₂ (O) P—O—P(HO) (O)—O-5′); 5′-triphosphate ((HO)₂ (O) P—O—(HO) (O) P—O—P(HO) (O)—O-5′); 5′-guanosine cap (7-methylated or non-methylated) (7m-G-O-5′-(HO) (O) P—O—(HO) (O) P—O—P(HO) (O)—O-5′); 5′-adenosine cap (Appp), and any modified or unmodified nucleotide cap structure (N—O-5′-(HO) (O) P—O—(HO) (O) P—O—P(HO) (O)—O-5′); 5′-monothiophosphate (phosphorothioate; (HO)₂ (S) P—O-5′); 5′-monodithiophosphate (phosphorodithioate; (HO) (HS) (S) P—O-5′), 5′-phosphorothiolate ((HO)₂ (O) P—S-5′); any additional combination of oxygen/sulfur replaced monophosphate, diphosphate and triphosphates (e.g., 5′-alpha-thiotriphosphate, 5′-gamma-thiotriphosphate, etc.), 5′-phosphoramidates ((HO) 2 (O) P—NH-5′, (HO) (NH₂) (O) P—O-5′), 5′-alkylphosphonates (R=alkyl=methyl, ethyl, isopropyl, propyl, etc., e.g., RP(OH)(O)—O-5′-, (OH)₂ (O) P-5′-CH₂—), 5′-alkyletherphosphonates (R=alkylether=methoxymethyl(MeOCH₂—), ethoxymethyl, etc., e.g., RP(OH)(O)—O-5′-).

Terminal modifications can also be useful for monitoring distribution, and in such embodiments the preferred groups to be added include fluorophores, e.g., fluorescein or an Alexa dye, e.g., Alexa 488. Terminal modifications can also be useful for enhancing uptake, useful modifications for this include cholesterol. Terminal modifications can also be useful for cross-linking an RNA agent to another moiety; modifications useful for this include mitomycin C.

### Nucleobases

Adenine, guanine, cytosine and uracil are the most common bases found in RNA. These bases can be modified or replaced to provide RNA's having improved properties. E.g., nuclease resistant oligoribonucleotides can be prepared with these bases or with synthetic and natural nucleobases (e.g., inosine, thymine, xanthine, hypoxanthine, nubularine, isoguanisine, or tubercidine) and any one of the above modifications. Alternatively, substituted or modified analogs of any of the above bases, e.g., “unusual bases”, “modified bases”, “non-natural bases” and “universal bases” described herein, can be employed. Examples include without limitation 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl) uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosinej-deazaadenine, N6, N6-dimethyladenine, 2, 6-diaminopurine, 5-amino-allyl-uracil, N3-methyluracil, substituted 1, 2, 4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl) uracil, 3-methylcytosine, 5-methylcytosine, N4-acetyl cytosine, 2-thiocytosine, N6-methyladenine, N6-isopentyladenine, 2-methylthio-N6-isopentenyladenine, N-methylguanines, or O-alkylated bases. Further purines and pyrimidines include those disclosed in U.S. Pat. No. 3,687,808, those disclosed in the Concise Encyclopedia Of Polymer Science And Engineering, pages 858-859, roschwitz, J. I., ed. John Wiley &Sons, 1990, and those disclosed by Uwe Englisch, et al. Chemically Modified Oligonucleotides as Probes and Inhibitors. 1991 June. Angewandte Chemie, International Edition. 30:613-629. doi: 10.1002/anie. 199106133.

### Cationic Groups

Modifications to oligonucleotides can also include attachment of one or more cationic groups to the sugar, base, and/or the phosphorus atom of a phosphate or modified phosphate backbone moiety. A cationic group can be attached to any atom capable of substitution on a natural, unusual or universal base. A preferred position is one that does not interfere with hybridization, i.e., does not interfere with the hydrogen bonding interactions needed for base pairing. A cationic group can be attached e.g., through the C2′position of a sugar or analogous position in a cyclic or acyclic sugar surrogate.

Cationic groups can include e.g., protonated amino groups, derived from e.g., O-AMINE (AMINE=N3/4; alkylamino, dialkylamino, heterocyclyl, arylamino, diaryl amino, heteroaryl amino, or diheteroaryl amino, ethylene diamine, polyamino); aminoalkoxy, e.g., 0 (CH₂) nAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g., NH₂; alkylamino, dialkylamino, heterocyclyl, arylamino, diaryl amino, heteroaryl amino, diheteroaryl amino, or amino acid); or NH(CH₂CH₂NH) nCH₂CH₂-AMINE (AMINE=NH₂; alkylamino, dialkylamino, heterocyclyl, arylamino, diaryl amino, heteroaryl amino, or diheteroaryl amino).

Placement within an Oligonucleotide

Some modifications may preferably be included on an oligonucleotide at a particular location, e.g., at an internal position of a strand, or on the 5′ or 3′ end of an oligonucleotide. A preferred location of a modification on an oligonucleotide, may confer preferred properties on the agent. For example, preferred locations of particular modifications may confer optimum gene silencing properties, or increased resistance to endonuclease or exonuclease activity.

One or more nucleotides of an oligonucleotide may have a 2′-5′ linkage. One or more nucleotides of an oligonucleotide may have inverted linkages, e.g., 3′-3′, 5′-5′, 2′-2′ or 2′-3′ linkages.

A double-stranded oligonucleotide may include at least one 5′-uridine-adenine-3′ (5′-UA-3′) dinucleotide wherein the uridine is a 2′-modified nucleotide, or a terminal 5′-uridine-guanine-3′ (5′-UG-3′) dinucleotide, wherein the 5′-uridine is a 2′-modified nucleotide, or a terminal 5′-cytidine-adenine-3′ (5′-CA-3′) dinucleotide, wherein the 5′-cytidine is a 2′-modified nucleotide, or a terminal 5′-uridine-uridine-3′ (5′-UU-3′) dinucleotide, wherein the 5′-uridine is a 2′-modified nucleotide, or a terminal 5′-cytidine-cytidine-3′ (5′-CC-3′) dinucleotide, wherein the 5′-cytidine is a 2′-modified nucleotide, or a terminal 5′-cytidine-uridine-3′ (5′-CU-3′) dinucleotide, wherein the 5′-cytidine is a 2′-modified nucleotide, or a terminal 5′-uridine-cytidine-3′ (5′-UC-3′) dinucleotide, wherein the 5′-uridine is a 2′-modified nucleotide. Double-stranded oligonucleotides including these modifications are particularly stabilized against endonuclease activity,

### General References

The oligoribonucleotides and oligoribonucleosides used in accordance with the present technology may be synthesized with solid phase synthesis, see for example “Oligonucleotide synthesis, a practical approach”, Ed. M. J. Gait, IRL Press, 1984; “Oligonucleotides and Analogues, A Practical Approach”, Ed. F. Eckstein, IRL Press, 1991 (especially Chapter 1, Modern machine-aided methods of oligodeoxyribonucl cotide synthesis, Chapter 2, Oligoribonucleotide synthesis, Chapter 3, 2′-0˜Methyloligoribonucleotide-s: synthesis and applications, Chapter 4, Phosphorothioate oligonucleotides, Chapter 5, Synthesis of oligonucleotide phosphorodithioates, Chapter 6, Synthesis of oligo-2′-deoxyribonucleoside methylphosphonates, and. Chapter 7, Oligodeoxynucleotides containing modified bases. Other particularly useful synthetic procedures, reagents, blocking groups and reaction conditions are described in P. Martin. Ein neuer Zugang zu 2′-O-Alkylribonucleosiden und Eigenschaften deren Oligonucleotide. 1995 Mar. 22. Helv. Chim. Acta. 78:486-504. doi: 10.1002/hlca. 19950780219; S. L. Beaucage and R. P. Iyer. Advances in the Synthesis of Oligonucleotides by the Phosphoramidite Approach. 1992 Mar. 20. Tetrahedron. 48:2223-2311. doi: 10.1016/S0040-4020 (01) 88752-4; S. L. Beaucage and R. P. Iyer. The synthesis of modified oligonucleotides by the phosphoramidite approach and their applications. 1993 Jul. 9. Tetrahedron. 49:6123-6194. doi: 10.1016/S0040-4020 (01) 87958-8, or references referred to therein. Modification described in WO 00/44895, WOO1/75164, or WO02/44321 can be used herein. The disclosure of all publications, patents, and published patent applications listed herein are hereby incorporated by reference.

The preparation of phosphinate oligoribonucleotides is described in U.S. Pat. No. 5,508,270. The preparation of alkyl phosphonate oligoribonucleotides is described in U.S. Pat. No. 4,469,863. The preparation of phosphoramidite oligoribonucleotides is described in U.S. Pat. No. 5,256,775 or U.S. Pat. No. 5,366,878. The preparation of phosphotriester oligoribonucleotides is described in U.S. Pat. No. 5,023,243. The preparation of borano phosphate oligoribonucleotide is described in U.S. Pat. Nos. 5,130,302 and 5,177,198. The preparation of 3′-Deoxy-3′-amino phosphoramidate oligoribonucleotides is described in U.S. Pat. No. 5,476,925. 3′-Deoxy-3′-methylenepho sphonate oligoribonucleotides is described in Haoyun An, et al. Synthesis of novel 3′-C-methylene thymidine and 5-methyluridinc/cytidine H-phosphonates and phosphonamidites for new backbone modification of oligonucleotides. 2001 Mar. 16. The Journal of Organic Chemistry. 66 (8), pp. 2789-2801. doi: 10.1021/jo001699u. Preparation of sulfur bridged nucleotides is described in Brian S. Sproat, et al. Synthesis of Modified Building Blocks Containing Amino or Thiol Moicties: Application of Modified Oligodeoxyribonucleotides. 2006 Dec. 6. Nucleosides Nucleotides. 7:651-653. doi: 10.1080/07328318808056302 and Crosstick et al. Tetrahedron Lett. 1989, 30, 4693.

Modifications to the 2′ sugar group can be found in S. Verma, et al. MODIFIED OLIGONUCLEOTIDES: Synthesis and Strategy for Users. 1998 July. Annu. Rev. Biochem. 67:99-134. doi: 10.1146/annurev. biochem. 67.1.99 and all references therein. Specific modifications to the ribose can be found in the following references: 2′-fluoro (Kawasaki, et. al. Uniformly modified 2′-deoxy-2′-fluoro-phosphorothioate oligonucleotides as nuclease-resistant antisense compounds with high affinity and specificity for RNA targets. 1993 Apr. 1. J. Med. Chem. 36:831-841. doi: 10.1021/jm00059a007), 2′-MOE (P. Martin. Stereoselektive Synthese von 2′-O-(2-Methoxyethyl) ribonucleosiden: Nachbargruppenbeteiligung der Methoxyethoxy-Gruppe bei der Ribosylierung von Heterocyclen. 1996 Oct. 30. Helv. Chim. Acta. 79:1930-1938. doi: 10.1002/hlca. 19960790716), “LNA” (J. Wengel. Synthesis of 3′-C- and 4′-C-Branched Oligodeoxynucleotides and the Development of Locked Nucleic Acid (LNA). 1998 Dec. 4. Acc. Chem. Res. 32:301-310. doi: 10.1021/ar980051p).

Methylenemethylimino linked oligoribonucleosides, also identified herein as MMI linked oligoribonucleosides, methylenedimethylhydrazo linked oligoribonucleosides, also identified herein as MDH linked oligoribonucleosides, and methylenecarbonylamino linked oligonucleosides, also identified herein as amide-3 linked oligoribonucleosides, and methylencaminocarbonyl linked oligonucleosides, also identified herein as amide-4 linked oligoribonucleosides as well as mixed backbone compounds having, as for instance, alternating MMI and PO or PS linkages can be prepared as is described in U.S. Pat. Nos. 5,378,825, 5,386,023, 5,489,677 and in published PCT applications PCT/US92/04294 and PCT/US92/04305 (published as WO 92/20822 WO and 92/20823, respectively). Formacetal and thioformacetal linked oligoribonucleosides can be prepared as is described in U.S. Pat. Nos. 5,264,562 and 5,264,564. Ethylene oxide linked oligoribonucleosides can be prepared as is described in U.S. Pat. No. 5,223,618. Siloxane replacements are described in James F. Cormier, et al. Synthesis of hexanucleotide analogues containing diisopropylsilyl internucleotide linkages. 1988 May 25. Nucleic Acids Res. 16:4583-4594. doi: 10.1093/nar/16.10.4583. Carbonate replacements are described in J. R. Tittensor. The preparation of nucleoside carbonates. 1971 January. Chem. Soc. C. 2656-2662. doi: 10.1039/J39710002656. Carboxymethyl replacements are described in M. D. Edge, et al. Synthetic analogues of polynucleotides. Part VIII. Analogues of oligonucleotides containing carboxymethylthymidinc. 1991. J. Chem. Soc. Perkin Trans. 1.1972. doi: 10.1039/P19720001991. Carbamate replacements are described in E. P. Stirchak, et al. Uncharged stercoregular nucleic acid analogs: 2. Morpholino nucleoside oligomers with carbamate internucleoside linkages. 1989 Aug. 11. Nucleic Acids Res. 17, 6129. doi: 10.1093/nar/17.15.6129.

Cyclobutyl sugar surrogate compounds can be prepared as is described in U.S. Pat. No. 5,359,044. Pyrrolidine sugar surrogate can be prepared as is described in U.S. Pat. No. 5,519,134. Morpholino sugar surrogates can be prepared as is described in U.S. Pat. Nos. 5,142,047 and 5,235,033, and other related patent disclosures. Peptide Nucleic Acids (PNAs) are known per se and can be prepared in accordance with any of the various procedures referred to in Peptide Nucleic Acids (PNA): Synthesis, Properties and Potential Applications, Bioorganic &Medicinal Chemistry, 1996, 4, 5-23. They may also be prepared in accordance with U.S. Pat. No. 5,539,083.

Terminal modifications are described in M. Manoharan, et al. Oligonucleotide Conjugates as Potential Antisense Drugs with Improved Uptake, Biodistribution, Targeted Delivery, and Mechanism of Action. 2004 Jul. 8. Antisense and Nucleic Acid Drug Development. 12, 103-128. doi: 10.1089/108729002760070849 and references therein. Nucleobases References N-2 substituted purine nucleoside amidites can be prepared as is described in U.S. Pat. No. 5,459,255. 3-Deaza purine nucleoside amidites can be prepared as is described in U.S. Pat. No. 5,457,191. 5, 6-Substituted pyrimidine nucleoside amidites can be prepared as is described in U.S. Pat. No. 5,614,617. 5-Propynyl pyrimidine nucleoside amidites can be prepared as is described in U.S. Pat. No. 5,484,908.

### Manufacture of ASOs

The ASOs (e.g., bipartite ASOs) used in accordance with the present technology may be conveniently and routinely made through the well-known technique of solid phase synthesis. Equipment for such synthesis is sold by several vendors including, for example, Applied Biosystems (Foster City, Calif.). One method for synthesizing oligonucleotides on a modified solid support is described in U.S. Pat. No. 4,458,066.

Any other means for such synthesis known in the art may additionally or alternatively be employed. It is well known to use similar techniques to prepare oligonucleotides such as the phosphorothioates and alkylated derivatives. In one such automated embodiment, diethyl-phosphoramidites are used as starting materials and may be synthesized as described by S. L. Beaucage, et al. Deoxynucleoside phosphoramidites-A new class of key intermediates for deoxypolynucleotide synthesis. 2001 Mar. 9. Tetrahedron Letters. 22:1859-1862. doi: 10.1016/S0040-4039 (01) 90461-7.

The ASOs of the present technology may be synthesized in vitro and do not include ASOs of biological origin, or genetic vector constructs designed to direct the in vivo synthesis of ASOs. The molecules of the present technology may also be mixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption.

Methods of Improving Exon Skipping Efficacy and/or Efficiency of an ASO Comprising or Consisting of a Targeting Sequence

In some aspects, the present technology provides a method of improving exon skipping efficacy and/or efficiency of an ASO comprising or consisting of a targeting sequence comprising providing one or more bipartite ASOs comprising the targeting sequence and a 5′-splice-site decoy sequence disclosed herein. In certain embodiments, each decoy sequence of the one or more bipartite ASOs comprises or consists of a nucleotide sequence selected from the group consisting of the nucleotide sequences of SEQ ID Nos. 1 to 23 and 347 to 353. In certain embodiments, at least some of the one or more bipartite ASOs has the decoy sequence operably connected at the 5′ end of the targeting sequence. In certain embodiments, at least some of the one or more bipartite ASOs has the decoy sequence operably connected at the 3′ end of the targeting sequence. In certain embodiments, at least some of the one or more bipartite ASOs has the decoy sequence operably connected at both the 5′ and 3′ ends of the targeting sequence. In certain embodiments, at least some of the one or more bipartite ASOs has the decoy sequence operably connected at only one of the 5′ and 3′ ends of the targeting sequence.

In certain embodiments, the method further comprises screening and/or optimizing one or more bipartite ASOs according to their exon skipping efficacies and/or efficiencies. In certain embodiments, the bipartite ASOs increase the exon skipping efficiency of the targeting sequence by about 2 fold or higher, about 3 fold or higher, about 4 fold or higher, about 5 fold or higher, about 6 fold or higher, about 7 fold or higher, about 8 fold or higher, about 9 fold or higher, about 10 fold or higher, about 11 fold or higher, about 12 fold or higher, about 13 fold or higher, about 14 fold or higher, about 15 fold or higher, about 16 fold or higher or higher, about 17 fold or higher, about 18 fold or higher, about 19 fold or higher, about 20 fold or higher, about 25 fold or higher, about 30 fold or higher, about 40 fold or higher, about 50 fold or higher, about 60 fold or higher, about 70 fold or higher, about 80 fold or higher, about 90 fold or higher, about 100 fold or higher, or more, including all values and ranges between these values, compared to an ASO having the identical targeting sequence but no decoy sequence.

In certain embodiments, the targeting sequence is capable of hybridizing to a sequence selected from the group consisting of an exon of interest, a flanking intron sequence upstream of the exon of interest, a flanking intron sequence downstream of the exon of interest, an intron-exon junction upstream of the exon of interest, and an intron-exon junction downstream of the exon of interest in a cell or subject.

### Compositions of Bipartite ASOs

In some aspects, the present technology provides compositions comprising a bipartite ASO disclosed herein and a pharmaceutically acceptable carrier. In certain embodiments, the composition is a pharmaceutical formulation or composition.

### Vector Encoding Bipartite ASOs

In some aspects, the present technology provides a vector encoding the ASO (e.g., bipartite ASO) disclosed herein.

### Use of Bipartite ASOs for Treatment of Diseases

In some aspects, the present technology provides a method of treating a disease and/or complications thereof in a subject comprising administering to the subject the ASO (e.g., bipartite ASO) disclosed herein, the composition (pharmaceutical composition) comprising the ASO (e.g., bipartite ASO) as disclosed herein, and/or the vector encoding the ASO (e.g., bipartite ASO) as disclosed herein. The ASO (e.g., bipartite ASO) may generate or promote exon skipping of the exon of interest during pre-mRNA splicing. The ASO-mediated exon skipping may be an approach to manipulate expression of a gene of interest. The expression of the gene of interest may be manipulated by ASOs to inhibit splicing of an exon, intron or a specific splice site of a gene of interest, leading to, e.g., without limitation, restoring the reading frame of a defective gene of interest, generating a different isoform of a gene of interest (e.g., a dominant negative isoform), skipping a toxic part of the gene, silencing the gene, and/or changing the structure and function of a gene.

In some aspects, the present technology provides a use of the ASO (e.g., bipartite ASO) disclosed herein, the composition (e.g., pharmaceutical composition) comprising the ASO (e.g., bipartite ASO) as disclosed herein, and/or the vector encoding the ASO (e.g., bipartite ASO) as disclosed herein to treat a disease and/or complications thereof. Examples of the disease include, without limitation, those disclosed herein.

In some aspects, the present technology provides the ASO (e.g., bipartite ASO) disclosed herein, the composition (e.g., pharmaceutical composition) comprising the ASO (e.g., bipartite ASO) as disclosed herein, and/or the vector encoding the ASO (e.g., bipartite ASO) as disclosed herein for use in treating a disease and/or complications thereof. Examples of the disease include, without limitation, those disclosed herein.

Examples of the diseases and/or complications thereof include, without limitation, the diseases and/or complications thereof that may benefit from exon skipping on one or more genes of interest. In certain embodiments, the gene of interest is selected from the group consisting of SMN1, SMN2, DMD, APP, CEP290, HER2, SCA3, PKM, and MDM4 genes.

Examples of the disease and/or complications thereof include, without limitation, those that can be treated by exon skipping of the exon of interest. In certain embodiments, the exon of interest is selected from the group consisting of exon 7 of the endogenous SMN1 and SMN2 genes, exons 45, 51 and 53 of the endogenous DMD gene, exon 17 of the endogenous APP gene, exon 41 of the endogenous CEP290 gene, exon 19 of the endogenous HER2 gene, exon 10 of the SCA3 gene, exon 10 of the endogenous PKM gene, and exon 6 of the endogenous MDM4 gene.

Examples of the disease and/or complications thereof include, without limitation, those that can be treated by inhibiting splicing of an exon, intron or a specific splice site of a gene of interest

Examples of the disease and/or complications thereof include, without limitation, those that can be treated by restoring the reading frame of a defective gene of interest.

Examples of the disease and/or complications thereof include, without limitation, those that can be treated by generating a different isoform of a gene of interest (e.g., a dominant negative isoform).

Examples of the disease and/or complications thereof include, without limitation, those that can be treated by skipping a toxic part of the gene.

Examples of the disease and/or complications thereof include, without limitation, those that can be treated by silencing the gene.

Examples of the disease and/or complications thereof include, without limitation, those that can be treated by changing the structure and function of a gene to obtain a beneficial or desirable isoform.

Examples of the disease and/or complications thereof include, without limitation, those that can be treated by production of a functional protein encoded by the different isoform of the gene of interest.

Examples of the disease and/or complications thereof may include, without limitation, Duchenne muscular dystrophy (DMD) Alzheimer's disease, Joubert Syndrome, spinocerebellar ataxia 3 (SCA3), cancer, e.g., without limitation, breast cancer, HER2-positive biliary tract, colorectal, non-small-cell lung, bladder cancers, prostate cancer, lung cancer, cervix cancer, kidney cancer, papillary thyroid cancer, colon cancer, colorectal cancer, gliomas, ovarian cancer, gastric cancer, hepatoblastoma, fibrolamellar, hepatocellular, carcinoma, soft tissue sarcoma, osteosarcoma, chronic lymphocytic, leukemia, acute myeloid leukemia, mantle cell lymphoma, Pediatric Burkitt, lymphoma, salivary gland cancer, liver cancer, and melanoma.

In certain embodiments, the ASO (e.g., bipartite ASO), the composition (e.g., pharmaceutical composition) comprising the ASO (e.g., bipartite ASO), and/or the vector encoding the ASO (e.g., bipartite ASO) are administered at a therapeutically effective amount.

Examples of the subject include, without limitation, mammals such as human.

In some embodiments, one or more ASOs designed using one or more methods of the present technology can be used for the treatment of diseases treatable with exon skipping. For example, spinocerebellar ataxia type 3 (SCA3) is a neurodegenerative disorder caused by a CAG triplet expansion in exon 10 of the ATXN3 gene. ASOs may be used to promote skipping of exon 9, exon 10, or both to favor potentially functional, partially functional, or non-toxic ATXN3 variants.

“Treatment” of an individual (e.g., a mammal, such as a human) or a cell is any type of intervention used in an attempt to alter the natural course of the individual or cell. Treatment includes, but is not limited to, administration of a pharmaceutical composition or combination therapy, and may be performed either prophylactically or subsequent to the initiation of a pathologic event or contact with an etiologic agent. Treatment includes any desirable effect on the symptoms or pathology of a disease or condition associated with the dystrophin protein, as in some forms of muscular dystrophy, and may include, for example, minimal changes or improvements in one or more measurable markers of the disease or condition being treated. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

In some embodiments, treatment with one or more ASOs of the present technology, or one or more ASOs of the present technology in combination with one or more additional therapeutic agents (combination therapy), induces or increases novel production of functional protein encoded by the gene of interest (e.g., dystrophin in DMD), delays disease progression, slows or reduces the symptoms of the disease (e.g., for DMD, the loss of ambulation, reduces muscle inflammation, reduces muscle damage, improves muscle function, reduces loss of pulmonary function, and/or enhances muscle regeneration), or any combination thereof, that would be expected without treatment. In some embodiments, treatment maintains, delays, or slows disease progression.

In some embodiments when the disease is DMD, the treatment maintains ambulation or reduces the loss of ambulation. In some embodiments, treatment maintains pulmonary function or reduces loss of pulmonary function. In some embodiments, treatment maintains or increases a stable walking distance in a patient, as measured by, for example, the 6 Minute Walk Test (6MWT). In some embodiments, treatment maintains, improves, or reduces the time to walk/run 10 meters (i.e., the 10 meter walk/run test). In some embodiments, treatment maintains, improves, or reduces the time to stand from supine (i.e., time to stand test). In some embodiments, treatment maintains, improves, or reduces the time to climb four standard stairs (i.e., the four-stair climb test). In some embodiments, treatment maintains, improves, or reduces muscle inflammation in the patient, as measured by, for example, MRI (e.g., MRI of the leg muscles). In some embodiments, MRI measures a change in the lower leg muscles. In some embodiments, MRI measures T2 and/or fat fraction to identify muscle degeneration. MRI can identify changes in muscle structure and composition caused by inflammation, edema, muscle damage and fat infiltration. In some embodiments, muscle strength is measured by the North Star Ambulatory Assessment. In some embodiments, muscle strength is measured by the pediatric outcomes data collection instrument (PODCI).

In some embodiments, treatment with one or more ASOs of the present technology, or one or more ASOs of the present technology in combination with one or more additional therapeutic agents (combination therapy).

In some embodiments when the disease is DMD, such treatment reduces muscle inflammation, reduces muscle damage, improves muscle function, and/or enhances muscle regeneration. For example, treatment may stabilize, maintain, improve, or reduce inflammation in the subject. Treatment may also, for example, stabilize, maintain, improve, or reduce muscle damage in the subject. Treatment may, for example, stabilize, maintain, or improve muscle function in the subject. In addition, for example, treatment may stabilize, maintain, improve, or enhance muscle regeneration in the subject. In some embodiments, treatment maintains, improves, or reduces muscle inflammation in the patient, as measured by, for example, magnetic resonance imaging (MRI) (e.g., MRI of the leg muscles) that would be expected without treatment. In some embodiments, treatment with one or more ASOs of the present technology, or one or more ASOs of the present technology in combination with one or more additional therapeutic agents (combination therapy), increases novel dystrophin production and slows or reduces the loss of ambulation that would be expected without treatment. For example, treatment may stabilize, maintain, improve or increase walking ability (e.g., stabilization of ambulation) in the subject. In some embodiments, treatment maintains or increases a stable walking distance in a patient, as measured by, for example, the 6 Minute Walk Test (6MWT), described by Craig M. McDonald, et al. The 6-minute walk test in Duchenne/Becker muscular dystrophy: Longitudinal observations. 2010 Oct. 29. Muscle Nerve. 42:966-74. doi: 10.1002/mus. 21808, herein incorporated by reference). A change in the 6 Minute Walk Distance (6MWD) may be expressed as an absolute value, a percentage change or a change in the %-predicted value. In some embodiments, treatment maintains or improves a stable walking distance in a 6MWT from a 20% deficit in the subject relative to a healthy peer. The performance of a DMD patient in the 6MWT relative to the typical performance of a healthy peer can be determined by calculating a %-predicted value. For example, the %-predicted 6MWD may be calculated using the following equation for males: 196.72+(39.81×age)−(1.36×age2)+(132.28×height in meters). For females, the %-predicted 6MWD may be calculated using the following equation: 188.61+(51.50×age)−(1.86×age2)+(86.10×height in meters) (Henricson et al. PLoS Curr., 2012, version 2, herein incorporated by reference). In some embodiments, treatment increases the stable walking distance in the patient from baseline to greater than 3, 5, 6, 7, 8, 9, 10, 15, 20, 25, 30 or 50 meters (including all integers in between). Loss of muscle function in patients with DMD may occur against the background of normal childhood growth and development. Indeed, younger children with DMD may show an increase in distance walked during 6MWT over the course of about 1 year despite progressive muscular impairment. In some embodiments, the 6MWD from patients with DMD is compared to typically developing control subjects and to existing normative data from age and sex matched subjects. In some embodiments, normal growth and development can be accounted for using an age and height based equation fitted to normative data. Such an equation can be used to convert 6MWD to a percent-predicted (%-predicted) value in subjects with DMD. In some embodiments, analysis of %-predicted 6MWD data represents a method to account for normal growth and development, and may show that gains in function at early ages (e.g., less than or equal to age 7) represent stable rather than improving abilities in patients with DMD (Henricson et al. PLOS Curr., 2012, version 2, herein incorporated by reference).

### Pharmaceutical Formulation (Composition) and Delivery

In some embodiments, the present technology provides formulations or compositions suitable for the therapeutic delivery of ASOs, as described herein. Hence, in some embodiments, the present technology provides pharmaceutically acceptable compositions that comprise a therapeutically-effective amount of one or more of the oligomers described herein, formulated together with one or more pharmaceutically acceptable carriers (additives) and/or diluents. While it is possible for an oligomer of the present technology to be administered alone, it is preferable to administer the compound as a pharmaceutical formulation (composition).

Methods for the delivery of nucleic acid molecules are described, for example, in Akhtar, et al. Cellular uptake and intracellular fate of antisense oligonucleotides. 1992 May. Trends in Cell Bio. 2:139-144. doi: 10.1016/0962-8924 (92) 90100-2; and Delivery Strategies for ASO Therapeutics, ed. Akhtar; Sullivan et al., PCT WO 94/02595. These and other protocols can be utilized for the delivery of virtually any nucleic acid molecule, including the isolated oligomers of the present technology.

As detailed below, the pharmaceutical compositions of the present technology may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

Some examples of materials that can serve as pharmaceutically acceptable carriers include, without limitation: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

Additional non-limiting examples of agents suitable for formulation with the ASOs of the present technology include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues; biodegradable polymers, such as poly (DL-lactide-coglycolide) microspheres for sustained release delivery after implantation (D. F. Emerich, et al. Biocompatibility of Poly (DL-Lactide-co-Glyc olide) Microspheres Implanted into the Brain. 1999 January. Cell Transplant. 8, 47-58. doi: 10.1177/096368979900800114) Alkermes, Inc. Cambridge, Mass.; and loaded nanoparticles, such as those made of polybutylcyanoacrylate, which can deliver drugs across the blood brain barrier and can alter neuronal uptake mechanisms (U. Schroeder, et al. Diffusion enhancement of drugs by loaded nanoparticles in vitro. 1999 Jul. 1. Prog Neuropsychopharmacol Biol Psychiatry. 23, 941-949. doi: 10.1016/s0278-5846 (99) 00037-8).

The present technology also features the use of the composition comprising surface-modified liposomes containing poly (ethylene glycol) lipids (PEG-modified, branched and unbranched or combinations thereof, or long-circulating liposomes or stealth liposomes). Oligomers of the present technology can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Danilo D. Lasic, et al. The “Stealth” Liposome: A Prototypical Biomaterial. 1995 Dec. 1. Chem. Rev. 95, 2601-2627. doi: 10.1021/cr00040a001; H. Ishiwata, et al. Physical-Chemistry Characteristics and Biodistribution of Poly (ethylene glycol)-Coated Liposomes Using Poly (oxyethylene) Cholesteryl Ether. 1995 Junc. Chem. Pharm. Bull. 43, 1005-1011. doi: 10.1248/cpb. 43.1005). Such liposomes have been shown to accumulate selectively in tumors, presumably by extravasation and capture in the neovascularized target tissues (Danilo D. Lasic, et al. Liposomes Revisited. 1995 Mar. 3. Science. 267, 1275-1276. doi: 10.1126/science. 7871422; Naoto Oku, et al. Real-time analysis of liposomal trafficking in tumor-bearing mice by use of positron emission tomography. 1995 Aug. 23. Biochimica et Biophysica Acta (BBA)-Biomembranes. 1238, 86-90. doi: 10.1016/0005-2736 (95) 00106-D). The long-circulating liposomes enhance the pharmacokinetics and pharmacodynamics of DNA and RNA, particularly compared to conventional cationic liposomes which are known to accumulate in tissues of the MPS (Y. Liu, et al. Cationic liposome-mediated intravenous gene delivery. 1995 Oct. 20. J. Biol. Chem. 42, 24864-24870. doi: 10.1074/jbc. 270.42.24864; Choi et al., International PCT Publication No. WO 96/10391; Ansell et al., International PCT Publication No. WO 96/10390; Holland et al., International PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

In some embodiments, the present technology includes oligomer compositions prepared for delivery as described in U.S. Pat. Nos. 6,692,911, 7,163,695 and 7,070,807. In this regard, in one embodiment, the present technology provides an oligomer of the present technology in a composition comprising copolymers of lysine and histidine (HK) (as described in U.S. Pat. Nos. 7,163,695, 7,070,807, and 6,692,911) cither alone or in combination with PEG (e.g., branched or unbranched PEG or a mixture of both), in combination with PEG and a targeting moiety or any of the foregoing in combination with a crosslinking agent. In some embodiments, the present technology provides ASOs in compositions comprising gluconic-acid-modified polyhistidine or gluconylated-polyhistidine/transferrin-polylysine. One skilled in the art will also recognize that amino acids with properties similar to His and Lys may be substituted within the composition.

Some embodiments of the oligomers described herein may contain a basic functional group, such as amino or alkylamino, and are, thus, capable of forming pharmaceutically acceptable salts with pharmaceutically acceptable acids. These salts can be prepared in situ in the administration vehicle or the dosage form manufacturing process, or by separately reacting a purified compound of the present technology in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (Sec, e.g., S. M. Berge, et al. Pharmaceutical Salts. 1977 January. J. Pharm. Sci. 66:1-19. doi: 10.1002/jps. 2600660104).

The pharmaceutically acceptable salts of the subject oligomers include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In some embodiments, the oligomers of the present technology may contain one or more acidic functional groups and, thus, are capable of forming pharmaceutically acceptable salts with pharmaceutically acceptable bases. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, dicthanolamine, piperazine and the like. (See, e.g., S. M. Berge, et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present technology include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient that can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, or from about 5 percent to about 70 percent, or from about 10 percent to about 30 percent.

In some embodiments, a formulation of the present technology comprises an excipient selected from cyclodextrins, celluloses, liposomes, micelle forming agents, e.g., bile acids, and polymeric carriers, e.g., polyesters and polyanhydrides; and an oligomer of the present technology. In some embodiments, an aforementioned formulation renders orally bioavailable an oligomer of the present technology.

Methods of preparing these formulations or compositions include the step of bringing into association an oligomer of the present technology with the carrier and, optionally, one or more accessory ingredients. In general, the formulations are prepared by uniformly and intimately bringing into association a compound of the present technology with liquid carriers, or finely divided solid carriers, or both, and then, if necessary, shaping the product.

Formulations of the present technology suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present technology as an active ingredient. An oligomer of the present technology may also be administered as a bolus, electuary or paste.

In solid dosage forms of the present technology for oral administration (capsules, tablets, pills, dragees, powders, granules, trouches and the like), the active ingredient may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, some silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (e.g., gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present technology, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the present technology include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more compounds of the present technology with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations or dosage forms for the topical or transdermal administration of an oligomer as provided herein include powders, sprays, ointments, pastes, creams, lotions, gels, solutions, patches and inhalants. The active oligomers may be mixed under sterile conditions with a pharmaceutically acceptable carrier, and with any preservatives, buffers, or propellants which may be required. The ointments, pastes, creams and gels may contain, in addition to an active compound of the present technology, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an oligomer of the present technology, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of an oligomer of the present technology to the body. Such dosage forms can be made by dissolving or dispersing the oligomer in the proper medium. Absorption enhancers can also be used to increase the flux of the agent across the skin. The rate of such flux can be controlled by either providing a rate controlling membrane or dispersing the agent in a polymer matrix or gel, among other methods known in the art.

Pharmaceutical compositions suitable for parenteral administration may comprise one or more oligomers of the present technology in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the present technology include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject oligomers may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some embodiments, in order to prolong the effect of a drug, it is desirable to slow the absorption of the drug from subcutaneous or intramuscular injection. This may be accomplished by the use of a liquid suspension of crystalline or amorphous material having poor water solubility, among other methods known in the art. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms may be made by forming microencapsule matrices of the subject oligomers in biodegradable polymers such as polylactide-polyglycolide. Depending on the ratio of oligomer to polymer, and the nature of the particular polymer employed, the rate of oligomer release can be controlled. Examples of other biodegradable polymers include poly(orthoesters) and poly(anhydrides). Depot injectable formulations may also be prepared by entrapping the drug in liposomes or microemulsions that are compatible with body tissues.

When the ASOs of the present technology are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

As noted above, the formulations or preparations of the present technology may be given orally, parenterally, topically, or rectally. They are typically given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

Regardless of the route of administration selected, the ASOs of the present technology, which may be used in a suitable hydrated form, and/or the pharmaceutical compositions of the present technology, may be formulated into pharmaceutically acceptable dosage forms by conventional methods known to those of skill in the art. Actual dosage levels of the active ingredients in the pharmaceutical compositions of the present technology may be varied so as to obtain an amount of the active ingredient which is effective to achieve the desired therapeutic response for a particular patient, composition, and mode of administration, without being unacceptably toxic to the patient.

The selected dosage level will depend upon a variety of factors including the activity of the particular oligomer of the present technology employed, or the ester, salt or amide thereof, the route of administration, the time of administration, the rate of excretion or metabolism of the particular oligomer being employed, the rate and extent of absorption, the duration of the treatment, other drugs, compounds and/or materials used in combination with the particular oligomer employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the present technology employed in the pharmaceutical composition at levels lower than that required in order to achieve the desired therapeutic effect and gradually increase the dosage until the desired effect is achieved. In general, a suitable daily dose of a compound of the present technology will be that amount of the compound which is the lowest dose effective to produce a therapeutic effect. Such an effective dose will generally depend upon the factors described above. Generally, oral, intravenous, intracerebroventricular and subcutaneous doses of the compounds of the present technology for a patient, when used for the indicated effects, will range from about 0.001 to about 1,000 mcg/g/day, about 0.01 to about 500 mcg/g/day, about 0.1 to about 200 mcg/g/day, about 1 to about 160 mcg/g/day, or about 10 to about 150 mcg/g/day. If desired, the effective daily dose of the active compound may be administered as two, three, four, five, six or more sub-doses administered separately at appropriate intervals throughout the day, optionally, in unit dosage forms. In some situations, dosing is one administration per day. In some embodiments, dosing is one or more administration per every 2, 3, 4, 5, 6, 7, 8, 9, 10, 11, 12, 13, 14 days, or every 1, 2, 3, 4, 5, 6, 7, 8, 9, 10, 11, 12 weeks, or every 1, 2, 3, 4, 5, 6, 7, 8, 9, 10, 11, 12 months, as needed, to maintain the desired expression of a functional dystrophin protein.

Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres, as described herein and known in the art. In some embodiments, microemulsification technology may be utilized to improve bioavailability of lipophilic (water insoluble) pharmaceutical agents. Examples include Trimetrine (S. K. Dordunoo, et al. Preformulation Studies on Solid Dispersions Containing Triamterene or Temazepam in Polyethylene Glycols or Gelucire 44/14 for Liquid Filling of Hard Gelatin Capsules. 2008 October. Drug Development and Industrial Pharmacy. 17 (12), 1685-1713. doi: 10.3109/03639049109057315 and REV 5901 (P. C. Sheen, et al. Bioavailability of a Poorly Water-Soluble Drug from Tablet and Solid Dispersion in Humans. 1991 July. J. Pharm. Sci. 80 (7), 712-714. doi: 10.1002/jps. 2600800722). Among other benefits, microemulsification provides enhanced bioavailability by preferentially directing absorption to the lymphatic system instead of the circulatory system, which thereby bypasses the liver, and prevents destruction of the compounds in the hepatobiliary circulation.

In some embodiments, the formulations contain micelles formed from an oligomer as provided herein and at least one amphiphilic carrier, in which the micelles have an average diameter of less than about 100 nm. More preferred embodiments provide micelles having an average diameter less than about 50 nm, and even more preferred embodiments provide micelles having an average diameter less than about 30 nm, or even less than about 20 nm.

While all suitable amphiphilic carriers are contemplated, the presently preferred carriers are generally those that have Generally-Recognized-as-Safe (GRAS) status, and that can both solubilize the compound of the present technology and microemulsify it at a later stage when the solution comes into a contact with a complex water phase (such as one found in human gastrointestinal tract). Usually, amphiphilic ingredients that satisfy these requirements have HLB (hydrophilic to lipophilic balance) values of 2-20, and their structures contain straight chain aliphatic radicals in the range of C-6 to C-20. Examples are polyethylene-glycolized fatty glycerides and polyethylene glycols.

Examples of amphiphilic carriers include saturated and monounsaturated polyethyleneglycolyzed fatty acid glycerides, such as those obtained from fully or partially hydrogenated various vegetable oils. Such oils may advantageously consist of tri-, di-, and mono-fatty acid glycerides and di- and mono-polyethyleneglycol esters of the corresponding fatty acids, with a particularly preferred fatty acid composition including capric acid 4-10, capric acid 3-9, lauric acid 40-50, myristic acid 14-24, palmitic acid 4-14 and stearic acid 5-15%. Another useful class of amphiphilic carriers includes partially esterified sorbitan and/or sorbitol, with saturated or mono-unsaturated fatty acids (SPAN-series) or corresponding ethoxylated analogs (TWEEN-series).

Commercially available amphiphilic carriers may be particularly useful, including Gelucire-series, Labrafil, Labrasol, or Lauroglycol (all manufactured and distributed by Gattefosse Corporation, Saint Priest, France), PEG-mono-oleate, PEG-di-oleate, PEG-mono-laurate and di-laurate, Lecithin, Polysorbate 80, etc. (produced and distributed by a number of companies in USA and worldwide).

In some embodiments, the delivery may occur by use of liposomes, nanocapsules, microparticles, microspheres, lipid particles, vesicles, and the like, for the introduction of the compositions of the present technology into suitable host cells. In particular, the compositions of the present technology may be formulated for delivery either encapsulated in a lipid particle, a liposome, a vesicle, a nanosphere, a nanoparticle or the like. The formulation and use of such delivery vehicles can be carried out using known and conventional techniques.

Hydrophilic polymers suitable for use in the present technology are those which are readily water-soluble, can be covalently attached to a vesicle-forming lipid, and which are tolerated in vivo without toxic effects (i.e., are biocompatible). Suitable polymers include polyethylene glycol (PEG), polylactic (also termed polylactide), polyglycolic acid (also termed polyglycolide), a polylactic-polyglycolic acid copolymer, and polyvinyl alcohol. In some embodiments, polymers have a molecular weight of from about 100 or 120 daltons up to about 5,000 or 10,000 daltons, or from about 300 daltons to about 5,000 daltons. In other embodiments, the polymer is polyethyleneglycol having a molecular weight of from about 100 to about 5,000 daltons, or having a molecular weight of from about 300 to about 5,000 daltons. In some embodiments, the polymer is polyethyleneglycol of 750 daltons (PEG (750)). Polymers may also be defined by the number of monomers therein; a preferred embodiment of the present technology utilizes polymers of at least about three monomers, such PEG polymers consisting of three monomers (approximately 150 daltons).

Other hydrophilic polymers which may be suitable for use in the present technology include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

In some embodiments, a formulation of the present technology comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly (ortho) esters, poly (butic acid), poly(valeric acid), poly(lactide-co-caprolactone), polysaccharides, proteins, polyhyaluronic acids, polycyanoacrylates, and blends, mixtures, or copolymers thereof.

Cyclodextrins are cyclic oligosaccharides, consisting of 6, 7 or 8 glucose units, designated by the Greek letter α, β, or γ, respectively. The glucose units are linked by α-1,4-glucosidic bonds. As a consequence of the chair conformation of the sugar units, all secondary hydroxyl groups (at C-2, C-3) are located on one side of the ring, while all the primary hydroxyl groups at C-6 are situated on the other side. As a result, the external faces are hydrophilic, making the cyclodextrins water-soluble. In contrast, the cavities of the cyclodextrins are hydrophobic, since they are lined by the hydrogen of atoms C-3 and C-5, and by ether-like oxygens. These matrices allow complexation with a variety of relatively hydrophobic compounds, including, for instance, steroid compounds such as 17α-estradiol (see, e.g., Wim van Uden, et al. Cyclodextrins as a useful tool for bioconversions in plant cell biotechnology. 1994 September. Plant Cell Tissue and Org. Cult. 38:103-113. doi: 10.1007/BF00033867). The complexation takes place by Van der Waals interactions and by hydrogen bond formation. For a general review of the chemistry of cyclodextrins, see, Wenz, Agnew. Chem. Int. Ed. Engl., 33:803-822 (1994).

The physico-chemical properties of the cyclodextrin derivatives depend strongly on the kind and the degree of substitution. For example, their solubility in water ranges from insoluble (e.g., triacetyl-beta-cyclodextrin) to 147% soluble (w/v) (G-2-beta-cyclodextrin). In addition, they are soluble in many organic solvents. The properties of the cyclodextrins enable the control over solubility of various formulation components by increasing or decreasing their solubility.

Numerous cyclodextrins and methods for their preparation have been described. For example, Parmeter (I), et al. (U.S. Pat. No. 3,453,259) and Gramera, et al. (U.S. Pat. No. 3,459,731) described electroneutral cyclodextrins. Other derivatives include cyclodextrins with cationic properties [Parmeter (II), U.S. Pat. No. 3,453,257], insoluble crosslinked cyclodextrins (Solms, U.S. Pat. No. 3,420,788), and cyclodextrins with anionic properties [Parmeter (III), U.S. Pat. No. 3,426,011]. Among the cyclodextrin derivatives with anionic properties, carboxylic acids, phosphorous acids, phosphinous acids, phosphonic acids, phosphoric acids, thiophosphonic acids, thiosulphinic acids, and sulfonic acids have been appended to the parent cyclodextrin [see, Parmeter (III), supra]. Furthermore, sulfoalkyl ether cyclodextrin derivatives have been described by Stella, et al. (U.S. Pat. No. 5,134,127).

Liposomes consist of at least one lipid bilayer membrane enclosing an aqueous internal compartment. Liposomes may be characterized by membrane type and by size. Small unilamellar vesicles (SUVs) have a single membrane and typically range between 0.02 and 0.05 μm in diameter; large unilamellar vesicles (LUVS) are typically larger than 0.05 μm. Oligolamellar large vesicles and multilamellar vesicles have multiple, usually concentric, membrane layers and are typically larger than 0.1 μm. Liposomes with several nonconcentric membranes, i.e., several smaller vesicles contained within a larger vesicle, are termed multivesicular vesicles.

One aspect of the present technology relates to formulations comprising liposomes containing an oligomer of the present technology, where the liposome membrane is formulated to provide a liposome with increased carrying capacity. Alternatively or in addition, the compound of the present technology may be contained within, or adsorbed onto, the liposome bilayer of the liposome. An oligomer of the present technology may be aggregated with a lipid surfactant and carried within the liposome's internal space; in these embodiments, the liposome membrane is formulated to resist the disruptive effects of the active agent-surfactant aggregate.

According to one embodiment of the present technology, the lipid bilayer of a liposome contains lipids derivatized with polyethylene glycol (PEG), such that the PEG chains extend from the inner surface of the lipid bilayer into the interior space encapsulated by the liposome, and extend from the exterior of the lipid bilayer into the surrounding environment.

Active agents contained within liposomes of the present technology are in solubilized form. Aggregates of surfactant and active agent (such as emulsions or micelles containing the active agent of interest) may be entrapped within the interior space of liposomes according to the present technology. A surfactant acts to disperse and solubilize the active agent, and may be selected from any suitable aliphatic, cycloaliphatic or aromatic surfactant, including but not limited to biocompatible lysophosphatidylcholines (LPGs) of varying chain lengths (for example, from about C14 to about C20). Polymer-derivatized lipids such as PEG-lipids may also be utilized for micelle formation as they will act to inhibit micelle/membrane fusion, and as the addition of a polymer to surfactant molecules decreases the CMC of the surfactant and aids in micelle formation. Preferred are surfactants with CMOs in the micromolar range; higher CMC surfactants may be utilized to prepare micelles entrapped within liposomes of the present technology.

Liposomes according to the present technology may be prepared by any of a variety of techniques that are known in the art. See, e.g., U.S. Pat. No. 4,235,871; Published PCT applications WO 96/14057; New RRC, Liposomes: A practical approach, IRL Press, Oxford (1990), pages 33-104; Lasic D, Liposomes from physics to applications, Elsevier Science Publishers BV, Amsterdam, 1993. For example, liposomes of the present technology may be prepared by diffusing a lipid derivatized with a hydrophilic polymer into preformed liposomes, such as by exposing preformed liposomes to micelles composed of lipid-grafted polymers, at lipid concentrations corresponding to the final mole percent of derivatized lipid which is desired in the liposome. Liposomes containing a hydrophilic polymer can also be formed by homogenization, lipid-field hydration, or extrusion techniques, as are known in the art.

In another exemplary formulation procedure, the active agent is first dispersed by sonication in a lysophosphatidylcholine or other low CMC surfactant (including polymer grafted lipids) that readily solubilizes hydrophobic molecules. The resulting micellar suspension of active agent is then used to rehydrate a dried lipid sample that contains a suitable mole percent of polymer-grafted lipid, or cholesterol. The lipid and active agent suspension is then formed into liposomes using extrusion techniques as are known in the art, and the resulting liposomes separated from the unencapsulated solution by standard column separation.

In one aspect of the present technology, the liposomes are prepared to have substantially homogeneous sizes in a selected size range. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane. Sec e.g., U.S. Pat. No. 4,737,323 (Apr. 12, 1988). In some embodiments, reagents such as DharmaFECT® and Lipofectamine® may be utilized to introduce polynucleotides or proteins into cells.

The release characteristics of a formulation of the present technology depend on the encapsulating material, the concentration of encapsulated drug, and the presence of release modifiers. For example, release can be manipulated to be pH dependent, for example, using a pH sensitive coating that releases only at a low pH, as in the stomach, or a higher pH, as in the intestine. An enteric coating can be used to prevent release from occurring until after passage through the stomach. Multiple coatings or mixtures of cyanamide encapsulated in different materials can be used to obtain an initial release in the stomach, followed by later release in the intestine. Release can also be manipulated by inclusion of salts or pore forming agents, which can increase water uptake or release of drug by diffusion from the capsule. Excipients which modify the solubility of the drug can also be used to control the release rate. Agents which enhance degradation of the matrix or release from the matrix can also be incorporated. They can be added to the drug, added as a separate phase (i.e., as particulates), or can be co-dissolved in the polymer phase depending on the compound. In most embodiments the amount should be between 0.1 and thirty percent (w/w polymer). Types of degradation enhancers include inorganic salts such as ammonium sulfate and ammonium chloride, organic acids such as citric acid, benzoic acid, and ascorbic acid, inorganic bases such as sodium carbonate, potassium carbonate, calcium carbonate, zinc carbonate, and zinc hydroxide, and organic bases such as protamine sulfate, spermine, choline, ethanolamine, diethanolamine, and triethanolamine and surfactants such as Tween® and Pluronic®. Pore forming agents which add microstructure to the matrices (i.e., water soluble compounds such as inorganic salts and sugars) are added as particulates. The range is typically between one and thirty percent (w/w polymer).

Uptake can also be manipulated by altering residence time of the particles in the gut. This can be achieved, for example, by coating the particle with, or selecting as the encapsulating material, a mucosal adhesive polymer. Examples include most polymers with free carboxyl groups, such as chitosan, celluloses, and especially polyacrylates (as used herein, polyacrylates refers to polymers including acrylate groups and modified acrylate groups such as cyanoacrylates and methacrylates).

An oligomer may be formulated to be contained within, or, adapted to release by a surgical or medical device or implant. In some aspects, an implant may be coated or otherwise treated with an oligomer. For example, hydrogels, or other polymers, such as biocompatible and/or biodegradable polymers, may be used to coat an implant with the compositions of the present technology (i.e., the composition may be adapted for use with a medical device by using a hydrogel or other polymer). Polymers and copolymers for coating medical devices with an agent are well-known in the art. Examples of implants include, but are not limited to, stents, drug-eluting stents, sutures, prosthesis, vascular catheters, dialysis catheters, vascular grafts, prosthetic heart valves, cardiac pacemakers, implantable cardioverter defibrillators, IV needles, devices for bone setting and formation, such as pins, screws, plates, and other devices, and artificial tissue matrices for wound healing.

In addition to the methods provided herein, the oligomers for use according to the present technology may be formulated for administration in any convenient way for use in human or veterinary medicine, by analogy with other pharmaceuticals. The ASOs and their corresponding formulations may be administered alone or in combination with other therapeutic strategies in the treatment of muscular dystrophy, such as myoblast transplantation, stem cell therapies, administration of aminoglycoside antibiotics, proteasome inhibitors, and up-regulation therapies (e.g., upregulation of utrophin, an autosomal paralogue of dystrophin).

The routes of administration described are intended only as a guide since a skilled practitioner will be able to determine readily the optimum route of administration and any dosage for any particular animal and condition. Multiple approaches for introducing functional new genetic material into cells, both in vitro and in vivo have been attempted (Theodore Friedmann. Progress Toward Human Gene Therapy. 1989. Science. 244:1275-1280. doi: 10.1126/science. 266025). These approaches include integration of the gene to be expressed into modified retroviruses (Theodore Friedmann (1989) supra; Steven A. Rosenberg. Immunotherapy and Gene Therapy of Cancer. 1991 Sep. 15. Cancer Research. 51 (18), suppl.: 5074S-5079S.); integration into non-retrovirus vectors (e.g., adeno-associated viral vectors) (M. A. Rosenfeld, et al. In vivo transfer of the human cystic fibrosis transmembrane conductance regulator gene to the airway epithelium. 1992 Jan. 10. Cell. 68:143-155. doi: 10.1016/0092-8674 (92) 90213-V; M. A. Rosenfeld, et al. Adenovirus-Mediated Transfer of a Recombinant al-Antitrypsin Gene to the Lung Epithelium in Vivo. 1991 Apr. 19. Science. 252:431-434. doi: 10.1126/science. 2017680); or delivery of a transgene linked to a heterologous promoter-enhancer element via liposomes (Theodore Friedmann (1989), supra; Kenneth L. Brigham, et al. Rapid Communication: In vivo Transfection of Murine Lungs with a Functioning Prokaryotic Gene using a Liposome Vehicle. 1989 October. Am. J. Med. Sci. 298:278-281. doi: 10.1097/00000441-198910000-00013; Elizabeth G. Nabel, et al. Site-Specific Gene Expression in Vivo by Direct Gene Transfer into the Arterial Wall. 1990 September. Science. 249:1285-1288. doi: 10.1126/science. 211905; Thomas A. Hazinski, et al. Localization and Induced Expression of Fusion Genes in the Rat Lung. 1991 November. Am. J. Resp. Cell Molec. Biol. 4:206-209. doi: 10.1165/ajrcmb/4.3.206; and Wang and Huang. pH-sensitive immunoliposomes mediate target-cell-specific delivery and controlled expression of a foreign gene in mouse. 1987 Nov. 11. Proc. Natl. Acad. Sci. USA. 84:7851-7855. doi: 10.1073/pnas. 84.22.7851); coupled to ligand-specific, cation-based transport systems (Wu and Wu. Receptor-mediated gene delivery and expression in vivo. 1988 October. J. Biol. Chem. 263:14621-14624. doi: 10.1016/S0021-9258 (18) 68081-0) or the use of naked DNA, expression vectors (Elizabeth G. Nabel, et al. (1990), supra); Jon A. Wolff, et al. Direct Gene Transfer into Mouse Muscle in Vivo. 1990 Mar. 23. Science. 247:1465-1468. doi: 10.1126/science. 1690918). Direct injection of transgenes into tissue produces only localized expression (M. A. Rosenfeld (1992) supra); M. A. Rosenfeld, et al. (1991) supra; Kenneth L. Brigham, et al. (1989) supra; Elizabeth G. Nabel (1990) supra; and Thomas A. Hazinski, et al. (1991) supra). The Brigham et al. group (Rapid Communication: In vivo Transfection of Murine Lungs with a Functioning Prokaryotic Gene using a Liposome Vehicle. 1989 October. Am. J. Med. Sci. 298:278-281. doi: 10.1097/00000441-198910000-00013 and Clinical Research (1991) 39 (abstract)) have reported in vivo transfection only of lungs of mice following either intravenous or intratracheal administration of a DNA liposome complex. An example of a review article of human gene therapy procedures is W. F. Anderson. Human gene therapy. 1992 May 8. Science. 256:808-813. doi: 10.1126/science. 1589762.

### Combination Therapy

“Co-administration” or “co-administering” or “combination therapy” as used herein generally refers to the administration of one or more ASOs of the present technology (e.g., bipartite ASOs) in conjunction with other remedies known in the art that are used to treat the same disease the ASOs treat or complications thereof.

In certain embodiments, the disease is DMD and the other remedies treat muscular dystrophy or its complications, including but not limited to: corticosteroids (e.g., cortisol, hydrocortisone, prednisone, prednisolone, deflazacort, triamcinolone, methylprednisolone, dexamethasone, betamethasone, aldosterone, and fludrocortisone); β2-adrenergic agonists (e.g., albuterol, salbutamol, levosalbutamol, terbutaline, pirbuterol, procaterol, clenbuterol, metaproterenol, fenoterol, bitolterol mesylate, ritodrine, isoprenaline, salmeterol, formoterol, bambuterol, and indicaterol); immunosuppressants (e.g., cyclosporine); anti-fibrotic drugs (e.g., peginterferon, IL-10, pioglitazone, pentoxifylline, atanercept); exon-skipping drugs (e.g., ASOs; for example, ASOs that target exon 51, exon 45, or exon 53 including drisapersen, eteplirsen, golodirsen, PRO044, PRO45, PRO051, and PRO053); stop-codon skipping drugs, (e.g., gentamycin or other aminoglycoside antibiotics and Ataluren (PTC124)); synthetic anabolic steroids (e.g., oxandrolone); osteoporosis remedies (e.g., vitamin D and calcium); constipation remedies including laxatives; cardiomyopathy remedies including angiotensin-converting enzyme (ACE) inhibitors (e.g., benazepril, captopril, enalapril, fosinopril, lisinopril, moexipril, perindopril, quinapril, ramipril, and trandolapril), diuretics, beta-blockers (e.g., bisoprolol or carvedilol), anti-arrhythmic medications (e.g., amiodarone); insulin-like growth factor (IGF-1); myostatin inhibitors (e.g., follistatin, ACE-031, and neutralizing antibodies including MYO-029); drugs that increase nitric oxide levels and/or nNOS protein levels or activity (e.g., L-arginine; phosphodiesterase inhibitors including sildenafil, tadalafil, and pentoxifyllinc); class II histone deacetylase (HDAC) inhibitors; small molecules that increase utrophin expression (e.g., SMT C1100); nutritional supplements (e.g., glutamine, creatine monohydrate, conjugated linoleic acid, alpha-lipoic acid, and beta-hydroxy-beta-methylbutyrate); anti-histamines (e.g., fexofenadine, loratadine, phenindamine, dexchlorpheniramine, terfenadine, cetirizine, etc.); mast cell stabilizers (e.g., sodium cromoglicate, nedocromil sodium, etc., which can be in a form of aerosols, inhalations, eye drops, etc.); coenzyme Q10 (also known as ubiquinone or ubidecarenone); idebenone or other synthetic derivatives of ubidecarenone, (e.g., RAXONE®/CATENA®); omega 3; resveratrol; phytosterols/stanols; anticoagulants (e.g., warfarin); and anticholinergic agents (e.g., anti-muscarinic agents (e.g., atropine, benztropine (COGENTIN®), biperiden, chlorpheniramine (CHLOR-TRIMETON), dicyclomine (dicycloverine), dimenhydrinate (DRAMAMINE®), diphenhydramine (BENADRYL®, SOMINEX™, ADVIL®, PM, etc.), doxylamine (UNISOM™), glycopyrrolate (ROBINUL®), ipratropium (ATROVENT®), orphenadrine, oxitropium (OXIVENT®), oxybutynin (DITROPAN®, DRIPTANE®, LYRINEL® XL), tolterodine (DETROL®, DETRUSITOL), tiotropium (SPIRIVA®), trihexyphenidyl, scopolamine, solifenacin, and tropicamide), and anti-nicotinic agents (e.g., ganglion blockers including bupropion (ZYBAN®, WELLBUTRIN®) and hexamethonium, cough suppressants and ganglion blockers (e.g., dextromethorphan), nondepolarizing skeletal muscular relaxants (e.g., doxacurium and tubocurarine), ganglion blockers and occasional smoking cessation aids (e.g., mecamylamine)), which can be in a form of inhalers, nebulizer solutions, tablets, and can be administered by rectal, oral, transdermal, or parenteral routes). The compositions and methods of the present technology can also be used with, for example, other gene-based therapy approaches (e.g., viral delivery of mini- or micro-dystrophin, mini-utrophin, or trans-splicing recombinant AAV vectors); gene editing, including approaches involving zinc-finger nucleases, transcription activator-like (TAL) type III effector nucleases (TALENs), meganucleases, or clustered regularly interspaced short palindromic repeats (CRISPR), or with cell-based therapies involving transplantation of various types of precursor cells, such as ex vivo-manipulated muscle side population cells (alineage of uncommitted cells) into muscle fibers. The compositions and methods can also be used with any of the therapies described in Pedro Miura, et al. Utrophin upregulation for treating Duchenne or Becker muscular dystrophy: how close are we? 2006 March. Trends. Mol. Med. 12:122-129. doi: 10.1016/j. molmed. 2006.01.002; Susan Jarmin, et al. New developments in the use of gene therapy to treat Duchenne muscular dystrophy. 2013 Dec. 6. Expert Opin. Biol. Ther. 14:209-230. doi: 10.1517/14712598.2014.866087; M. A. Scully, et al. Review of Phase II and Phase III clinical trials for Duchenne muscular dystrophy. 2012 Dec. 17. Expert Opin. Orphan Drugs. 1:33-46. doi: 10.1517/21678707.2013.746939; R. J. Fairclough, et al. Progress in therapy for Duchenne muscular dystrophy. 2011 Jul. 29. Exp. Physiol. 96:1101-1113. doi: 10.1113/expphysiol. 2010.053025; and Michael J. Blankinship, et al. Gene Therapy Strategies for Duchenne Muscular Dystrophy Utilizing Recombinant Adeno-associated Virus Vectors. 2006 February. Mol. Therapy. 13:241-249. doi: 10.1016/j. ymthe. 2005.11.001.

In some embodiments, one or more ASOs of the present technology are administered in a pharmaceutically acceptable dosage form in combination with one or more additional therapeutic agents. Each therapeutic agent in a combination therapy disclosed herein may be administered either alone or in a medicament (also referred to herein as a pharmaceutical composition) which comprises the therapeutic agent and one or more pharmaceutically acceptable carriers, excipients and diluents, according to standard pharmaceutical practice. Each therapeutic agent may be prepared by formulating a compound or pharmaceutically acceptable salt thereof separately, and both may be administered either at the same time or separately. Further, the two formulations may be placed in a single package, to provide the kit formulation. In some embodiments, both compounds may be contained in a single formulation. In some embodiments, the therapeutic agents are in the same dosage form, e.g., the same tablet or pharmaceutical composition. In some embodiments, the therapeutic agents are in separate dosage forms having the same mode of administration, e.g., a kit comprising a first pharmaceutical composition suitable for parenteral administration comprising an ASO and a pharmaceutically acceptable carrier, a second pharmaceutical composition suitable for parenteral administration comprising one or more additional therapeutic agents. In some embodiments, the therapeutic agents are in separate dosage forms having different modes of administration, e.g., a kit comprising a first pharmaceutical composition suitable for parenteral administration comprising an ASO, and a pharmaceutically acceptable carrier, a second pharmaceutical composition suitable for oral administration comprising one or more additional therapeutic agents, and optionally a third pharmaceutical composition suitable for oral administration comprising one or more other therapeutic agents.

Each therapeutic agent in a combination therapy disclosed herein may be administered simultaneously (i.e., in the same medicament), concurrently (i.e., in separate medicaments administered one right after the other in any order) or sequentially in any order. Sequential administration is useful when the therapeutic agents in the combination therapy are in different dosage forms (e.g., one agent is a tablet or capsule and another agent is a sterile liquid) and/or are administered on different dosing schedules, e.g., tablet or capsule formulated for daily administrat ion and a composition formulated for parenteral administration, such as once weekly, once every two weeks, or once every three weeks. Further, those of skill in the art given the benefit of the present technology will appreciate that when more than one therapeutic agents disclosed herein are being administered, the agents need not share the same mode of administration, e.g., a kit comprising a first pharmaceutical composition suitable for parenteral administration comprising an ASO and a pharmaceutically acceptable carrier, a second pharmaceutical composition suitable for oral administration comprising an additional therapeutic agent disclosed herein and a pharmaceutically acceptable carrier. Those of skill in the art will appreciate that the concomitant administration referred to above in the context of “co-administering” or “co-administration” or “combination therapy” means that the pharmaceutical composition comprising DMD exon-skipping ASO and a pharmaceutical composition(s) comprising additional therapeutic agent(s) can be administered on the same schedule, i.e., at the same time and day, or on a different schedule, i.e., on different, although not necessarily distinct, schedules. Other suitable variations to “co-administering”, “co-administration” or “combination therapy” will be readily apparent to those of skill in the art given the benefit of the present technology and are part of the meaning of these terms.

The compositions and methods of the present technology can also be used in conjunction with other forms of treatment including but not limited to: physical exercise (e.g., physical therapy, range-of-motion exercises); mobility aids, supports or orthotic devices, (e.g., ankle splints, knee-ankle-foot orthosis (KAFO), spinal braces, and wheelchairs); breathing assistance (e.g., ventilators); and surgical remedies (e.g., tendon surgery, scoliosis surgery, installation of a pacemaker, and cardiac transplantation). The choice of specific treatment may vary and will depend upon the severity of the pain, the subject's general health, and the judgment of the attending clinician.

### Kit Comprising Bipartite ASOs

In some aspects, the present technology provides a kit comprising the ASO (e.g., bipartite ASO) disclosed herein, the composition (e.g., pharmaceutical composition) comprising the ASO (e.g., bipartite ASO) as disclosed herein, and/or the vector encoding the ASO (e.g., bipartite ASO) as disclosed herein for use in generating or promoting exon skipping of an exon of interest. The ASO-mediated exon skipping may be an approach to manipulate expression of a gene of interest. The expression of the gene of interest may be manipulated by ASOs to inhibit splicing of an exon, intron or a specific splice site of a gene of interest, leading to, e.g., without limitation, restoring the reading frame of a defective gene of interest, generating a different isoform of a gene of interest (e.g., a dominant negative isoform), skipping a toxic part of the gene, silencing the gene, and/or changing the structure and function of a gene. Examples of the exon of interest and gene of interest include, without limitation, those disclosed herein.

In some aspects, the present technology provides a kit comprising the ASO (e.g., bipartite ASO) disclosed herein, the composition (e.g., pharmaceutical composition) comprising the ASO (e.g., bipartite ASO) as disclosed herein, and/or the vector encoding the ASO (e.g., bipartite ASO) as disclosed herein for use in treating a disease and/or complications thereof. Examples of the disease and/or complications thereof include, without limitation, those disclosed herein.

### EXAMPLES

The following Examples provide an additional description of the present technology for illustrative purposes only and should not be construed to limit the scope of the present technology in any way.

### Example 1: Examples of Bipartite ASOs

Embodiments of the present technology include bipartite ASOs comprising a targeting sequence and a decoy sequence (see e.g., FIGS. 1A and 1B). The targeting sequence binds to a sequence at an exon of interest, a flanking intron sequence upstream of the exon of interest, a flanking intron sequence downstream of the exon of interest, an intron-exon junction upstream of the exon of interest, or an intron-exon junction downstream of the exon of interest with full or partial complementarity. The decoy sequence, constituting all or part of the 11-nt sequence (5′-CAGGTAAGTAT-3′), is fully complementary to the free 5′ end of U1 snRNA. Therefore, the decoy sequence, simulating an optimal 5′ splice site, may act as a 5′-splice-site decoy to interfere with the recognition of the authentic 5′ splice site of the exon of interest by U1 snRNA. In certain embodiments, the decoy sequence may have a length of 12 or 13 nt, e.g., as shown in Example 5. Without being bound by any particular theory, the targeting sequence may not only cause exon skipping to some extent by itself through steric blocking but also bring the decoy sequence to near the 5′ splice site of the exon of interest. The decoy sequence may resemble and simulate an optimal 5′ splice site that interferes with the recognition of the authentic 5′ splice site by U1 snRNA by potentially direct interaction with U1 snRNA. In certain examples, the targeting sequence and the decoy sequence act in concert to induce robust exon skipping. Although both ends of a bipartite ASO may comprise the same or different decoy sequence(s), bipartite ASOs tested in Examples 2-15 comprise a decoy positioned at either the 5′ or the 3′ end of a bipartite ASO (e.g., as shown in FIG. 1A). A bipartite ASO may exert its function by binding to the exon of interest, its flanking intron sequences, or intron-exon junctions via its targeting portion based on Watson-Crick base-pairing (e.g., as shown in FIG. 1B). In certain examples, the length of an optimal decoy sequence may vary from 6 to 11 nt. See Table 1 for nomenclature and sequences of all tested decoys.

Unless otherwise specified, in the Example section, L in a decoy name or an ASO name means the decoy is positioned at the 5′ end of a bipartite ASO; and R in a decoy name or an ASO name means the decoy is positioned at the 3′ end of a bipartite ASO. For example, L6a means that the 6a decoy sequence is positioned at the 5′ end of a bipartite ASO and R6a means that the 6a decoy sequence is positioned at the 3′ end of a bipartite ASO.

All ASOs tested in Examples 2-15 were 2′-O-methoxyethyl (MOE)-modified with phosphorothioate (PS) backbone and all cytosines were 5-methyl cytosines. Similar ASOs having other uniform modifications (e.g., without limitation, morpholino with phosphorodiamidate and constrained-ethyl (cEt)) or mixed modifications should also work.

TABLE 1
The 5′-splice-site decoy sequences tested in Examples 2-15

| Decoy name | | | | | | |
|---|---|---|---|---|---|---|
| | At the | At the | | | | Targeted sequence (underlined) |
| | 5′ end | 3′ end | | | Length | shown in the first 14-nt sequence at |
| Decoy | of ASO | of ASO | SEQ ID NO. | Sequence (5′ to 3′) | (nt) | the 5′ end of Ul snRNA (5′ to 3′) |
| 5a | L5a | R5a | SEQ ID No. | CAGGT | 5 | AUACUUACCUGGCA |
| | | | 347 | | | |
| 5b | L5b | R5b | SEQ ID No. | AGGTA | 5 | AUACUUACCUGGCA |
| | | | 348 | | | |
| 5c | L5c | R5c | SEQ ID No. | GOTAA | 5 | AUACUUACCUGGCA |
| | | | 349 | | | |
| 5d | L5d | R5d | SEQ ID No. | GTAAG | 5 | AUACUUACCUGGCA |
| | | | 350 | | | |
| 5e | LSe | RSe | SEQ ID No. | TAAGT | 5 | AUACUUACCUGGCA |
| | | | 351 | | | |
| 5f | L5f | R5f | SEQ ID No. | AAGTA | 5 | AUACUUACCUGGCA |
| | | | 352 | | | |
| 5g | L5g | R5g | SEQ ID No. | AGTAT | 5 | AUACUUACCUGGCA |
| | | | 353 | | | |
| 6a | L6a | R6a | SEQ ID No. | CAGGTA | 6 | AUACUUACCUGGCA |
| | | | 1 | | | |
| | | | | | Length | Targeted sequence (underlined) |
| Decoy name | | | SEQ ID No. | Sequence (5′ to 3′) | (nt) | shown in the first 14-nt sequence at |
| 6b | L6b | R6b | SEQ ID No. | AGGTAA | 6 | AUACUUACCUGGCA |
| | | | 2 | | | |
| 6c | L6c | R6c | SEQ ID No. | GGTAAG | 6 | AUACUUACCUGGCA |
| | | | 3 | | | |
| 6d | L6d | R6d | SEQ ID No. | GTAAGT | 6 | AUACUUACCUGGCA |
| | | | 4 | | | |
| | L6e | R6e | SEQ ID No. | TAAGTA | 6 | AUACUUACCUGGCA |
| | | | 5 | | | |
| 6f | L6f | R6f | SEQ ID No. | AAGTAT | 6 | AUACUUACCUGGCA |
| | | | 6 | | | |
| 7a | L7a | R7a | SEQ ID No. | CAGGTAA | 7 | AUACUUACCUGGCA |
| | | | 7 | | | |
| 7b | L7b | R7b | SEQ ID No. | AGGTAAG | 7 | AUACUUACCUGGCA |
| | | | 8 | | | |
| 7c | L7c | R7c | SEQ ID No. | GGTAAGT | 7 | AUACUUACCUGGCA |
| | | | 9 | | | |
| 7d | L7d | R7d | SEQ ID No. | GTAAGTA | 7 | AUACUUACCUGGCA |
| | | | 10 | | | |
| 7e | L7e | Re | SEQ ID No. | TAAGTAT | 7 | AUACUUACCUGGCA |
| | | | 11 | | | |
| 8a | L8a | R8a | SEQ ID No. | CAGGTAAG | 8 | AUACUUACCUGGCA |
| | | | 12 | | | |
| 8b | L8b | R8b | SEQ ID No. | AGGTAAGT | 8 | AUACUUACCUGGCA |
| | | | 13 | | | |
| 8c | L8c | R8c | SEQ ID NO. | GGTAAGTA | 8 | AUACUUACCUGGCA |
| | | | 14 | | | |
| 8d | L8d | R8d | SEQ ID No. | GTAAGTAT | 8 | AUACUUACCUGGCA |
| | | | 15 | | | |
| 19a | L9a | R9a | SEQ ID No. | CAGGTAAGT | 9 | AUACUUACCUGGCA |
| | | | 16 | | | |
| 9b | L9b | R9b | SEQ ID No. | AGGTAAGTA | 9 | AUACUUACCUGGCA |
| | | | 17 | | | |
| 9c | L9c | R9c | SEQ ID No. | GGTAAGTAT | 9 | AUACUUACCUGGCA |
| | | | 18 | | | |
| 10a | L10a | R10a | SEQ ID No. | CAGGTAAGTA | 10 | AUACUUACCUGGCA |
| | | | 19 | | | |
| 10b | L10b | R10b | SEQ ID No. | AGGTAAGTAT | 10 | AUACUUACCUGGCA |
| | | | 20 | | | |
| 11 | L11 | R11 | SEQ ID No. | CAGGTAAGTAT | 11 | AUACUUACCUGGCA |
| | | | 21 | | | |
| 12 | L12 | R12 | SEQ ID No. | CCAGGTAAGTAT | 12 | AUACUUACCUGGCA |
| | | | 22 | | | |
| 13 | L13 | R13 | SEQ ID No. | GCCAGGTAAGTAT | 13 | AUACUUACCUGGCA |
| | | | 23 | | | |

### Example 2: Testing ASOs for Exon Skipping Efficiencies

HEK293 cells, RD cells, HeLa cells, or A549 cells, were transfected with an ASO (12.5, 25, 40, or 50 nM) as listed in Tables 2-12 to test exon skipping efficiencies of the ASO in vitro. Each ASO was transfected into desired cells using Lipofectamine 2000, and buffer alone was used as a negative control. Two days post transfection, cells were collected, and total RNA samples were isolated for splicing analysis by semi-quantitative fluorescent RT-PCR.

Some ASO targeting sequences listed here were used to demonstrate that bipartite ASOs comprising a decoy and the corresponding targeting sequence are much more potent in inhibiting exon splicing than the targeting sequence alone. All ASOs were 2′-O-methoxyethyl (MOE)-modified with phosphorothioate (PS) backbone and all 5-methyl cytosines (5mC). Etep is an abbreviation for eteplirsen. Note, eteplirsen used here was also modified with MOE/PS/5mC instead of its original morpholino/phosphor odiamidate modifications.

### Example 3: ASO Induced Exon 7 Skipping of SMN1 and SMN2 Genes

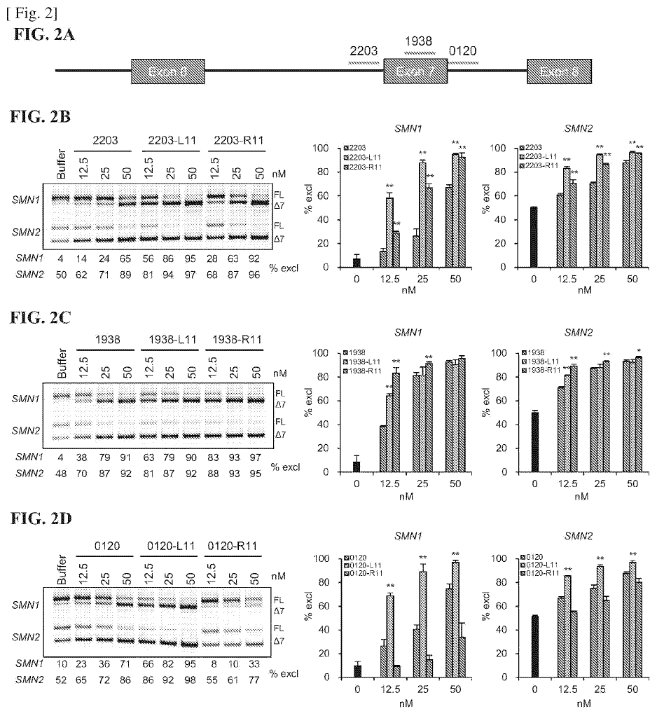

A 11-nt decoy sequence (e.g., 5′-CAGGTAAGTAT-3′) enhanced the effects of three ASO targeting sequences (2203, 1938 and 0120) on exon 7 skipping of the endogenous SMN1 and SMN2 genes in HEK293 cells with statistical significance, for example, as shown in FIGS. 2A-2D. FIG. 2A shows a schematic diagram of the region of interest of the SMN1/2 genes. Three ASO targeting sequences bind to their SMN1/2 pre-mRNA target sequences with full complementarity. ASO targeting sequence 2203 targets a 20-nt sequence from position-22 to-3 in intron 6, ASO targeting sequence 1938 targets a 20-nt sequence from position 19 to 38 in exon 7, and ASO targeting sequence 0120 targets a 20-nt sequence from position 1 to 20 in intron 7. The effects of ASO targeting sequence 2203 on promoting exon 7 skipping was increased when a decoy was attached to the 5′ end of ASO targeting sequence 2203 (2203-L11) or the 3′ end of ASO targeting sequence 2203 (2203-R11), as shown in FIG. 2B. Each ASO at 12.5, 25 or 50 nM was transfected into HEK293 cells using Lipofectamine 2000, and buffer alone was used as a negative control. Two days post transfection, cell samples were collected, and total RNA was purified for splicing analysis by semi-quantitative fluorescent RT-PCR (Y. Gao, et al. Systematic characterization of short intronic splicing-regulatory elements in SMN2 pre-mRNA. 2022 Jan. 8. Nucleic Acids Research. 50:731-749. doi: 10.1093/nar/gkab1280). The PCR products were digested with DdeI to distinguish their origin (SMN1 or SMN2). The percentage of exon 7 exclusion (% excl) in total transcripts of each gene was calculated. Quantitation of the data (n=3) is shown in the middle panel (SMN1) and the right panel (SMN2) of FIG. 2B. The effects of ASO targeting sequence 1938 on promoting exon 7 skipping was increased when a decoy was attached to the 5′ end of ASO targeting sequence 1938 (1938-L11) or the 3′ end of ASO targeting sequence 1938 (1938-R11), as shown in FIG. 2C. The experimental procedure for assessing the effects of ASOs comprising targeting sequence 1938 was the same as the experimental procedure for assessing the effects of ASOs comprising targeting sequence 2203. The effects of ASO targeting sequence 0120 on promoting exon 7 skipping was increased when a decoy was attached to the 5′ end of ASO 0120 (0120-L11) or the 3′ end of ASO 0120 (0120-R11), as shown in FIG. 2D. The experimental procedure for assessing the effects of ASOs comprising targeting sequence 0120 was the same as the experimental procedure for assessing the effects of ASOs comprising targeting sequence 2203 and ASOs comprising targeting sequence 0120. Representatives of three independent experiments for ASOs comprising targeting sequence 2203 (2203, 2203-L11, 2203-R11), ASOs comprising targeting sequence 1938 (1938, 1938-L11, 1938-R11), and ASOs comprising targeting sequence 0120 (0120, 0120-L11, 0120-R11) are shown in the left panels of FIGS. 2B-2D, respectively (FL: full-length transcript, Δ7: exon 7-skipped transcript.). Quantitation of data (n=3) for ASOs comprising targeting sequence 2203, 1938, or 0120 is shown in the middle panels (SMN1) and the right panels (SMN2) of FIGS. 2B-2D, respectively, as mean±standard deviation. *P<0.05, **P<0.01 compared to the corresponding ASO comprising targeting sequence only, e.g., 2203, 1938 or 0120. All ASOs are 2′-O-methoxyethyl-modified with phosphorothioate backbone and all 5-methyl cytosines. ASOs 2203-L11, 1938-L11, 1938-R11, and 0120-L11 are optimal ASOs. See Table 1 for sequence information of decoy sequences and Table 2 for sequence information of ASOs and targeting sequences tested in Example 3. The targeted sequence (5′ to 3′) in SMN1/2 mRNA complementary to ASO targeting sequence 2203 is ACUUCCUUUAUUUUCCUUAC (SEQ ID No. 24), the targeted sequence (5′ to 3′) in SMN1/2 mRNA complementary to ASO targeting sequence 1938 is AAAGAAGGAAGGUGCUCACA (SEQ ID No. 28), and the targeted sequence (5′ to 3′) in SMN1/2 mRNA complementary to ASO targeting sequence 0120 is GUAAGUCUGCCAGCAUUAUG (SEQ ID No. 32).

As shown in FIGS. 2B-2D, an ASO comprising the decoy sequence 11 attached to a targeting sequence transfected at a lower concentration may achieve a comparable 9% excl of exon 7 of SMN1/2 to an ASO consisting of the targeting sequence transfected at a higher concentration. As shown in the left panel of FIG. 2B, 2203-L11 (12.5 nM) achieved a comparable % excl of exon 7 of SMN1 to the targeting sequence 2203 (50 nM), and the % excl of exon 7 of SMN1 of 2203-L11 (12.5 nM) was about 2.3 times that of the targeting sequence 2203 (25 nM) and 4 times that of the targeting sequence 2203 at the same concentration. Similar results were observed in 2203-R11 vs 2203. Half dose of 2203-R11 (12.5 nM, 25 nM) achieved a comparable % excl of exon 7 of SMN1 to the targeting sequence 2203 (25 nM, 50 nM), respectively. 2203-R11 (12.5 nM) showed a comparable performance to the targeting sequence 2203 (25 nM) on skipping of exon 7 of the SMN2 gene. As shown in the left panel of FIG. 2C, at the same concentration 12.5 nM, the % excl of exon 7 of SMN1 of 1938-R11 was about 2.18 times that of the targeting sequence 1938, which was comparable to the targeting sequence 1938 (25 nM). FIG. 2D showed that 0120-L11 (12.5 nM) achieved a comparable % excl of exon 7 of SMN1 to the targeting sequence 0120 (50 nM).

Three 5-nt decoy sequences (AGGTA or 5b, GTAAG or 5d, and AGTAT or 5g) attached to either side of ASO targeting sequence 1938 were tested on SMN2 exon 7 splicing. ASO 1938-R5b (SEQ ID No. 354: 5′-TGTGAGCACCTTCCTTCTTTAGGTA-3′) was relatively strong and showed a skipping effect comparable to 1938-R11 (FIG. 2C), while 1938-R5d (SEQ ID No. 355: 5′-TGTGAGCACCTTCCTTCTTTGTAAG-3′) and 1938-R5g (SEQ ID No. 356: 5′-TGTGAGCACCTTCCTTCTTTAGTAT-3′) were moderate in promoting exon 7 skipping.

### Example 4: ASO Induced Exon 51 Skipping of DMD Gene

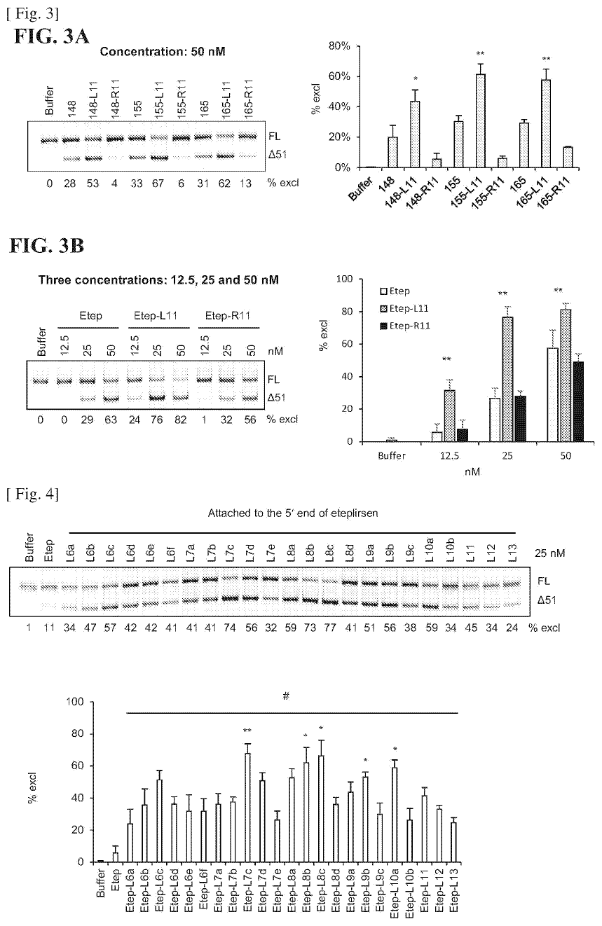

A 11-nt decoy sequence (e.g., 5′-CAGGTAAGTAT-3′) improved the effects of several ASO targeting sequences that promote exon 51 skipping of the endogenous DMD gene with statistical significance, for example, as shown in FIGS. 3A and 3B. Each ASO or ASO targeting sequence at indicated concentrations was transfected into RD cells using Lipofectamine 2000. Two days post transfection, cells were collected, and total RNA samples were isolated for splicing analysis by semi-quantitative fluorescent RT-PCR. As shown in FIG. 3A, in embodiments, ASO targeting sequences having decoy sequences on the 5′ end (148-L11, 155-L11, and 165-L11) had greater exon skipping effects than respective ASOs without decoy sequences (148, 155, and 165), but ASOs having decoy sequences on the 3′ end (148-R11, 155-R11, and 165-R11) showed minimal difference from respective ASO targeting sequences without decoy sequences. ASO targeting sequences 148, 155, and 165 target different regions in DMD exon 51 with full complementarity. As shown in FIG. 3B, in some embodiments, decoy sequences on ASO targeting sequences markedly improved the effect of the MOE-version eteplirsen (Etep) when placed at the 5′ end (Etep-L11) but not when placed at the 3′ end (Etep-R11). One representative of four independent experiments for ASOs comprising targeting sequence 148 (148, 148-L11, and 148-R11), ASOs comprising targeting sequence 155 (155, 155-L11, and 155-R11), and ASOs comprising targeting sequence 165 (165, 165-L11, and 165-R11) are shown in the left panel of FIG. 3A, and one representative gel of four independent experiments for ASOs comprising targeting sequence Etep (Etep, Etep-L11, Etep-R11) is shown in the left panels of FIG. 3B. FL: full-length transcript, A51: exon 51-skipped transcript.

Quantitation of exon 51 skipping data (n=4) for ASOs comprising targeting sequence 148, 155, 165, or Etep is shown on the right panels of FIGS. 3A-3B as mean±standard deviation. *P<0.05 (148-L11 vs 148); **P<0.01 (155-L11 vs 155, 165-L11 vs 165, or Etep-L11 vs Etep). ASOs 148-L11, 155-L11, 165-L11, Etep-L11, and Etep-R11 are optimal ASOs. See Table 1 for sequence information of decoy sequences and Table 2 for sequence information of ASOs and targeting sequences tested in Example 4. The targeted sequence (5′ to 3′) in DMD mRNA complementary to ASO targeting sequence 148 is AAUGCCAUCUUCCUUGAUG (SEQ ID No. 36), the targeted sequence (5′ to 3′) in DMD mRNA complementary to ASO targeting sequence 155 is AACUAGAAAUGCCAUCUUC (SEQ ID No. 40), the targeted sequence (5′ to 3′) in DMD mRNA complementary to ASO targeting sequence 165 is GCCAUCUCCAAACUAGAAA (SEQ ID No. 44), and the targeted sequence (5′ to 3′) in DMD mRNA complementary to ASO targeting sequence Etep is CUAGAAAUGCCAUCUUCCUUGAUGUUGGAG (SEQ ID No. 48).

The excl of exon 51 of DMD by Etep-L11 (12.5 nM) was comparable to the targeting sequence Etep (25 nM).

TABLE 2
The ASOs and targeting sequences tested in Examples 3 and 4

| | | | | | Effect in |
|---|---|---|---|---|---|
| ASO | | | Length | Targeted sequence (5′ to 3′) in | inducing cxon |
| name | SEQ ID No. | Sequence (5′ to 3′) | (nt) | SMN1/2 or DMD mRNA | skipping* |
| 2203 | SEQ ID No. 25 | GTAAGGAAAATAA | 20 | ACUUCCUUUAUUUUCCUUACA | + |
| | | AGGAAGT | | | |
| 2203- | SEQ ID No. 26 | CAGGTAAGTATGTA | 31 | ACUUCCUUUAUUUUCCUUAC | +-+ |
| L11 | | AGGAAAATAAAGG | | | |
| | | AAGT | | | |
| 2203- | SEQ ID No. 27 | GTAAGGAAAATAA | 31 | ACUUCCUUUAUUUUCCUUAC | |
| R11 | | AGGAAGTCAGGTA | | | |
| | | AGTAT | | | |
| 1938 | SEQ ID No. 29 | TGTGAGCACCTTCC | 20 | AAAGAAGGAAGGUGCUC | + |
| | | TTCTTT | | | |
| 1938- | SEQ ID No. 30 | CAGGTAAGTATTOT | 31 | AAAGAAGGAAGGUGCUCACA | −++ |
| L11 | | GAGCACCTTCCTTC | | | |
| | | TTT | | | |
| 1938- | SEQ ID No. 31 | TGTGAGCACCTTCC | 31 | AAAGAAGGAAGGUGCUCACA | −++ |
| R11 | | TTCTTTCAGGTAAG | | | |
| | | TAT | | | |
| 0120 | SEQ ID No. 33 | CATAATGCTGGCAG | 20 | GUAAGUCUGCCAGCAUUAUG | − |
| | | ACTTAC | | | |
| 0120- | SEQ ID No. 34 | CAGGTAAGTATCAT | 31 | GUAAGUCUGCCAGCAUUAUG | −++ |
| L11 | | AATGCTGGCAGACT | | | |
| | | TAC | | | |
| 0120- | SEQ ID No. 35 | CATAATGCTGGCAG | 31 | GUAAGUCUGCCAGCAUUAUG | |
| R11 | | ACTTACCAGGTAAG | | | |
| | | TAT | | | |
| 148 | SEQ ID No. 37 | CATCAAGGAAGAT | 19 | AAUGCCAUCUUCCUUGAUG | |
| | | GGCATT | | | |
| 148- | SEQ ID No. 38 | CAGGTAAGTATCAT | 30 | AAUGCCAUCUUCCUUGAUG | ++ |
| L11 | | CAAGGAAGATGGCA | | | |
| | | TT | | | |
| 148- | SEQ ID No. 39 | CATCAAGGAAGATG | 30 | AAUGCCAUCUUCCUUGAUG | |
| R11 | | GCATTCAGGTAAGT | | | |
| | | AT | | | |
| 155 | SEQ ID No. 41 | GAAGATGGCATTTC | 19 | AACUAGAAAUGCCAUCUUC | − |
| | | TAGTT | | | |
| 155- | SEQ ID No. 42 | CAGGTAAGTATGA | 30 | AACUAGAAAUGCCAUCUUC | −++ |
| L11 | | AGATGGCATTTCTA | | | |
| | | GTT | | | |
| 155- | SEQ ID No. 43 | GAAGATGGCATTTC | 30 | AACUAGAAAUGGAUCUUC | |
| R11 | | TAGTTCAGGTAAGT | | | |
| | | AT | | | |
| 165 | SEQ ID No. 45 | TTTCTAGTTTGGAG | 19 | GCCAUCUCCAAACUAGAAA | − |
| | | ATGGC | | | |
| 165- | SEQ ID No. 46 | CAGGTAAGTATTTT | 30 | GCCAUCUCCAAACUAGAAA | −++ |
| L11 | | CTAGTTTGGAGATG | | | |
| | | GC | | | |
| 165- | SEQ ID No. 47 | TTTCTAGTTTGGAG | 30 | GCCAUCUCCAAACUAGAAA | |
| R11 | | ATGGCCAGGTAAGT | | | |
| | | AT | | | |
| Etep | SEQ ID No. 49 | CTCCAACATCAAGG | 30 | CUAGAAAUGCCAUCUUCC | ++ |
| | | AAGATGGCATTTCT | | UUGAUGUUGGAG | |
| | | AG | | | |
| Etep- | SEQ ID No. 50 | CAGGTAAGTATCTC | 41 | CUAGAAAUGCCAUCUUCC | −+++++ |
| L11 | | CAACATCAAGGAA | | UUGAUGUUGGAG | |
| | | GATGGCATTTCTAG | | | |
| Etep- | SEQ ID No. 51 | CTCCAACATCAAGG | 41 | CUAGAAAUGCCAUCUUCC | −+ |
| R11 | | AAGATGGCATTTCT | | UUGAUGUUGGAG | |
| | | AGCAGGTAAGTAT | | | |

*: The more “+” the better exon skipping effect.

### Example 5: ASO Induced Exon 51 Skipping of DMD Gene with Decoy Sequences of Varying Lengths Attached to the 5′ End (“L”) of Targeting Sequence Eteplirsen

Bipartite ASOs having decoy sequences of varying lengths may have effects of different degrees on the ability of the ASO to promote exon skipping in the DMD gene, as shown in, for example, FIG. 4. 23 5′-splice-site decoy sequences with lengths from 6 to 13 nt were tested in the context of targeting sequence eteplirsen (Etep). The 12-nt (L12) and 13-nt (L13) long decoy sequences tested were complementary to the first 12- and 13-nt sequence at the 5′ end of U1 snRNA, respectively. The decoy sequences were positioned at the 5′ end of the bipartite ASOs. All ASOs tested were MOE-modified with PS backbone. Each ASO at 25 nM was transfected into RD cells using Lipofectamine 2000, and buffer alone was used as a negative control. Two days post transfection, cells were collected, and total RNA samples were isolated for splicing analysis by semi-quantitative fluorescent RT-PCR. All bipartite ASOs tested displayed a stronger effect on exon 51 skipping of the DMD gene with statistical significance than eteplirsen alone. One representative of three independent experiments is shown in FIG. 4 (top panel, FL: full-length transcript, Δ51: exon 51-skipped transcript). Quantitation of data (n=3) is shown in the bottom panel of FIG. 4, as mean±standard deviation. ^{#}P<0.05 (all vs Etep); *P<0.05, **P<0.01 (all vs Etep-L11). ASOs Etep-L7c, Etep-L8b, Etep-L8c, Etep-L9b, and Etep-L10a are optimal ASOs (FIG. 4). See Table 1 for sequence information of decoy sequences and Table 3 for sequence information of ASOs and the targeting sequence tested in Example 5, the targeted sequence (5′ to 3′) in DMD exon 51 is CUAGAAAUGCCAUCUUCCUU GAUGUUGGAG (SEQ ID No. 48).

TABLE 3
The ASOs and the targeting sequence tested in Example 5

| | | | | Optimal |
|---|---|---|---|---|
| | | | | ASOs for |
| | | | Length | exon |
| ASO name | SEQ ID No. | Sequence (5′ to 3′) | (nt) | skipping |
| Etep | SEQ ID No. 49 | CTCCAACATCAAGGAAGATGGCATTTCTAG | 30 | |
| Etep-L6a | SEQ ID No. 52 | CAGGTACTCCAACATCAAGGAAGATGGC | 36 | |
| | | ATTTCTAG | | |
| Etep-L6b | SEQ ID No. 53 | AGGTAACTCCAACATCAAGGAAGATGGC | 36 | |
| | | ATTTCTAG | | |
| Etep-L6c | SEQ ID No. 54 | GGTAAGCTCCAACATCAAGGAAGATGGC | 36 | |
| | | ATTTCTAG | | |
| Etop-L6d | SEQ ID No. 55 | GTAAGTCTCCAACATCAAGGAAGATGGCA | 36 | |
| | | TTTCTAG | | |
| Etep-L6e | SEQ ID No. 56 | TAAGTACTCCAACATCAAGGAAGATGGCA | 36 | |
| | | TTTCTAG | | |
| Etep-L6f | SEQ ID No. 57 | AAGTATCTCCAACATCAAGGAAGATGGCA | 36 | |
| | | TTTCTAG | | |
| Etep-L7a | SEQ ID No. 58 | CAGGTAACTCCAACATCAAGGAAGATGG | 37 | |
| | | CATTTCTAG | | |
| Etep-L7b | SEQ ID No. 59 | AGGTAAGCTCCAACATCAAGGAAGATGG | 37 | |
| | | CATTTCTAG | | |
| Etep-L7c | SEQ ID No. 60 | GGTAAGTCTCCAACATCAAGGAAGATGGC | 37 | Yes |
| | | ATTTCTAG | | |
| Etep-L7d | SEQ ID No. 61 | GTAAGTACTCCAACATCAAGGAAGATGGC | 37 | |
| | | ATTTCTAG | | |
| Etep-L7e | SEQ ID No. 62 | TAAGTATCTCCAACATCAAGGAAGATGGC | 37 | |
| | | ATTTCTAG | | |
| Etep-L8a | SEQ ID No. 63 | CAGGTAAGCTCCAACATCAAGGAAGATG | 38 | |
| | | GCATTTCTAG | | |
| Etep-L8b | SEQ ID No. 64 | AGGTAAGTCTCCAACATCAAGGAAGATG | 38 | Yes |
| | | GCATTTCTAG | | |
| Etep-L8c | SEQ ID No. 65 | GGTAAGTACTCCAACATCAAGGAAGATG | 38 | Yes |
| | | GCATTTCTAG | | |
| Etep-L8d | SEQ ID No. 66 | GTAAGTATCTCCAACATCAAGGAAGATGG | 38 | |
| | | CATTTCTAG | | |
| Etep-L9a | SEQ ID No. 67 | CAGGTAAGTCTCCAACATCAAGGAAGATG | 39 | |
| | | GCATTTCTAG | | |
| Etep-L9b | SEQ ID No. 68 | AGGTAAGTACTCCAACATCAAGGAAGAT | 39 | Yes |
| | | GGCATTTCTAG | | |
| Etep-L90 | SEQ ID No. 69 | GOTAAGTATCTCCAACATCAAGGAAGATG | 39 | |
| | | GCATTTCTAG | | |
| Etep-L10a | SEQ ID No. 70 | CAGGTAAGTACTCCAACATCAAGGAAGAT | 40 | Yes |
| | | GGCATTTCTAG | | |
| Etep-L10b | SEQ ID No. 71 | AGGTAAGTATCTCCAACATGAAGGAAGAT | 40 | |
| | | GGCATTTCTAG | | |
| Etep-L11 | SEQ ID No. 50 | CAGGTAAGTATCTCCAACATCAAGGAAGA | 41 | |
| | | TGGCATTTCTAG | | |
| Etep-L12 | SEQ ID No. 72 | CCAGGTAAGTATCTCCAACATCAAGGAAG | 42 | |
| | | ATGGCATTTCTAG | | |
| Etep-L13 | SEQ ID No. 73 | GCCAGGTAAGTATCTCCAACATCAAGGAA | 43 | |
| | | GATGGCATTTCTAG | | |

### Example 6: ASO Induced Exon 51 Skipping of DMD Gene with Decoy Sequences of Varying Lengths Attached to the 5′ End (“L”) of Targeting Sequence 000A

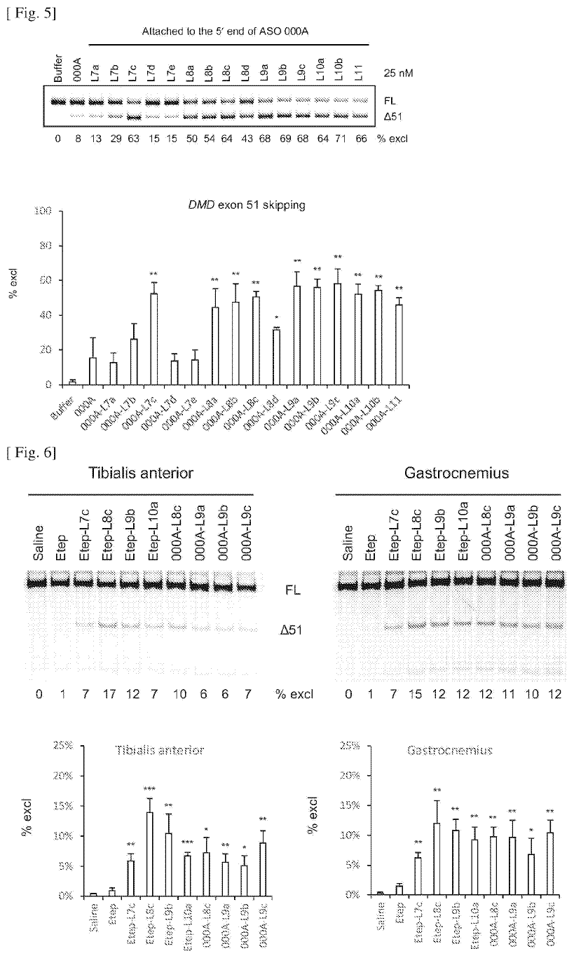

Bipartite ASOs having decoy sequences of varying lengths may have effects of different degrees on the ability of the ASO to promote exon skipping in the DMD gene, for example, as shown in, FIG. 5. 15 decoys with lengths from 7 to 11 nt were tested. The decoy sequences were positioned at the 5′ end of the bipartite ASOs. Targeting sequence 000A is 21-nt long and targets DMD exon 51 from position 50 to position 70. All ASOs tested were MOE-modified with PS backbone and all cytosines were 5-methyl cytosines. Each ASO at 25 nM was transfected into RD cells using Lipofectamine 2000. Buffer alone was used as a negative control. Two days post transfection, cells were collected, and total RNA samples were isolated for splicing analysis by semi-quantitative fluorescent RT-PCR. ASO 000A slightly promoted DMD exon 51 skipping. Bipartite ASOs comprising the decoys at the 5′ end (“L”) improved the exon skipping effect of 000A with statistical significance. One representative gel of three independent experiments is shown in FIG. 5 (top panel, FL: full-length transcript, 451: exon 51-skipped transcript). Quantitation of data (n=3) is shown in the bottom panel of FIG. 5, as mean±standard deviation. *P<0.05, **P<0.01, all vs 000A. 000A-L7c, 000A-L8c, 000A-L9a, 000A-L9b, 000A-L9c, 000A-L10a, and 000A-L10b are optimal ASOs (FIG. 5). See Table 1 for sequence information of decoy sequences and Table 4 for sequence information of ASOs and the targeting sequence tested in Example 6, the targeted sequence (5′ to 3′) in DMD exon 51 is AACUGCCAUCUCCAAACUAGA (SEQ ID No. 74).

TABLE 4
The ASOs and the targeting sequence tested in Example 6

| | | | Length | Optimal ASOs |
|---|---|---|---|---|
| ASO name | SEQ ID No. | Sequence (5′ to 3′) | (nt) | for exon skipping |
| 000A | SEQ ID No. 75 | TCTAGTTTGGAGATGGCAGTT | 21 | |
| 000A-L7a | SEQ ID No. 76 | CAGGTAATCTAGTTTGGAGATGGCAGTT | 28 | |
| 000A-L7b | SEQ ID No. 77 | AGGTAAGTCTAGTTTGGAGATGGCAGTT | 28 | |
| 000A-L7c | SEQ ID No. 78 | GGTAAGTTCTAGTTTGGAGATGGCAGTT | 28 | Yes |
| 000A-L7d | SEQ ID No. 79 | GTAAGTATCTAGTTTGGAGATGGCAGTT | 28 | |
| 000A-L7e | SEQ ID No. 80 | TAAGTATTCTAGTTTGGAGATGGCAGTT | 28 | |
| 000A-L8a | SEQ ID No. 81 | CAGGTAAGTCTAGTTTGGAGATGGCAGT | 29 | |
| | | T | | |
| 000A-L8b | SEQ ID No. 82 | AGGTAAGTTCTAGTTTGGAGATGGCAGT | 29 | |
| | | T | | |
| 000A-L8c | SEQ ID No. 83 | GGTAAGTATCTAGTTTGGAGATGGCAGT | 29 | Yes |
| | | T | | |
| 000A-L8d | SEQ ID No. 84 | GTAAGTATTCTAGTTTGGAGATCGCAGT | 29 | |
| | | T | | |
| 000A-L9a | SEQ ID No. 85 | CAGGTAAGTTCTAGTTTGGAGATGGCAG | 30 | Yes |
| | | TT | | |
| 000A-L9b | SEQ ID No. 86 | AGGTAAGTATCTAGTTTGGAGATGGCA | 30 | Yes |
| | | GTT | | |
| 000A-L9c | SEQ ID No. 87 | GGTAAGTATTCTAGTTTGGAGATGGCAG | 30 | Yes |
| | | TT | | |
| 000A-L10a | SEQ ID No. 88 | CAGGTAAGTATCTAGTTTGGAGATGGCA | 31 | Yes |
| | | GTT | | |
| 000A-L10b | SEQ ID No. 89 | AGGTAAGTATTCTAGTTTGGAGATGGCA | 31 | Yes |
| | | GTT | | |
| 000A-L11 | SEQ ID No. 90 | CAGGTAAGTATTCTAGTTTGGAGATGGC | 32 | |
| | | AGTT | | |

### Example 7: ASO Induced Exon 51 Skipping of DMD Gene in Tibialis Anterior and Gastrocnemius of DMD-Humanized Mice

Several bipartite ASOs were tested in DMD-humanized mice for DMD exon 51 skipping. In the mouse model, the 10766-nt mouse genomic fragment from the last 9053-nt of DMD intron 50, 233-nt exon 51, to the first 1480-nt intron 51 was replaced by corresponding human genomic fragment (800-nt intron 50, 233-nt exon 51 and 800-nt intron 51). Each ASO at 150 mg/kg/injection was administered every other day intravenously into humanized male mice that were ˜8 weeks of age. A total of three injections were given. Three days after the last injection, the mice were euthanized, and muscle samples were collected for RT-PCR analysis by semi-quantitative fluorescent RT-PCR. As shown in FIG. 6, all bipartite ASOs promoted DMD exon 51 skipping with statistical significance compared to MOE/PS version of targeting sequence eteplirsen (Etep) alone. The most potent one, Etep-L8c showed over 10-fold increase in exon 51 skipping. One representative of three independent experiments is shown in FIG. 6 (left upper panel for tibialis anterior and right upper panel for gastrocnemius, FL: full-length transcript, 451: exon 51-skipped transcript). Quantitation of data (n=3) is shown in the bottom panels of FIG. 6 (left bottom panel for tibialis anterior and right bottom panel for gastrocnemius), as mean±standard deviation. *P<0.05, **P<0.01, ***P<0.001, all vs Etep. See Table 1 for sequence information of decoy sequences and Tables 3 and 4 for sequence information of ASOs and the targeting sequence tested in Example 7.

Certain ASOs comprising a decoy increased exon skipping rate to over 14-fold on average compared to ASOs comprising the same targeting sequence but without a decoy.

### Example 8: ASO Induced Exon 53 Skipping of DMD Gene with Decoy Sequences of Varying Lengths

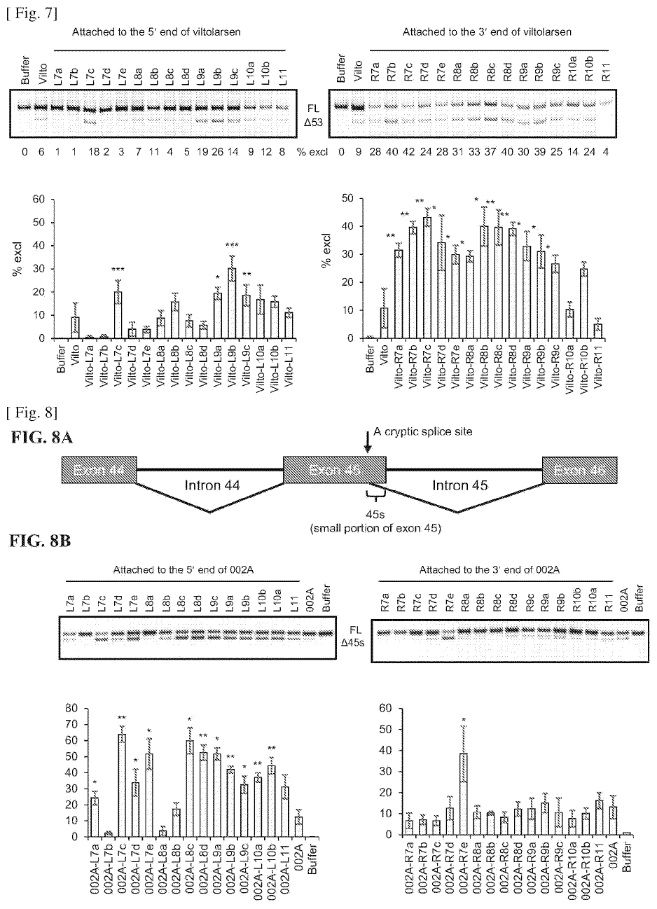

15 decoys with lengths from 7 to 11 nt were tested in the context of the MOE/PS version of targeting sequence viltolarsen (Vilto) in RD cells. Viltolarsen is an FDA approved 21-nt PMO used for treating DMD; it promotes DMD exon 53 skipping through targeting a region in the exon from position 36 to position 56. Each ASO at 25 nM was transfected into RD cells using Lipofectamine 2000. Buffer alone was used as a negative control. Two days post transfection, cells were collected, and total RNA samples were isolated for splicing analysis by semi-quantitative fluorescent RT-PCR. Bipartite ASOs tested comprised a decoy attached to the 5′ (“L”) end of targeting sequence viltolarsen (L7a to L7c, L8a to L8d, L9a to L9c, L10a and L10b, and L11) or the 3′ (“R”) end of targeting sequence viltolarsen (R7a to R7c, R8a to R8d, R9a to R9c, R10a and R10b, and R11). As shown in FIG. 7, targeting sequence viltolarsen alone (Vilto) slightly promoted DMD exon 53 skipping. Some bipartite ASOs improved the exon skipping effect of viltolarsen alone with statistical significance, particularly when the decoys were placed at the 3′ end of the bipartite ASOs. One representative of three independent experiments is shown in FIG. 7 (left upper for bipartite ASOs comprising a decoy attached to the 5′ end of targeting sequence viltolarsen and right upper panel for bipartite ASOs comprising a decoy attached to the 3′ end of targeting sequence viltolarsen, FL: full-length transcript, 453: exon 53-skipped transcript). Quantitation of data (n=3) is shown in the bottom panels of FIG. 7 (left bottom panel for bipartite ASOs comprising a decoy attached to the 5′ end of targeting sequence viltolarsen and right bottom panel for bipartite ASOs comprising a decoy attached to the 3′ end of targeting sequence viltolarsen), as mean±standard deviation. *P<0.05, **P<0.01, ***P<0.001 all vs viltolarsen. ASOs Vilto-L9b, Vilto-R7b, Vilto-R7c, Vilto-R8b, Vilto-R8c, and Vilto-R8d are optimal ASOs (FIG. 7). See Table 1 for sequence information of decoy sequences and Table 5 for sequence information of ASOs and the targeting sequence tested in Example 8, the targeted sequence (5′ to 3′) in DMD exon 53 is GAACACCUUCAGAACCGGAGG (SEQ ID No. 91).

TABLE 5
The ASOs and the targeting sequence tested in Example 8

| | | | | Optimal |
|---|---|---|---|---|
| | | | | ASOs for |
| | | | Length | exon |
| ASO name | SEQ ID No. | Sequence (5′ to 3′) | (nt) | skipping |
| Vilto | SEQ ID No. 92 | CCTCCGGTTCTGAAGGTGTTC | 21 | |
| Vilto-L7a | SFQ ID No. 93 | CAGGTAACCTCCGGTTCTGAAGGTGTTC | 28 | |
| Vilto-L7b | SEQ ID No. 94 | AGGTAAGCCTCCGGTTCTGAAGGTGTTC | 28 | |
| Vilto-L7c | SEQ ID No. 95 | GGIAAGTCCTCCGGTTCTGAAGGTGTTC | 28 | |
| Vilto-L7d | SEQ ID No. 96 | GTAAGTACCTCCGGTTCTGAAGGTGTTC | 28 | |
| Vilto-L7e | SEQ ID No. 91 | TAAGTATCCTCCGGTTCTGAAGGTGTTC | 28 | |
| Vilto-L8a | SEQ ID No. 98 | CAGGTAAGCCTCCGGTTCTGAAGGTGTTC | 29 | |
| Vilto-L8b | SEQ ID No. 99 | AGGTAAGTCCTCCGGTTCTGAAGGTGTTC | 29 | |
| Vilto-L8c | SEQ ID No. 100 | GGTAAGTACCTCCGGTTCTGAAGGTGTTC | 29 | |
| Vilto-L8d | SEQ ID No. 101 | GTAAGTATCCTCCGGTTCTGAAGGTGTTC | 29 | |
| Vilto-19a | SEQ ID No. 102 | CAGGTAAGTCCTCCGGTTCTGAAGGTGTTC | 30 | |
| Vilto-L9b | SEQ ID No. 103 | AGGTAAGTACCTCCGGTTCTGAAGGTGTTC | 30 | Yes |
| Vilto-L9c | SEQ ID No. 104 | GGTAAGTATCCTCCGGTTCTGAAGGTGTTC | 30 | |
| Vilto-L10a | SEQ ID No. 105 | CAGGTAAGTACCTCCGGTTCTGAAGGTGTTC | 31 | |
| Vilto-L10b | SEQ ID No. 106 | AGGTAAGTATCCTCCGGTTCTGAAGGTGTTC | 31 | |
| Vilto-L11 | SEQ ID No. 107 | CAGGTAAGTATCCTCCGGTTCTGAAGGTGTTC | 32 | |
| Vilto-R7a | SEQ ID No. 108 | CCTCCGGTTCTGAAGGTGTTCCAGGTAA | 28 | |
| Vilto-R7b | SEQ ID No. 109 | CCTCCGGTTCTGAAGGTGTTCAGGTAAG | 28 | Yes |
| Vilto-R7C | SEQ ID No. 110 | CCTCCGGTTCTGAAGGTGTTCGGTAAGT | 28 | Yes |
| Vilto-R7d | SEQ ID No. 111 | CCTCCGGTTCTGAAGGTGTTCGTAAGTA | 28 | |
| Vilto-R7e | SEQ ID No. 112 | CCTCCGGTTCTGAAGGTGTTCTAAGTAT | 28 | |
| Vilto-R8a | SEQ ID No. 113 | CCTCCGGTTCTGAAGGTGTTCCAGGTAAG | 29 | |
| Vilto-R8b | SEQ ID No. 114 | CCTCCGGTTCTGAAGGTGTTCAGGTAAGT | 29 | Yes |
| Vilto-R8c | SEQ ID No. 115 | CCTCCGOTTCTGAAGCTCTTCGGTAAGTA | 29 | Yes |
| Vilto-R8d | SEQ ID No. 116 | CCTCCGGTTCTGAAGGTGTTCGTAAGTAT | 29 | Yes |
| Vilto-R9a | SEQ ID No. 117 | CCTCCGGTTCTGAAGGTGTTCCAGGTAAGT | 30 | |
| Vilto-R9b | SEQ ID No. 118 | CCTCCGGTTCTGAAGGTGTTCAGGTAAGTA | 30 | |
| Vilto-R9c | SEQ ID No. 119 | CCTCCGGTTCTGAAGGTGTTCGGTAAGTAT | 30 | |
| Vilto-R10a | SEQ ID No. 120 | CCTCCGGTTCTGAAGGTGTTCCAGGTAAGTA | 31 | |
| Vilto-R10b | SEQ ID No. 121 | CCTCCGGTTCTGAAGGTGTTCAGGTAAGTAT | 31 | |
| Vilto-R11 | SEQ ID No. 122 | CCTCCGGTTCTGAAGGTGTTCCAGGTAAGTAT | 32 | |

### Example 9: ASO Induced Partial Exon 45 Skipping of the DMD Gene with Decoy Sequences of Varying Lengths

15 decoys with lengths from 7 to 11 nt were tested in the context of targeting sequence 002A. ASO 002A targets the junction of DMD exon 45 and intron 45 and promotes skipping of the last 32 nt of exon 45 (here referred to as partial exon skipping), which can restore the reading frame in patients carrying frameshift mutations the same way as by skipping of the whole 176-nt exon 45. As shown in FIG. 8A, ASO 002A may activate a cryptic 5′ splice site in DMD exon 45 (as pointed by an arrow), which leads to skipping of the last 32 nt of the exon. The small 32-nt portion of DMD exon 45 is referred to as 45s. Skipping of 45s instead of the whole 176-nt exon may benefit patients.

Each ASO at 25 nM was transfected into RD cells using Lipofectamine 2000. Buffer alone was used as a negative control. Two days post transfection, cells were collected, and total RNA samples were isolated for splicing analysis by semi-quantitative fluorescent RT-PCR. Bipartite ASOs tested comprised a decoy attached to the 5′ (“L”) end of targeting sequence 002A (L7a to L7e, L8a to L8d, L9a to L9c, L10a and L10b, and L11, FIG. 8B, left column) or the 3′ (“R”) end of targeting sequence 002A (R7a to R7e, R8a to R8d, R9a to R9c, R10a and R10b, and R11, FIG. 8B, right column). Targeting sequence 002A alone (002A) slightly promoted skipping of the 32-nt portion of exon 45 (FIG. 8B). Some bipartite ASOs improved the partial exon skipping effect of targeting sequence 002A alone with statistical significance, particularly when the decoys were placed at the 5′ end of the bipartite ASOs. One representative of three independent experiments is shown in FIG. 8B (left upper panel for bipartite ASOs comprising a decoy attached to the 5′ end of targeting sequence 002A and right upper panel for bipartite ASOs comprising a decoy attached to the 3′ end of targeting sequence 002A, FL: full-length transcript, 445s: 45s-skipped transcript). Quantitation of data (n=3) is shown in the bottom panels of FIG. 8B (left bottom panel for bipartite ASOs comprising a decoy attached to the 5′ end of targeting sequence 002A and right bottom panel for bipartite ASOs comprising a decoy attached to the 3′ end of targeting sequence 002A), as mean±standard deviation. *P<0.05, **P<0.01 all vs 002A. ASOs 002A-L7c, 002A-L8c, and 002A-R7e are optimal ASOs (FIG. 8B). See Table 1 for sequence information of decoy sequences and Table 6 for sequence information of ASOs and the targeting sequence tested in Example 9, the targeted sequence (5′ to 3′) at DMD exon 45 and intron 45 junction is CAGAAAAAAGAGGUAGGGCGAC (SEQ ID No. 123).

TABLE 6
The ASOs and the targeting sequence tested in Example 9

| | | | | Optimal |
|---|---|---|---|---|
| | | | Length | ASOs for |
| ASO name | SEQ ID No. | Sequence (5′ to 3′) | (nt) | exon skipping |
| 002A | SEQ ID No. 124 | GTCGCCCTACCTCTTTTTTCTG | 22 | |
| 002A-L7a | SEQ ID No. 125 | CAGGTAAGTCGCCCTACCTCTTTTTTCTG | 29 | |
| 002A-L7b | SEQ ID No. 126 | AGGTAAGGTCGCCCTACCTCTTTTTTCTG | 29 | |
| 002A-L7c | SEQ ID No. 127 | GGTAAGTGTCGCCCTACCTCTTTTTTCTG | 29 | Yes |
| 002A-L7d | SEQ ID No. 128 | GTAAGTAGTCGCCCTACCTCTTTTTTCTG | 29 | |
| 002A-L7c | SEQ ID No. 129 | TAAGTATGTCGCCCTACCTCTTTTTTCTG | 29 | |
| 002A-L8a | SEQ ID No. 130 | CAGGTAAGGTCGCCCTACCTCTTTTTTCTG | 30 | |
| 002A-L8b | SEQ ID No. 131 | AGGTAAGTGTCGCCCTACCTCTTTTTTCTG | 30 | |
| 002A-L8c | SEQ ID No. 132 | GGTAAGTAGTCGCCCTACCTCTTTTTTCTG | 30 | Yes |
| 002A-L8d | SEQ ID No. 133 | GTAAGTATGTCGCCCTACCTCTTTTTTCTG | 30 | |
| 002A-L9a | SEQ ID No. 134 | CAGGTAAGTGTCGCCCTACCTCTTTTTTCTG | 31 | |
| 002A-L9b | SEQ ID No. 135 | AGGTAAGTAGTCGCCCTACCTCTTTTTTCTG | 31 | |
| 002A-L9c | SEQ ID No. 136 | GGTAAGTATGTCGCCCTACCTCTTTTTTCTG | 31 | |
| 002A-L10a | SEQ ID No. 137 | CAGGTAAGTAGTCGCCCTACCTCTTTTTTCTG | 32 | |
| 002A-L10b | SEQ ID No. 138 | AGGTAAGTATGTCGCCCTACCTCTTTTTTCTG | 32 | |
| 002A-L11 | SEQ ID No. 139 | CAGGTAAGTATGTCGCCCTACCTCTTTTTTCTG | 33 | |
| 002A-R7a | SEQ ID No. 140 | GTCGCCCTACCTCTTTTTTCTGCAGGTAA | 29 | |
| 002A-R7b | SEQ ID No. 141 | GTCGCCCTACCTCTTTTTTCTGAGGTAAG | 29 | |
| 002A-R7c | SEQ ID No. 142 | GTCGCCCTACCTCTTTTTTCTGGGTAAGT | 29 | |
| 002A-R7d | SEQ ID No. 143 | GTCGCCCTACCTCTTTTTTCTGGTAAGTA | 29 | |
| 002A-R7e | SEQ ID No. 144 | GTCGCCCTACCTCTTTTTTCTGTAAGTAT | 29 | Yes |
| 002A-R8a | SEQ ID No. 145 | GTCGCCCTACCTCTTTTTTCTGCAGGTAAG | 30 | |
| 002A-R8b | SEQ ID No. 146 | GTCGCCCTACCTCTTTTTTCTGAGGTAAGT | 30 | |
| 002A-R8c | SEQ ID No. 147 | GTCGCCCTACCTCTTTTTTCTGGGTAAGTA | 30 | |
| 002A-R8d | SEQ ID No. 148 | GTCGCCCTACCTCTTTTTTCTGGTAAGTAT | 30 | |
| 002A-R9a | SEQ ID No. 149 | GTCGCCCTACCTCTTTTTTCTGCAGGTAAGT | 31 | |
| 002A-R9b | SEQ ID No. 150 | GTCGCCCTACCTCTTTTTTCTGAGGTAAGTA | 31 | |
| 002A-R9c | SEQ ID No. 151 | GTCGCCCTACCTCTTTTTTCTGGGTAAGTAT | 31 | |
| 002A-R10a | SEQ ID No. 152 | GTCGCCCTACCTCTTTTTTCTGCAGGTAAGTA | 32 | |
| 002A-R10b | SEQ ID No. 153 | GTCGCCCTACCTCTTTTTTCTGAGGTAAGTAT | 32 | |
| 002A-R11 | SEQ ID No. 154 | GTCGCCCTACCTCTTTTTTCTGCAGGTAAGTAT | 33 | |

### Example 10: ASO-Induced Exon 17 Skipping of APP Gene with Decoy Sequences of Varying Lengths

15 decoy sequences with lengths from 7 to 11 nt were tested in the context of targeting sequence 014B. ASO 014B targets exon 17 of the APP gene and promotes skipping of the exon in the endogenous APP gene. Each ASO at 50 nM was transfected into HEK293T cells using Lipofectamine 2000, and buffer alone was used as a negative control. Two days post transfection, cells were collected, and total RNA samples were isolated for splicing analysis by semi-quantitative fluorescent RT-PCR.

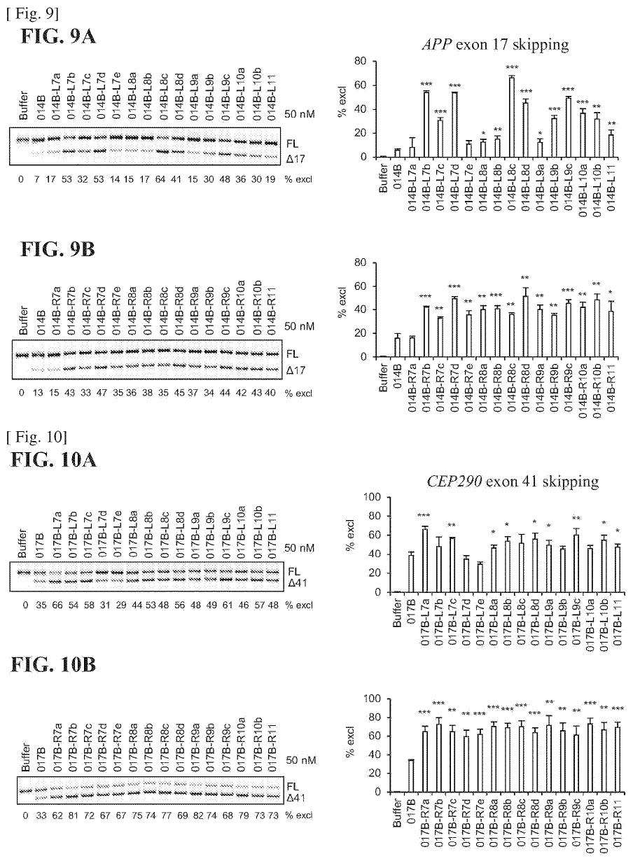

Bipartite ASOs tested comprised a decoy attached to the 5′ (“L”) end of targeting sequence 014B (014B-L7a to 014B-L7e, 014B-L8a to 014B-L8d, 014B-L9a to 014B-L9c, 014B-L10a and 014B-L10b, and 014B-L11, FIG. 9A) or the 3′ (“R”) end of targeting sequence 014B (014B-R7a to 014B-R7e, 014B-R8a to 014B-R8d, 014B-R9a to 014B-R9c, 014B-R10a and 014B-R10b, and 014B-R11, FIG. 9B).

Most bipartite ASOs improved the exon skipping effect of targeting sequence 014B alone (014B) with statistical significance, whether the decoys were attached to the 5′ or the 3′ end of the bipartite ASOs, with the most potent one being 014B-L8c. One representative of three independent experiments for bipartite ASOs comprising a decoy attached to the 5′ end of targeting sequence 014B and bipartite ASOs comprising a decoy attached to the 3′ end of targeting sequence 014B is shown in the left panels of FIGS. 9A and 9B, respectively (FL: full-length transcript, A17: exon 17-skipped transcript). Quantitation of data (n=3) is shown in the right panels of FIGS. 9A and 9B (right panel of FIG. 9A for bipartite ASOs comprising a decoy attached to the 5′ end of targeting sequence 014B and right panel of FIG. 9B for bipartite ASOs comprising a decoy attached to the 3′ end of targeting sequence 014B), as mean±standard deviation. *P<0.05, **P<0.01, ***P<0.001 (all vs 014B). ASO 014B-L8c is an optimal ASO. See Table 1 for sequence information of decoys and Table 7 for sequence information of ASOs and the targeting sequence tested in Example 10, the targeted sequence (5′ to 3′) at the intron 16 and exon 17 junction of APP is UCAAGGUGUUCUUUGCAG (SEQ ID No. 155).

TABLE 7
The ASOs and the targeting sequence tested in Example 10

| | | | Length | Optimal ASOs for |
|---|---|---|---|---|
| ASO name | SEQ ID NO. | Sequence (5′ to 3′) | (nt) | exon skipping |
| 014B | SEQ ID No. 156 | CTGCAAAGAACACCTTGA | 18 | |
| 014B-L7a | SEQ ID No. 157 | CAGGTAACTGCAAAGAACACCTTGA | 25 | |
| 014B-L7b | SEQ ID No. 158 | AGGTAAGCTGCAAAGAACACCTTGA | 25 | |
| 014B-L7c | SEQ ID No. 159 | GGTAAGTCTGCAAAGAACACCTTGA | 25 | |
| 014B-L7d | SEQ ID No. 160 | GTAAGTACTGCAAAGAACACCTTGA | 25 | |
| 014B-L7e | SEQ ID No. 161 | TAAGTATCTGCAAAGAACACCTTGA | 25 | |
| 014B-L8a | SEQ ID No. 162 | CAGGTAAGCTGCAAAGAACACCTTGA | 26 | |
| 014B-L8b | SEQ ID No. 163 | AGGTAAGTCTGCAAAGAACACCTTGA | 26 | |
| 014B-L8c | SEQ ID No. 164 | GGTAAGTACTGCAAAGAACACCTTGA | 26 | Yes |
| 014B-L8d | SEQ ID No. 165 | GTAAGTATCTGCAAAGAACACCTTGA | 26 | |
| 014B-L9a | SEQ ID No. 166 | CAGGTAAGTCTGCAAAGAACACCTTGA | 27 | |
| 014B-L9b | SEQ ID No. 167 | AGGTAAGTACTGCAAAGAACACCTTGA | 27 | |
| 014B-L9c | SEQ ID No. 168 | GGTAAGTATCTGCAAAGAACACCTTGA | 27 | |
| 014B-L10a | SEQ ID No. 169 | CAGGTAAGTACTGCAAAGAACACCTTGA | 28 | |
| 014B-L10b | SEQ ID No. 170 | AGGTAAGTATCTGCAAAGAACACCTTGA | 28 | |
| 014B-L11 | SEQ ID No. 171 | CAGGTAAGTATCTGCAAAGAACACCTTGA | 29 | |
| 014B-R7a | SEQ ID No. 172 | CTGCAAAGAACACCTTGACAGGTAA | 25 | |
| 014B-R7b | SEQ ID No. 173 | CTGCAAAGAACACCTTGAAGGTAAG | 25 | |
| 014B-R7c | SEQ ID No. 174 | CTGCAAAGAACACCTTGAGGTAAGT | 25 | |
| 014B-R7d | SEQ ID No. 175 | CTGCAAAGAACACCTTGAGTAAGTA | 25 | |
| 014B-R7e | SEQ ID No. 176 | CTGCAAAGAACACCTTGATAAGTAT | 25 | |
| 014B-R8a | SEQ ID No. 177 | CTGCAAAGAACACCTTGACAGGTAAG | 26 | |
| 014B-R8b | SEQ ID No. 178 | CTGCAAAGAACACCTTGAAGGTAAGT | 26 | |
| 014B-R8c | SEQ ID No. 179 | CTGCAAAGAACACCTTGAGGTAAGTA | 26 | |
| 014B-R8d | SEQ ID No. 180 | CTGCAAAGAACACCTTGAGTAAGTAT | 26 | |
| 014B-R9a | SEQ ID No. 181 | CTGCAAAGAACACCTTGACAGGTAAGT | 27 | |
| 014B-R9b | SEQ ID No. 182 | CTGCAAAGAACACCTTGAAGGTAAGTA | 27 | |
| 014B-R9c | SEQ ID No. 183 | CTGCAAAGAACACCTTGAGGTAAGTAT | 27 | |
| 014B-R10a | SEQ ID No. 184 | CTGCAAAGAACACCTTGACAGGTAAGTA | 28 | |
| 014B-R10b | SEQ ID No. 185 | CTGCAAAGAACACCTTGAAGGTAAGTAT | 28 | |
| 014B-R11 | SEQ ID No. 186 | CTGCAAAGAACACCTTGACAGGTAAGTAT | 29 | |

Note:
014B sequence is derived from doi: 10.1016/j.ymthe.2018.02.029 with original name as 17-3.

### Example 11: ASO Induced Exon 41 Skipping of CEP290 Gene with Decoy Sequences of Varying Lengths

15 decoy sequences with lengths from 7 to 11 nt were tested in the context of targeting sequence 017B. ASO 017B targets CEP290 exon 41 from position 26 to position 45 and promotes skipping of the exon. Each ASO at 50 nM was transfected into HEK293 cells using Lipofectamine 2000, and buffer alone was used as a negative control. Two days post transfection, cells were collected, and total RNA samples were isolated for splicing analysis by semi-quantitative fluorescent RT-PCR.

Bipartite ASOs tested comprised a decoy attached to the 5′ (“L”) end of targeting sequence 017B (017B-L7a to 017B-L7e, 017B-L8a to 017B-L8d, 017B-L9a to 017B-L9c, 017B-L10a and 017B-L10b, and 017B-L11, FIG. 10A) or the 3′ (“R”) end of targeting sequence 017B (017B-R7a to 017B-R7e, 017B-R8a to 017B-R8d, 017B-R9a to 017B-R9c, 017B-R10a and 017B-R10b, and 017B-R11, FIG. 10B).

Most bipartite ASOs improved the exon skipping effect of targeting sequence 017B alone (017B) with statistical significance, whether the decoys were attached to the 5′ or the 3′ end of the bipartite ASOs, with the most potent one being 017B-R10a. One representative of three independent experiments for bipartite ASOs comprising a decoy attached to the 5′ end of targeting sequence 017B and bipartite ASOs comprising a decoy attached to the 3′ end of targeting sequence 017B is shown in the left panels of FIGS. 10A and 10B, respectively (FL: full-length transcript, A41: exon 41-skipped transcript). Quantitation of data (n=3) is shown in the right panels of FIGS. 10A and 10B (right panel of FIG. 10A for bipartite ASOs comprising a decoy attached to the 5′ end of targeting sequence 017B and right panel of FIG. 10B for bipartite ASOs comprising a decoy attached to the 3′ end of targeting sequence 017B), as mean±standard deviation. *P<0.05, **P<0.01, ***P<0.001 (all vs 017B). ASOs 017B-L7a, 017B-R7b, 017B-R9a, and 017B-R10a are optimal ASOs. See Table 1 for sequence information of decoys and Table 8 for sequence information of ASOs and the targeting sequence tested in Example 11, the targeted sequence (5′ to 3′) in CEP290 exon 41 is AAAGUCUAAUUGAAGAACUC (SEQ ID No. 187).

TABLE 8
The ASOs and the targeting sequence tested in Example 11

| | | | | Optimal ASOs |
|---|---|---|---|---|
| | | | Length | for exon. |
| ASO name | SEQ ID No. | Sequence (5′ to 3′) | (nt) | skipping |
| 017B | SEQ ID No. 188 | GAGTTCTTCAATTAGACTTT | 20 | |
| 017B-L7a | SEQ ID No. 189 | CAGGTAAGAGTTCTTCAATTAGACTTT | 27 | Yes |
| 017B-L7b | SEQ ID No. 190 | AGGTAAGGAGTTCTTCAATTAGACTTT | 27 | |
| 017B-L7c | SEQ ID No. 191 | GGTAAGTGAGTTCTTCAATTAGACTTT | 27 | |
| 017B-L7d | SEQ ID No. 192 | GTAAGTAGAGTTCTTCAATTAGACTTT | 27 | |
| 017B-L7e | SEQ ID No. 193 | TAAGTATGAGTTCTTCAATTAGACTTT | 27 | |
| 017B-L8a | SEQ ID No. 194 | CAGGTAAGGAGTTCTTCAATTAGACTTT | 28 | |
| 017B-L8b | SEQ ID No. 195 | AGGTAAGTGAGTTCTTCAATTAGACTTT | 28 | |
| 017B-L8c | SEQ ID No. 196 | GGTAAGTAGAGTTCTTCAATTAGACTTT | 28 | |
| 017B-L8d | SEQ ID No. 197 | GTAAGTATGAGTTCTTCAATTAGACTTT | 28 | |
| 017B-L9a | SEQ ID No: 198 | CAGGTAAGTGAGTTCTTCAATTAGACTTT | 29 | |
| 017B-L9b | SEQ ID No. 199 | AGGTAAGTAGAGTTCTTCAATTAGACTTT | 29 | |
| 017B-L9c | SEQ ID No, 200 | GGTAAGTATGAGTTCTTCAATTAGACTTT | 29 | |
| 017B-L10a | SEQ ID No. 201 | CAGGTAAGTAGAGTTCTTCAATTAGACTTT | 30 | |
| 017B-L10b | SEQ ID No. 202 | AGGTAAGTATGAGTTCTTCAATTAGACTTT | 30 | |
| 017B-L11 | SEQ ID No. 203 | CAGGTAAGTATGAGTTCTTCAATTAGACTTT | 31 | |
| 017B-R7a | SEQ ID No. 204 | GAGTTCTTCAATTAGACTTTCAGGTAA | 27 | |
| 017B-R7b | SEQ ID No. 205 | GAGTTCTTCAATTAGACTTTAGGTAAG | 27 | Yes |
| 017B-R7c | SEQ ID No. 206 | GAGTTCTTCAATTAGACTTTGGTAAGT | 27 | |
| 017B-R7d | SEQ ID No. 207 | GAGTTCTTCAATTAGACTTTGTAAGTA | 27 | |
| 017B-R7e | SEQ ID No. 208 | GAGTTCTTCAATTAGACTTTTAAGTAT | 27 | |
| 017B-R8a | SEQ ID No. 209 | GAGTTCTTCAATTAGACTTTCAGGTAAG | 28 | |
| 017B-R8b | SEQ ID No. 210 | GAGTTCTTCAATTAGACTTTAGGTAAGT | 28 | |
| 017B-R8c | SEQ ID No. 211 | GAGTTCTTCAATTAGACTTTGGTAAGTA | 28 | |
| 017B-R8d | SEQ ID No. 212 | GAGTTCTTCAATTAGACTTTGTAAGTAT | 28 | |
| 017B-R9a | SEQ ID No. 213 | GAGTTCTTCAATTAGACTTTCAGGTAAGT | 29 | Yes |
| 017B-R9b | SEQ ID No. 214 | GAGTTCTTCAATTAGACTTTAGGTAAGTA | 29 | |
| 017B-R9c | SEQ ID No. 215 | GAGTTCTTCAATTAGACTTTGGTAAGTAT | 29 | |
| 017B-R10a | SEQ ID No. 216 | GAGTTCTTCAATTAGACTTTCAGGTAAGTA | 30 | Yes |
| 017B-R10b | SEQ ID No. 217 | GAGTTCTTCAATTAGACTTTAGGTAAGTAT | 30 | |
| 017B-R11 | SEQ ID No. 218 | GAGTTCTTCAATTAGACTTTCAGGTAAGTAT | 31 | |

Note:
017B was identified from systematic ASO screening.

### Example 12: ASO Induced Exon 19 Skipping of HER2 Gene with Decoy Sequences of Varying Lengths

15 decoy sequences with lengths from 7 to 11 nt were tested in the context of targeting sequence 024B. ASO 024B targets the sequence covering the last 4 nt of intron 18 and first 11 nt of exon 19 of the HER2 (also called ERBB2) gene and moderately promotes skipping of the exon. Each ASO at 50 nM was transfected into HeLa cells using Lipofectamine 2000, and buffer alone was used as a negative control. Two days post transfection, cells were collected, and total RNA samples were isolated for splicing analysis by semi-quantitative fluorescent RT-PCR.

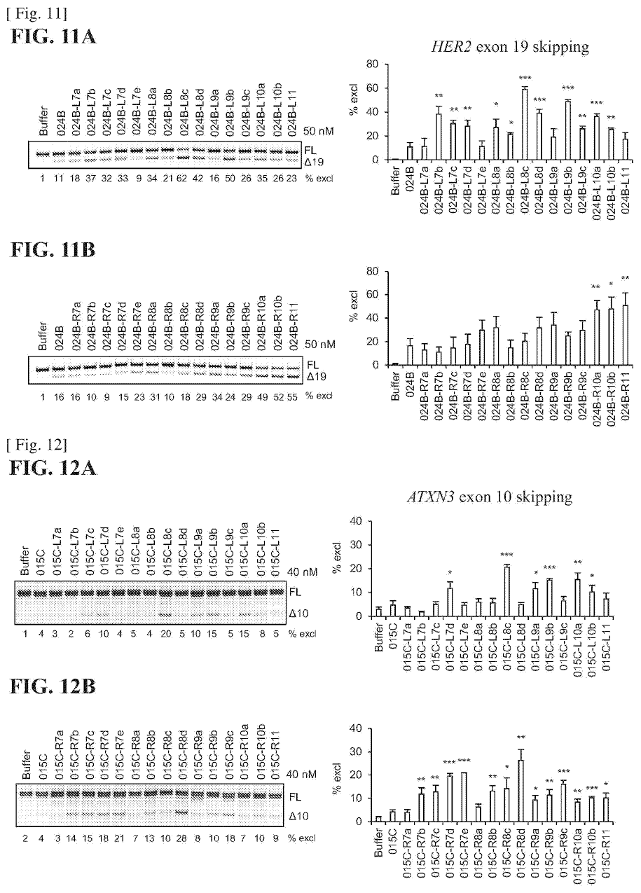

Bipartite ASOs tested comprised a decoy attached to the 5′ (“L”) end of targeting sequence 024B (024B-L7a to 024B-L7e, 024B-L8a to 024B-L8d, 024B-L9a to 024B-L9c, 024B-L10a and 024B-L10b, and 024B-L11, FIG. 11A) or the 3′ (“R”) end of targeting sequence 024B (024B-R7a to 024B-R7e, 024B-R8a to 024B-R8d, 024B-R9a to 024B-R9c, 024B-R10a and 024B-R10b, and 024B-R11, FIG. 11B).

Most bipartite ASOs improved the exon skipping effect of targeting sequence 024B alone (024B) with statistical significance, whether the decoys were attached to the 5′ or the 3′ end of the bipartite ASOs, with the most potent one being 024B-L8c. Representatives of three independent experiments for bipartite ASOs comprising a decoy attached to the 5′ end of targeting sequence 024B and bipartite ASOs comprising a decoy attached to the 3′ end of targeting sequence 024B are shown in the left panels of FIGS. 11A and 11B, respectively (FL: full-length transcript, A19: exon 19-skipped transcript). Quantitation of data (n=3) is shown in the right panels of FIGS. 11A and 11B (right panel of FIG. 11A for bipartite ASOs comprising a decoy attached to the 5′ end of targeting sequence 024B and right panel of FIG. 11B for bipartite ASOs comprising a decoy attached to the 3′ end of targeting sequence 024B), as mean±standard deviation. *P<0.05, **P<0.01, ***P<0.001 (all vs 024B). ASO 024B-L8c is an optimal ASO. See Table 1 for sequence information of decoys and Table 9 for sequence information of ASOs and the targeting sequence tested in Example 12, the targeted sequence (5′ to 3′) at the intron 18 and exon 19 junction of HER2 is CCAGGGCAUCUGGAU (SEQ ID No. 219).

TABLE 9
The ASOs and the targeting sequence tested in Example 12

| | | | | Optimal ASOs |
|---|---|---|---|---|
| | | | Length | for exon |
| ASO name | SEQ ID No. | Sequence (5′ to 3′) | (nt) | skipping |
| 024B | SEQ ID No. 220 | ATCCAGATGCCCTGG | 15 | |
| 024B-L7a | SEQ ID No. 221 | CAGGTAAATCCAGATGCCCTGG | 22 | |
| 024B-L7b | SEQ ID No. 222 | AGGTAAGATCCAGATGCCCTGG | 22 | |
| 024B-L7c | SEQ ID No. 223 | GGTAAGTATCCAGATGCCCTGG | 22 | |
| 024B-L7d | SEQ ID No. 224 | GTAAGTAATCCAGATGCCCTGG | 22 | |
| 024B-L7e | SEQ ID No. 225 | TAAGTATATCCAGATGCCCTGG | 22 | |
| 024B-L8a | SEQ ID No. 226 | CAGGTAAGATCCAGATGCCCTGG | 23 | |
| 024B-L8b | SEQ ID No. 227 | AGGTAAGTATCCAGATGCCCTGG | 23 | |
| 024B-L8c | SEQ ID No. 228 | GGTAAGTAATCCAGATGCCCTGG | 23 | Yes |
| 024B-L8d | SEQ ID No. 229 | GTAAGTATATCCAGATGCCCTGG | 23 | |
| 024B-L9a | SEQ ID No. 230 | CAGGTAAGTATCCAGATGCCCTGG | 24 | |
| 024B-L9b | SEQ ID No. 231 | AGGTAAGTAATCCAGATGCCCTGG | 24 | |
| 024B-L9c | SEQ ID No. 232 | GGTAAGTATATCCAGATGCCCTGG | 24 | |
| 024B-L10a | SEQ ID No. 233 | CAGGTAAGTAATCCAGATGCCCTGG | 25 | |
| 024B-L10b | SEQ ID No. 234 | AGGTAAGTATATCCAGATGCCCTGG | 25 | |
| 024B-L11 | SEQ ID No. 235 | CAGGTAAGTATATCCAGATGCCCTGG | 26 | |
| 024B-R7a | SEQ ID No. 236 | ATCCAGATGCCCTGGCAGGTAA | 22 | |
| 024B-R7b | SEQ ID No. 237 | ATCCAGATGCCCTGGAGGTAAG | 22 | |
| 024B-R7c | SEQ ID No. 238 | ATCCAGATGCCCTGGGGTAAGT | 22 | |
| 024B-R7d | SEQ ID No. 239 | ATCCAGATGCCCTGGGTAAGTA | 22 | |
| 024B-R7e | SEQ ID No. 240 | ATCCAGATGCCCTGGTAAGTAT | 22 | |
| 024B-R8a | SEQ ID No. 241 | ATCCAGATGCCCTGGCAGGTAAG | 23 | |
| 024B-R8b | SEQ ID No. 242 | ATCCAGATGCCCTGGAGGTAAGT | 23 | |
| 024B-R8c | SEQ ID No. 243 | ATCCAGATGCCCTGGGGTAAGTA | 23 | |
| 024B-R8d | SEQ ID No. 244 | ATCCAGATGCCCTGGGTAAGTAT | 23 | |
| 024B-R9a | SEQ ID No. 245 | ATCCAGATGCCCTGGCAGGTAAGT | 24 | |
| 024B-R9b | SEQ ID No. 246 | ATCCAGATGCCCTGGAGGTAAGTA | 24 | |
| 024B-R9c | SEQ ID No. 247 | ATCCAGATGCCCTGGGGTAAGTAT | 24 | |
| 024B-R10a | SEQ ID No. 248 | ATCCAGATGCCCTGGCAGGTAAGTA | 25 | |
| 024B-R10b | SEQ ID No. 249 | ATCCAGATGCCCTGGAGGTAAGTAT | 25 | |
| 024B-R11 | SEQ ID No. 250 | ATCCAGATGCCCTGGCAGGTAAGTAT | 26 | |

Note:
024B sequence is derived from doi: 10.3892/ijo_00000472 with the original name as 2707.

### Example 13: ASO Induced Exon 10 Skipping of ATXN3 Gene with Decoy Sequences of Varying Lengths

15 decoy sequences with lengths from 7 to 11 nt were tested in the context of targeting sequence 015C (with the same sequence as VO659 but different chemical modifications). ASO 015C constitutes 7 copies of CTG and targets seven CAG repeats, and thus can target CAG repeats in exon 10 of the ATXN3 (also called SCA3) gene. ASO 015C promotes exon 10 skipping of the gene. Each ASO at 40 nM was transfected into A549 cells using Lipofectamine 2000, and buffer alone was used as a negative control. Two days post transfection, cells were collected, and total RNA samples were isolated for splicing analysis by semi-quantitative fluorescent RT-PCR.

Bipartite ASOs tested comprised a decoy attached to the 5′ (“L”) end of targeting sequence 015C (015C-L7a to 015C-L7e, 015C-L8a to 015C-L8d, 015C-L9a to 015C-L9c, 015C-L10a and 015C-L10b, and 015C-L11, FIG. 12A) or the 3′ (“R”) end of targeting sequence 015C (015C-R7a to 015C-R7e, 015C-R8a to 015C-R8d, 015C-R9a to 015C-R9c, 015C-R10a and 015C-R10b, and 015C-R11, FIG. 12B).

Most bipartite ASOs improved the exon skipping effect of targeting sequence 015C alone (015C) with statistical significance, whether the decoys were attached to the 5′ or the 3′ end of the bipartite ASOs, with the most potent one being 015C-R8d. Representatives of three independent experiments for bipartite ASOs comprising a decoy attached to the 5′ end of targeting sequence 015C and bipartite ASOs comprising a decoy attached to the 3′ end of targeting sequence 015C are shown in the left panels of FIGS. 12A and 12B, respectively (FL: full-length transcript, A10: exon 10-skipped transcript). Quantitation of data (n=3) is shown in the right panels of FIGS. 12A and 12B (right panel of FIG. 12A for bipartite ASOs comprising a decoy attached to the 5′ end of targeting sequence 015C and right panel of FIG. 12B for bipartite ASOs comprising a decoy attached to the 3′ end of targeting sequence 015C), as mean±standard deviation. *P<0.05, **P<0.01, ***P<0.001 (all vs 015C). ASOs 015C-L8c and 015C-R8d are optimal ASOs. See Table 1 for sequence information of decoys and Table 10 for sequence information of ASOs and the targeting sequence tested in Example 13, the targeted sequence (5′ to 3′) in ATXN3 exon 10 is CAGCAGCAGCAGCAGCAGCAG (SEQ ID No. 251).

TABLE 10
The ASOs and the targeting sequence tested in Example 13

| | | | | Optimal |
|---|---|---|---|---|
| | | | | ASOs for |
| | | | Length | exon |
| ASO name | SEQ ID No. | Sequence (5′ to 3′) | (nt) | skipping |
| 015C | SEQ ID No. 252 | CTGCTGCTGCTGCTGCTGCTG | 21 | |
| 015C-L7a | SEQ ID No. 253 | CAGGTAACTGCTGCTGCTGCTGCTGCTG | 28 | |
| 015C-L7b | SEQ ID No. 254 | AGGTAAGCTGCTGCTGCTGCTGCTGCTG | 28 | |
| 015C-L7c | SEQ ID No. 255 | GGTAAGTCTGCTGCTGCTGCTGCTGCTG | 28 | |
| 015C-L7d | SEQ ID No. 256 | GTAAGTACTGCTGCTGCTGCTGCTGCTG | 28 | |
| 015C-L7c | SEQ ID No. 257 | TAAGTATCTGCTGCTGCTGCTGCTGCTG | 28 | |
| 015C-L8a | SEQ ID No. 258 | CAGGTAAGCTGCTGCTGCTGCTGCTGCTG | 29 | |
| 015C-L8b | SEQ ID No. 259 | AGGTAAGTCTGCTGCTGCTGCTGCTGCTG | 29 | |
| 015C-L8c | SEQ ID No. 260 | GOTAAGTACTGCTGCTGCTGCTGCTGCTG | 29 | Yes |
| 015C-L8d | SEQ ID No. 261 | GTAAGTATCTGCTGCTGCTGCTGCTGCTG | 29 | |
| 015C-L9a | SEQ ID No. 262 | CAGGTAAGTCTGCTGCTGCTGCTGCTGCTG | 30 | |
| 015C-L9b | SEQ ID No. 263 | AGGTAAGTACTGCTGCTGCTGCTGCTGCTG | 30 | |
| 015C-L9c | SEQ ID No. 264 | GGTAAGTATCTGCTGCTGCTGCTGCTGCTG | 30 | |
| 015C-L10a | SEQ ID No. 265 | CAGGTAAGTACTGCTGCTGCTGCTGCTGCTG | 31 | |
| 015C-L10b | SEQ ID No. 266 | AGGTAAGTATCTGCTGCTGCTGCTGCTGCTG | 31 | |
| 015C-L11 | SEQ ID No. 267 | CAGGTAAGTATCTGCTGCTGCTGCTGCTGCTG | 32 | |
| 015C-R7a | SEQ ID No. 268 | CTGCTGCTGCTGCTGCTGCTGCAGGTAA | 28 | |
| 015C-R7b | SEQ ID No. 269 | CTGCTGCTGCTGCTGCTGCTGAGGTAAG | 28 | |
| 015C-R7c | SEQ ID No. 270 | CTGCTGCTGCTGCTGCTGCTGGGTAAGT | 28 | |
| 015C-R7d | SEQ ID No. 271 | CTGCTGCTGCTGCTGCTGCTGGTAAGTA | 28 | |
| 015C-R7c | SEQ ID No. 272 | CTGCTGCTGCTGCTGCTGCTGTAAGTAT | 28 | |
| 015C-R8a | SEQ ID No. 273 | CTGCTGCTGCTGCTGCTGCTGCAGGTAAG | 29 | |
| 015C-R8b | SEQ ID No. 274 | CTGCTGCTGCTGCTGCTGCTGAGGTAAGT | 29 | |
| 015C-R8c | SEQ ID No. 275 | CTGCTGCTGCTGCTGCTGCTGGGTAAGTA | 29 | |
| 015C-R8d | SEQ ID No. 276 | CTGCTGCTGCTGCTGCTGCTGGTAAGTAT | 29 | Yes |
| 015C-R9a | SEQ ID No. 277 | CTGCTGCTGCTGCTGCTGCTGCAGGTAAGT | 30 | |
| 015C-R9b | SEQ ID No. 278 | CTGCTGCTGCTGCTGCTGCTGAGGTAAGTA | 30 | |
| 015C-R96 | SEQ ID No. 279 | CTGCTGCTGCTGCTGCTGCTGGGTAAGTAT | 30 | |
| 015C-R10a | SEQ ID No. 280 | CTGCTGCTGCTGCTGCTGCTGCAGGTAAGTA | 31 | |
| 015C-R10b | SEQ ID No. 281 | CTGCTGCTGCTGCTGCTGCTGAGGTAAGTAT | 31 | |
| 015C-R11 | SEQ ID No. 282 | CTGCTGCTGCTGCTGCTGCTGCAGGTAAGTAT | 32 | |

Note:
015C sequence is derived from doi: 10.1016/j.omtn.2019.07.004 with its original name as
VO659.

### Example 14: ASO Induced Exon 10 Skipping of PKM Gene with Decoy Sequences of Varying Lengths

15 decoy sequences with lengths from 7 to 11 nt were tested in the context of targeting sequence 027B. ASO 027B targets PKM exon 10 from position 45 to position 62 and promote exon 10 skipping and exon 9 inclusion of the endogenous PKM gene. Each ASO at 50 nM was transfected into RD cells using Lipofectamine 2000, and buffer alone was used as a negative control. Two days post transfection, cells were collected, and total RNA samples were isolated for splicing analysis by semi-quantitative fluorescent RT-PCR. The PCR products were digested with PstI to distinguish their origin (PKM1 or PKM2).

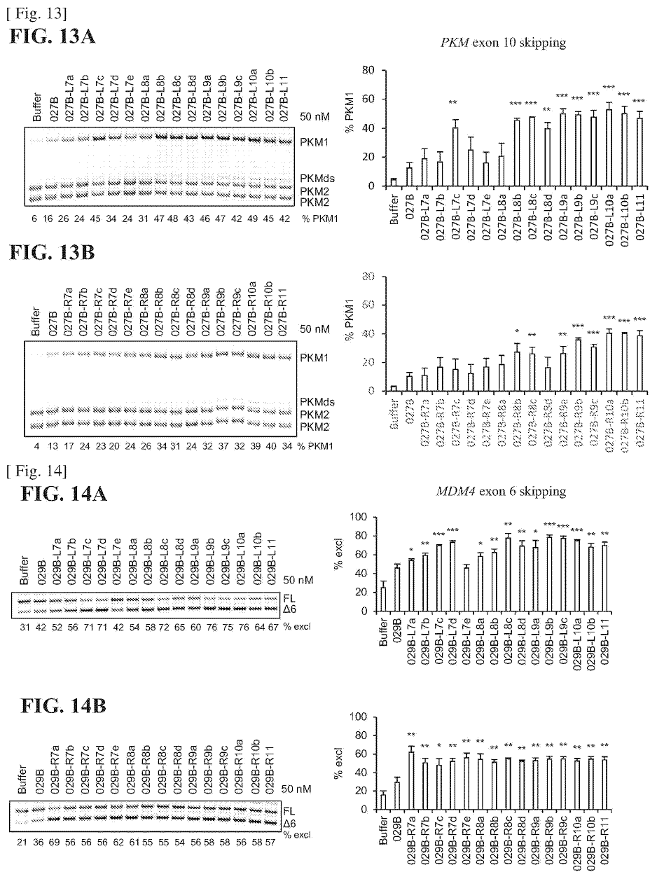

Bipartite ASOs tested comprised a decoy attached to the 5′ (“L”) end of targeting sequence 027B (027B-L7a to 027B-L7e, 027B-L8a to 027B-L8d, 027B-L9a to 027B-L9c, 027B-L10a and 027B-L10b, and 027B-L11, FIG. 13A) or the 3′ (“R”) end of targeting sequence 027B (027B-R7a to 027B-R7e, 027B-R8a to 027B-R8d, 027B-R9a to 027B-R9c, 027B-R10a and 027B-R10b, and 027B-R11, FIG. 13B).

Most bipartite ASOs improved the exon 10 skipping effect of targeting sequence 027B alone (027B) with statistical significance, whether the decoys were attached to the 5′ or the 3′ end of the bipartite ASOs. Representatives of three independent experiments for bipartite ASOs comprising a decoy attached to the 5′ end of targeting sequence 027B and bipartite ASOs comprising a decoy attached to the 3′ end of targeting sequence 027B are shown in the left panels of FIGS. 13A and 13B, respectively (PKM1: exon 10-skipped transcript; PKM2: exon 9-skipped transcript; PKMds: both exons being skipped). Quantitation of data (n=3) is shown in the right panels of FIGS. 13A and 13B (right panel of FIG. 13A for bipartite ASOs comprising a decoy attached to the 5′ end of targeting sequence 027B and right panel of FIG. 13B for bipartite ASOs comprising a decoy attached to the 3′ end of targeting sequence 027B), as mean±standard deviation. *P<0.05, **P<0.01, ***P<0.001 (all vs 027B). ASOs 027B-L8c, 027B-L9a, 027B-L10a, 027B-L10b, and 027B-R10a are optimal ASOs. See Table 1 for sequence information of decoys and Table 11 for sequence information of ASOs and the targeting sequence tested in Example 14, the targeted sequence (5′ to 3′) in PKM exon 10 is UGAGGAACUCCGCCGCCU (SEQ ID No. 283).

TABLE 11
The ASOs and the targeting sequence tested in Example 14

| ASO | | | Length | Optimal ASOs for exon |
|---|---|---|---|---|
| name | SEQ ID No. | Sequence (5′ to 3′) | (nt) | skipping |
| 027B | SEQ ID No. | AGGCGGCGGAGTTCCTCA | 18 | |
| | 284 | | | |
| 027B-L7a | SEQ ID No. | CAGGTAAAGGCGGCGGAGTTCCTCA | 25 | |
| | 285 | | | |
| 027B-L7b | SEQ ID No. | AGGTAAGAGGCGGCGGAGTTCCTCA | 25 | |
| | 286 | | | |
| 027B-L7c | SEQ ID No. | GGTAAGTAGGCGGCGGAGTTCCTCA | 25 | |
| | 287 | | | |
| 027B-L7d | SEQ ID No. | GTAAGTAAGGCGGCGGAGTTCCTCA | 25 | |
| | 288 | | | |
| 027B-L7e | SEQ ID No. | TAAGTATAGGGGGOGGAGTTCCTCA | 25 | |
| | 289 | | | |
| 027B-L8a | SEQ ID No. | CAGGTAAGAGGCGGCGGAGTTCCTCA | 26 | |
| | 290 | | | |
| 027B-L8b | SEQ ID No. | AGGTAAGTAGGCGGCGGAGTTCCTCA | 26 | |
| | 291 | | | |
| 027B-L8c | SEQ ID No. | GGTAAGTAAGGCGGCGGAGTTCCTCA | 26 | Yes |
| | 292 | | | |
| 027B-L8d | SEQ ID No. | GTAAGTATAGGCGGCGGAGTTCCTCA | 26 | |
| | 293 | | | |
| 027B-L9a | SEQ ID No. | CAGGTAAGTAGGCGGCGGAGTTCCTCA | 27 | Yos |
| | 294 | | | |
| 027B-L9b | SEQ ID No. | AGGTAAGTAAGGCGGCGGAGTTCCTCA | 27 | |
| | 295 | | | |
| 027B-L9c | SEQ ID No. | GGTAAGTATAGGCGGCGGAGTTCCTCA | 27 | |
| | 296 | | | |
| 027B-L10a | SEQ ID No. | CAGGTAAGTAAGGCGGCGGAGTTCCTCA | 28 | Yes |
| | 297 | | | |
| 027B-L10b | SEQ ID No. | AGGTAAGTATAGGOGGCGGAGTTCCTCA | 28 | Yes |
| | 1298 | | | |
| 027B-L11 | SEQ ID No. | CAGGTAAGTATAGGCGGCGGAGTTCCTCA | 29 | |
| | 299 | | | |
| 027B-R7a | SEQ ID No. | AGGCGGCGGAGTTCCTCACAGGTAA | 25 | |
| | 300 | | | |
| 027B-R7b | SEQ ID No. | AGGCGGCGGAGTTCCTCAAGGTAAG | 25 | |
| | 301 | | | |
| 027B-R7c | SEQ ID No. | AGGOGGCGGAGTTCCTCAGGTAAGT | 25 | |
| | 302 | | | |
| 027B-R7d | SEQ ID No. | AGGCGGCGGAGTTCCTCAGTAAGTA | 25 | |
| | 303 | | | |
| 027B-R7e | SEQ ID No. | AGGCGGCGGAGTTCCTCATAAGTAT | 25 | |
| | 304 | | | |
| 027B-R8a | SEQ ID No. | AGGCGGCGGAGTTCCTCACAGGTAAG | 26 | |
| | 305 | | | |
| 027B-R8b | SEQ ID No. | AGGCGGCGGAGTTCCTCAAGGTAAGT | 26 | |
| | 306 | | | |
| 027B-R8c | SEQ ID No. | AGGCGGCGGAGTTCCTCAGGTAAGTA | 26 | |
| | 307 | | | |
| 027B-R8d | SEQ ID No. | AGGCGGCGGAGTTCCTCAGTAAGTAT | 26 | |
| | 308 | | | |
| 027B-R9a | SEQ ID No. | AGGCGGCGGAGTTCCTCACAGGTAAGT | 27 | |
| | 309 | | | |
| 027B-R9b | SEQ ID No. | AGGCGGCGGAGTTCCTCAAGGTAAGTA | 27 | |
| | 310 | | | |
| 027B-R9c | SEQ ID No. | AGGCGGCGGAGTTCCTCAGGTAAGTAT | 27 | |
| | 311 | | | |
| 027B-R10a | SEQ ID No. | AGGCGGCGGAGTTCCTCACAGGTAAGTA | 28 | Yes |
| | 312 | | | |
| 027B-R10b | SEQ ID No. | AGGCGGCGGAGTTCCTCAAGGTAAGTAT | 28 | |
| | 313 | | | |
| 027B-R11 | SEQ ID No. | AGGCGGCGGAGTTCCTCACAGGTAAGTAT | 29 | |
| | 314 | | | |

Note:
027B sequence is derived from doi: 10.1158/0008-5472. CAN-20-0948 with its original name as
ASO1-cEt/DNA.

### Example 15: ASO Induced Exon 6 Skipping of MDM4 Gene with Decoy Sequences of Varying Lengths

15 decoy sequences with lengths from 7 to 11 nt were tested in the context of targeting sequence 029B. ASO 029B targets the region covering the last 9 nt of exon 6 and first 16 nt of intron 6 of the MDM4 gene. ASO 029B alone moderately promotes exon 6 skipping of the endogenous MDM4 gene. Each ASO at 50 nM was transfected into HEK293 cells using Lipofectamine 2000, and buffer was used as a negative control. Two days post transfection, cells were collected, and total RNA samples were isolated for splicing analysis by semi-quantitative fluorescent RT-PCR.

Bipartite ASOs tested comprised a decoy attached to the 5′ (“L”) end of targeting sequence 029B (029B-L7a to 029B-L7e, 029B-L8a to 029B-L8d, 029B-L9a to 029B-L9c, 029B-L10a and 029B-L10b, and 029B-L11, FIG. 14A) or the 3′ (“R”) end of targeting sequence 029B (029B-R7a to 029B-R7c, 029B-R8a to 029B-R8d, 029B-R9a to 029B-R9c, 029B-R10a and 029B-R10b, and 029B-R11, FIG. 14B).

All bipartite ASOs except one improved the exon 6-skipping effect of targeting sequence 029B alone (029B) with statistical significance, whether the decoys were attached to the 5′ or the 3′ end of the bipartite ASOs. Representatives of three independent experiments for bipartite ASOs comprising a decoy attached to the 5′ end of targeting sequence 029B and bipartite ASOs comprising a decoy attached to the 3′ end of targeting sequence 029B are shown in the left panels of FIGS. 14A and 14B, respectively (FL: full-length transcript, A6: exon 6-skipped transcript). Quantitation of data (n=3) is shown in the right panels of FIGS. 14A and 14B (right panel of FIG. 14A for bipartite ASOs comprising a decoy attached to the 5′ end of targeting sequence 029B and right panel of FIG. 14B for bipartite ASOs comprising a decoy attached to the 3′ end of targeting sequence 029B), as mean±standard deviation. *P<0.05, **P<0.01, ***P<0.001 (all vs 029B). ASOs 029B-L7d, 029B-L8c, 029B-L9b, 029B-L9c, and 029B-R7a are optimal ASOs. See Table 1 for sequence information of decoys and Table 12 for sequence information of ASOs and the targeting sequence tested in Example 15, the targeted sequence (5′ to 3′) at MDM4 exon 6-intron 6 junction is CAACUGAAGGUAAAAUCACCACACG (SEQ ID No. 315).

TABLE 12
The ASOs and the targeting sequence tested in Example 15

| ASO | SEQ ID | | Length | Optimal ASOs for |
|---|---|---|---|---|
| name | No. | Sequence (5′ to 3′) | (nt) | exon skipping |
| 029B | SEQ ID | CGTGTGGTGATTTTACCTTCAGTTG | 25 | |
| | No. 316 | | | |
| 029B- | SEQ ID | CAGGTAACGTGTGGTGATTTTACCTTCAGTTG | 32 | |
| L7a | No. 317 | | | |
| 029B- | SEQ ID | AGGTAAGCGTGTGGTGATTTTACCTTCAGTTG | 32 | |
| L7b | No. 318 | | | |
| 029B- | SEQ ID | GGTAAGTCGTGTGGTGATTTTACCTTCAGTTG | 32 | |
| L7c | No. 3.19 | | | |
| 029B- | SEQ ID | GTAAGTACGTGTGGTGATTTTACCTTCAGTTG | 32 | Yes |
| L7d | No. 320 | | | |
| 029B- | SEQ ID | TAAGTATCGTGTGGTGATTTTACCTTCAGTTG | 32 | |
| L7e | No, 321 | | | |
| 029B- | SEQ JD | CAGGTAAGCGTGTGGTGATTTTACCTTCAGTTG | 33 | |
| L8a | No. 322 | | | |
| 029B- | SEQ ID | AGGTAAGTCGTGTGGTGATTTTACCTTCAGTTG | 33 | |
| L8b | No. 323 | | | |
| 029B- | SEQ ID | GGTAAGTACGTGTGGTGATTTTACCTTCAGTTG | 33 | Yes |
| L8c | No. 324 | | | |
| 029B- | SEQ ID | GTAAGTATCGTGTGGTGATTTTACCTTCAGTTG | 33 | |
| L8d | No. 325 | | | |
| 029B- | SEQ ID | CAGGTAAGTCGTGTGGTGATTTTACCTTCAGTTG | 34 | |
| L9a | No. 326 | | | |
| 029B- | SEQ ID | AGGTAAGTACGTGTGGTGATTTTACCTTCAGTTG | 34 | Yes |
| L9b | No. 327 | | | |
| 029B- | SEQ ID | GGTAAGTATCGTGTGGTGATTTTACCTTCAGTTG | 34 | Yes |
| L9c | No. 328 | | | |
| 029B- | SEQ ID | CAGGTAAGTACGTGTGGTGATTTTACCTTCAGTTG | 35 | |
| L10a | No. 329 | | | |
| 029B- | SEQ ID | AGGTAAGTATCGTGTGGTGATTTTACCTTCAGTTG | 35 | |
| L10b | No. 330 | | | |
| 029B- | SEQ ID | CAGGTAAGTATCGTGTGGTGATTTTACCTTCAGTTG | 36 | |
| L11 | No. 331 | | | |
| 029B- | SEQ ID | CGTGTGGTGATTTTACCTTCAGTTGCAGGTAA | 32 | Yes |
| R7a | No. 332 | | | |
| 029B- | SEQ ID | CGTGTGGTGATTTTACCTTCAGTTGAGGTAAG | 32 | |
| RTb | No. 333 | | | |
| 029B- | SEQ ID | CGTGTGGTGATTTTACCTTCAGTTGGGTAAGT | 32 | |
| R7c | No. 334 | | | |
| 029B- | SEQ ID | CGTGTGGTGATTTTACCTTCAGTTGGTAAGTA | 32 | |
| R7d | No. 335 | | | |
| 029B- | SEQ ID | CGTGTGGTGATTTTACCTTCAGTTGTAAGTAT | 32 | |
| R7e | No. 336 | | | |
| 029B- | SEQ ID | CGTGTGGTGATTTTACCTTCAGTTGCAGGTAAG | 33 | |
| R8a | No. 337 | | | |
| 029B- | SEQ ID | CGTGTGGTGATTTTACCTTCAGTTGAGGTAAGT | 33 | |
| R8b | No. 338 | | | |
| 029B- | SEQ ID | CGTGTGGTGATTTTACCTTCAGTTGGGTAAGTA | 33 | |
| R&c | No. 339 | | | |
| 029B- | SEQ ID | CGTGTGGTGATTTTACCTTCAGTTGGTAAGTAT | 33 | |
| R&d | No. 340 | | | |
| 029B- | SEQ ID | CGTGTGGTGATTTTACCTTCAGTTGCAGGTAAGT | 34 | |
| R9a | No. 341 | | | |
| 029B- | SEQ ID | CGTGTGGTGATTTTACCTTCAGTTGAGGTAAGTA | 34 | |
| R9b | No. 342 | | | |
| 029B- | SEQ ID | CGTGTGGTGATTTTACCTTCAGTTGOGTAAGTAT | 34 | |
| R9c | No. 343 | | | |
| 029B- | SEQ ID | CGTGTGGTGATTTTACCTTCAGTTGCAGGTAAGTA | 35 | |
| R10a | No. 344 | | | |
| 029B- | SEQ ID | CGTGTGGTGATTTTACCTTCAGTTGAGGTAAGTAT | 35 | |
| R10b | No. 345 | | | |
| 029B- | SEQ ID | CGTGTGGTGATTTTACCTTCAGTTGCAGGTAAGTAT | 36 | |
| R11 | No. 346 | | | |

Note:
029B sequence is derived from doi: 10.1172/JCI82534 with the original name as ASO4.

1. An oligonucleotide comprising or consisting of a targeting sequence, and a 5′-splice-site decoy sequence operably connected at the 5′ end and/or 3′ end of the targeting sequence:
the decoy sequence comprising a nucleotide sequence of 5, 6, 7, 8, 9, 10, or 11 nucleotides that is complementary to the single-strand 5′ end of U1 snRNA;
the targeting sequence hybridizes to a sequence selected from the group consisting of an exon of interest, a flanking intron sequence upstream of the exon of interest, a flanking intron sequence downstream of the exon of interest, an intron-exon junction upstream of the exon of interest, and an intron-exon junction downstream of the exon of interest; and
the decoy sequence operably connected to the 5′ end of the targeting sequence and the decoy sequence operably connected to the 3′ end of the targeting sequence being the same or different.

2. The oligonucleotide of claim 1, the decoy sequence(s) comprising or consisting of one or more nucleotide sequences independently selected from the group consisting of the nucleotide sequences of SEQ ID Nos. 1 to 23 and 347 to 353.

3. The oligonucleotide of any one of the prior claims, the exon of interest being selected from the group consisting of exon 7 of the endogenous SMN1 and SMN2 genes, exons 45, 51 and 52 of the endogenous DMD gene, exon 17 of the endogenous APP gene, exon 41 of the endogenous CEP290 gene, exon 19 of the endogenous HER2 gene, exon 10 of the SCA3 gene, exon 10 of the endogenous PKM gene, and exon 6 of the endogenous MDM4 gene.

4. The oligonucleotide of any one of the prior claims, the targeting sequence comprising or consisting of a nucleotide sequence selected from the group consisting of the nucleotide sequences of SEQ ID Nos. 25, 29, 33, 37, 41, 45, 49, 75, 92, 124, 156, 188, 220, 252, 284, and 316.

5. The oligonucleotide of any one of the prior claims, the targeting sequence being linked to the decoy sequence directly.

6. The oligonucleotide of any one of claims 1 to 4, the targeting sequence being linked to the decoy sequence via a linker having 1, 2, 3, 4, or 5 nucleotides.

7. The oligonucleotide of any one of claims 1 to 6, when a decoy sequence is operably connected at the 5′ end and 3′ end of the targeting sequence, the linker between the targeting sequence and the decoy sequence at the 5′ end of the targeting sequence and the linker between the targeting sequence and the decoy sequence at the 3′ end of the targeting sequence can be the same or different.

8. The oligonucleotide of claim 1 comprising or consisting of a nucleotide sequence selected from the group consisting of the nucleotide sequences of SEQ ID Nos. 26 to 27, 30 to 31, 34 to 35, 38 to 39, 42 to 43, 46 to 47, 50 to 73, 76 to 90, 93 to 122, 125 to 154, 157 to 186, 189 to 218, 221 to 250, 253 to 282, 285 to 314, 317 to 346, and 354 to 356.

9. The oligonucleotide of claim 1 being selected from the group consisting of the nucleotide sequences of SEQ ID Nos. 26, 30, 31, 34, 38, 42, 46, 50, 51, 60, 64, 65, 68, 70, 78, 83, 85, 86, 87, 88, 89, 103, 109, 110, 114, 115, 116, 127, 132, 144, 164, 189, 205, 213, 216, 228, 260, 276, 292, 294, 297, 298, 312, 320, 324, 327, 328, 332, and 354.

10. The oligonucleotide of any one of the prior claims, comprising at least one nucleotide analog, e.g., without limitation, selected from the group consisting of 2′-O-methoxyethyl-modified oligonucleotides with phosphodiester or phosphorothioate backbone, and phosphorodamidate morpholino oligomers.

11. A composition comprising the oligonucleotide of any one of the prior claims and a pharmaceutically acceptable carrier.

12. A vector encoding the oligonucleotide of any one of the prior claims.

13. A method of generating or promoting exon skipping of an exon of interest during pre-mRNA splicing comprising contacting a pre-mRNA in a cell or subject with the oligonucleotide of any one of claims 1 to 10, the composition of claim 11, and/or the vector of claim 12 to restore the reading frame or generate a new splicing isoform.

14. The method of claim 13, wherein the ASO-mediated exon skipping manipulates expression of a gene of interest by inhibiting splicing of an exon, intron or a specific splice site of a gene of interest, leading to restoring the reading frame of a defective gene of interest, generating a different isoform of a gene of interest (e.g., a dominant negative isoform), skipping a toxic part of the gene, silencing the gene, and/or changing the structure and function of a gene.

15. A method of improving exon skipping efficacy and/or efficiency of a targeting sequence capable of hybridizing to a sequence selected from the group consisting of an exon of interest, a flanking intron sequence upstream of the exon of interest, a flanking intron sequence downstream of the exon of interest, an intron-exon junction upstream of the exon of interest, and an intron-exon junction downstream of the exon of interest in a cell or subject, comprising:
obtaining one or more oligonucleotides comprising the targeting sequence and a decoy sequence according to any one of claims 1 to 10.

16. The method of claim 15, further comprising screening the one or more oligonucleotides according to their exon skipping efficacies and/or efficiencies of the exon of interest.

17. The method of any one of claims 13 to 16, the gene of interest is selected from the group consisting of SMN1, SMN2, DMD, APP, CEP290, HER2, SCA3, PKM, and MDM4 genes.

18. The method of any one of claims 13 to 17, the exon of the gene of interest being selected from the group consisting of exon 7 of the endogenous SMN1 and SMN2 genes, exons 45, 51 and 52 of the endogenous DMD gene, exon 17 of the endogenous APP gene, exon 41 of the endogenous CEP290 gene, exon 19 of the endogenous HER2 gene, exon 10 of the SCA3 gene, exon 10 of the endogenous PKM gene, and exon 6 of the endogenous MDM4 gene.

19. The method of any one of claims 13 to 18, further comprising delivering to the cell or administering to the subject the oligonucleotide of any one of claims 1 to 10, the composition of claim 11, and/or the vector of claim 12.

20. A method of treating a disease and/or complications thereof in a subject comprising administering to the subject the oligonucleotide of any one of claims 1 to 10, the composition of claim 11, and/or the vector of claim 12, the oligonucleotide generating or promoting exon skipping of the exon of interest.

21. The method of claim 20, the oligonucleotide inhibiting splicing of an exon, intron or a specific splice site of a gene of interest, so as to restore the reading frame of a defective gene of interest, generate a different isoform of a gene of interest (e.g., a dominant negative isoform), skip a toxic part of the gene, silence the gene, and/or change the structure and function of a gene.

22. The method of claim 20 or claim 21, the disease and/or complications thereof being selected from the group consisting of diseases and/or complications thereof that may benefit from exon skipping on one or more genes selected from the group consisting of SMN1, SMN2, DMD, APP, CEP290, HER2, SCA3, PKM, and MDM4 genes.

23. The method of claim 22, the disease and/or complications thereof being selected from the group consisting of diseases and/or complications thereof that may benefit from exon skipping of one or more exons selected from the group consisting of exon 7 of the endogenous SMN1 and SMN2 genes, exons 45, 51 and 52 of the endogenous DMD gene, exon 17 of the endogenous APP gene, exon 41 of the endogenous CEP290 gene, exon 19 of the endogenous HER2 gene, exon 10 of the SCA3 gene, exon 10 of the endogenous PKM gene, and exon 6 of the endogenous MDM4 gene.

24. The method of claim 23, the disease and/or complications thereof being selected from the group consisting of Duchenne muscular dystrophy (DMD), Alzheimer's disease, Joubert Syndrome, spinocerebellar ataxia 3 (SCA3), breast cancer, HER2-positive biliary tract, colorectal, non-small-cell lung, bladder cancers, prostate cancer, lung cancer, cervix cancer, kidney cancer, papillary thyroid cancer, colon cancer, colorectal cancer, gliomas, ovarian cancer, gastric cancer, hepatoblastoma, fibrolamellar, hepatocellular, carcinoma, soft tissue sarcoma, osteosarcoma, chronic lymphocytic, leukemia, acute myeloid leukemia, mantle cell lymphoma, Pediatric Burkitt, lymphoma, salivary gland cancer, liver cancer, or melanoma.

25. The method of any one of claims 20 to 24, the oligonucleotide of any one of claims 1 to 10, the composition of claim 11, and/or the vector of claim 12 being administered at a therapeutically effective amount.

26. A kit comprising the oligonucleotide of any one of claims 1 to 10, the composition of claim 11, and/or the vector of claim 12, for use in the method of any one of claims 13 to 25.


SEQUENCE LISTING (357 sequences)

SEQ ID NO: 1-18 - skipped (18 entries)

SEQ ID NO: 19 - DNA, 10 bp, synthetic construct
  misc_feature 1..10: note = Synthetic
  source 1..10: mol_type = other DNA
caggtaagta

SEQ ID NO: 20 - DNA, 10 bp, synthetic construct
  misc_feature 1..10: note = Synthetic
  source 1..10: mol_type = other DNA
aggtaagtat

SEQ ID NO: 21 - DNA, 11 bp, synthetic construct
  misc_feature 1..11: note = Synthetic
  source 1..11: mol_type = other DNA
caggtaagtat

SEQ ID NO: 22 - DNA, 12 bp, synthetic construct
  misc_feature 1..12: note = Synthetic
  source 1..12: mol_type = other DNA
ccaggtaagtat

SEQ ID NO: 23 - DNA, 13 bp, synthetic construct
  misc_feature 1..13: note = Synthetic
  source 1..13: mol_type = other DNA
gccaggtaagtat

SEQ ID NO: 24 - RNA, 20 nt, synthetic construct
  misc_feature 1..20: note = Synthetic
  source 1..20: mol_type = other RNA
acttcctttattttccttac

SEQ ID NO: 25 - DNA, 20 bp, synthetic construct
  misc_feature 1..20: note = Synthetic
  source 1..20: mol_type = other DNA
gtaaggaaaataaaggaagt

SEQ ID NO: 26 - DNA, 31 bp, synthetic construct
  misc_feature 1..31: note = Synthetic
  source 1..31: mol_type = other DNA
caggtaagtatgtaaggaaaataaaggaagt

SEQ ID NO: 27 - DNA, 31 bp, synthetic construct
  misc_feature 1..31: note = Synthetic
  source 1..31: mol_type = other DNA
gtaaggaaaataaaggaagtcaggtaagtat

SEQ ID NO: 28 - RNA, 20 nt, synthetic construct
  misc_feature 1..20: note = Synthetic
  source 1..20: mol_type = other RNA
aaagaaggaaggtgctcaca

SEQ ID NO: 29 - DNA, 20 bp, synthetic construct
  misc_feature 1..20: note = Synthetic
  source 1..20: mol_type = other DNA
tgtgagcaccttccttcttt

SEQ ID NO: 30 - DNA, 31 bp, synthetic construct
  misc_feature 1..31: note = Synthetic
  source 1..31: mol_type = other DNA
caggtaagtattgtgagcaccttccttcttt

SEQ ID NO: 31 - DNA, 31 bp, synthetic construct
  misc_feature 1..31: note = Synthetic
  source 1..31: mol_type = other DNA
tgtgagcaccttccttctttcaggtaagtat

SEQ ID NO: 32 - RNA, 20 nt, synthetic construct
  misc_feature 1..20: note = Synthetic
  source 1..20: mol_type = other RNA
gtaagtctgccagcattatg

SEQ ID NO: 33 - DNA, 20 bp, synthetic construct
  misc_feature 1..20: note = Synthetic
  source 1..20: mol_type = other DNA
cataatgctggcagacttac

SEQ ID NO: 34 - DNA, 31 bp, synthetic construct
  misc_feature 1..31: note = Synthetic
  source 1..31: mol_type = other DNA
caggtaagtatcataatgctggcagacttac

SEQ ID NO: 35 - DNA, 31 bp, synthetic construct
  misc_feature 1..31: note = Synthetic
  source 1..31: mol_type = other DNA
cataatgctggcagacttaccaggtaagtat

SEQ ID NO: 36 - RNA, 19 nt, synthetic construct
  misc_feature 1..19: note = Synthetic
  source 1..19: mol_type = other RNA
aatgccatcttccttgatg

SEQ ID NO: 37 - DNA, 19 bp, synthetic construct
  misc_feature 1..19: note = Synthetic
  source 1..19: mol_type = other DNA
catcaaggaagatggcatt

SEQ ID NO: 38 - DNA, 30 bp, synthetic construct
  misc_feature 1..30: note = Synthetic
  source 1..30: mol_type = other DNA
caggtaagtatcatcaaggaagatggcatt

SEQ ID NO: 39 - DNA, 30 bp, synthetic construct
  misc_feature 1..30: note = Synthetic
  source 1..30: mol_type = other DNA
catcaaggaagatggcattcaggtaagtat

SEQ ID NO: 40 - RNA, 19 nt, synthetic construct
  misc_feature 1..19: note = Synthetic
  source 1..19: mol_type = other RNA
aactagaaatgccatcttc

SEQ ID NO: 41 - DNA, 19 bp, synthetic construct
  misc_feature 1..19: note = Synthetic
  source 1..19: mol_type = other DNA
gaagatggcatttctagtt

SEQ ID NO: 42 - DNA, 30 bp, synthetic construct
  misc_feature 1..30: note = Synthetic
  source 1..30: mol_type = other DNA
caggtaagtatgaagatggcatttctagtt

SEQ ID NO: 43 - DNA, 30 bp, synthetic construct
  misc_feature 1..30: note = Synthetic
  source 1..30: mol_type = other DNA
gaagatggcatttctagttcaggtaagtat

SEQ ID NO: 44 - RNA, 19 nt, synthetic construct
  misc_feature 1..19: note = Synthetic
  source 1..19: mol_type = other RNA
gccatctccaaactagaaa

SEQ ID NO: 45 - DNA, 19 bp, synthetic construct
  misc_feature 1..19: note = Synthetic
  source 1..19: mol_type = other DNA
tttctagtttggagatggc

SEQ ID NO: 46 - DNA, 30 bp, synthetic construct
  misc_feature 1..30: note = Synthetic
  source 1..30: mol_type = other DNA
caggtaagtattttctagtttggagatggc

SEQ ID NO: 47 - DNA, 30 bp, synthetic construct
  misc_feature 1..30: note = Synthetic
  source 1..30: mol_type = other DNA
tttctagtttggagatggccaggtaagtat

SEQ ID NO: 48 - RNA, 30 nt, synthetic construct
  misc_feature 1..30: note = Synthetic
  source 1..30: mol_type = other RNA
ctagaaatgccatcttccttgatgttggag

SEQ ID NO: 49 - DNA, 30 bp, synthetic construct
  misc_feature 1..30: note = Synthetic
  source 1..30: mol_type = other DNA
ctccaacatcaaggaagatggcatttctag

SEQ ID NO: 50 - DNA, 41 bp, synthetic construct
  misc_feature 1..41: note = Synthetic
  source 1..41: mol_type = other DNA
caggtaagtatctccaacatcaaggaagatggcatttctag

SEQ ID NO: 51 - DNA, 41 bp, synthetic construct
  misc_feature 1..41: note = Synthetic
  source 1..41: mol_type = other DNA
ctccaacatcaaggaagatggcatttctagcaggtaagtat

SEQ ID NO: 52 - DNA, 36 bp, synthetic construct
  misc_feature 1..36: note = Synthetic
  source 1..36: mol_type = other DNA
caggtactccaacatcaaggaagatggcatttctag

SEQ ID NO: 53 - DNA, 36 bp, synthetic construct
  misc_feature 1..36: note = Synthetic
  source 1..36: mol_type = other DNA
aggtaactccaacatcaaggaagatggcatttctag

SEQ ID NO: 54 - DNA, 36 bp, synthetic construct
  misc_feature 1..36: note = Synthetic
  source 1..36: mol_type = other DNA
ggtaagctccaacatcaaggaagatggcatttctag

SEQ ID NO: 55 - DNA, 36 bp, synthetic construct
  misc_feature 1..36: note = Synthetic
  source 1..36: mol_type = other DNA
gtaagtctccaacatcaaggaagatggcatttctag

SEQ ID NO: 56 - DNA, 36 bp, synthetic construct
  misc_feature 1..36: note = Synthetic
  source 1..36: mol_type = other DNA
taagtactccaacatcaaggaagatggcatttctag

SEQ ID NO: 57 - DNA, 36 bp, synthetic construct
  misc_feature 1..36: note = Synthetic
  source 1..36: mol_type = other DNA
aagtatctccaacatcaaggaagatggcatttctag

SEQ ID NO: 58 - DNA, 37 bp, synthetic construct
  misc_feature 1..37: note = Synthetic
  source 1..37: mol_type = other DNA
caggtaactccaacatcaaggaagatggcatttctag

SEQ ID NO: 59 - DNA, 37 bp, synthetic construct
  misc_feature 1..37: note = Synthetic
  source 1..37: mol_type = other DNA
aggtaagctccaacatcaaggaagatggcatttctag

SEQ ID NO: 60 - DNA, 37 bp, synthetic construct
  misc_feature 1..37: note = Synthetic
  source 1..37: mol_type = other DNA
ggtaagtctccaacatcaaggaagatggcatttctag

SEQ ID NO: 61 - DNA, 37 bp, synthetic construct
  misc_feature 1..37: note = Synthetic
  source 1..37: mol_type = other DNA
gtaagtactccaacatcaaggaagatggcatttctag

SEQ ID NO: 62 - DNA, 37 bp, synthetic construct
  misc_feature 1..37: note = Synthetic
  source 1..37: mol_type = other DNA
taagtatctccaacatcaaggaagatggcatttctag

SEQ ID NO: 63 - DNA, 38 bp, synthetic construct
  misc_feature 1..38: note = Synthetic
  source 1..38: mol_type = other DNA
caggtaagctccaacatcaaggaagatggcatttctag

SEQ ID NO: 64 - DNA, 38 bp, synthetic construct
  misc_feature 1..38: note = Synthetic
  source 1..38: mol_type = other DNA
aggtaagtctccaacatcaaggaagatggcatttctag

SEQ ID NO: 65 - DNA, 38 bp, synthetic construct
  misc_feature 1..38: note = Synthetic
  source 1..38: mol_type = other DNA
ggtaagtactccaacatcaaggaagatggcatttctag

SEQ ID NO: 66 - DNA, 38 bp, synthetic construct
  misc_feature 1..38: note = Synthetic
  source 1..38: mol_type = other DNA
gtaagtatctccaacatcaaggaagatggcatttctag

SEQ ID NO: 67 - DNA, 39 bp, synthetic construct
  misc_feature 1..39: note = Synthetic
  source 1..39: mol_type = other DNA
caggtaagtctccaacatcaaggaagatggcatttctag

SEQ ID NO: 68 - DNA, 39 bp, synthetic construct
  misc_feature 1..39: note = Synthetic
  source 1..39: mol_type = other DNA
aggtaagtactccaacatcaaggaagatggcatttctag

SEQ ID NO: 69 - DNA, 39 bp, synthetic construct
  misc_feature 1..39: note = Synthetic
  source 1..39: mol_type = other DNA
ggtaagtatctccaacatcaaggaagatggcatttctag

SEQ ID NO: 70 - DNA, 40 bp, synthetic construct
  misc_feature 1..40: note = Synthetic
  source 1..40: mol_type = other DNA
caggtaagtactccaacatcaaggaagatggcatttctag

SEQ ID NO: 71 - DNA, 40 bp, synthetic construct
  misc_feature 1..40: note = Synthetic
  source 1..40: mol_type = other DNA
aggtaagtatctccaacatcaaggaagatggcatttctag

SEQ ID NO: 72 - DNA, 42 bp, synthetic construct
  misc_feature 1..42: note = Synthetic
  source 1..42: mol_type = other DNA
ccaggtaagtatctccaacatcaaggaagatggcatttctag

SEQ ID NO: 73 - DNA, 43 bp, synthetic construct
  misc_feature 1..43: note = Synthetic
  source 1..43: mol_type = other DNA
gccaggtaagtatctccaacatcaaggaagatggcatttctag

SEQ ID NO: 74 - RNA, 21 nt, synthetic construct
  misc_feature 1..21: note = Synthetic
  source 1..21: mol_type = other RNA
aactgccatctccaaactaga

SEQ ID NO: 75 - DNA, 21 bp, synthetic construct
  misc_feature 1..21: note = Synthetic
  source 1..21: mol_type = other DNA
tctagtttggagatggcagtt

SEQ ID NO: 76 - DNA, 28 bp, synthetic construct
  misc_feature 1..28: note = Synthetic
  source 1..28: mol_type = other DNA
caggtaatctagtttggagatggcagtt

SEQ ID NO: 77 - DNA, 28 bp, synthetic construct
  misc_feature 1..28: note = Synthetic
  source 1..28: mol_type = other DNA
aggtaagtctagtttggagatggcagtt

SEQ ID NO: 78 - DNA, 28 bp, synthetic construct
  misc_feature 1..28: note = Synthetic
  source 1..28: mol_type = other DNA
ggtaagttctagtttggagatggcagtt

SEQ ID NO: 79 - DNA, 28 bp, synthetic construct
  misc_feature 1..28: note = Synthetic
  source 1..28: mol_type = other DNA
gtaagtatctagtttggagatggcagtt

SEQ ID NO: 80 - DNA, 28 bp, synthetic construct
  misc_feature 1..28: note = Synthetic
  source 1..28: mol_type = other DNA
taagtattctagtttggagatggcagtt

SEQ ID NO: 81 - DNA, 29 bp, synthetic construct
  misc_feature 1..29: note = Synthetic
  source 1..29: mol_type = other DNA
caggtaagtctagtttggagatggcagtt

SEQ ID NO: 82 - DNA, 29 bp, synthetic construct
  misc_feature 1..29: note = Synthetic
  source 1..29: mol_type = other DNA
aggtaagttctagtttggagatggcagtt

SEQ ID NO: 83 - DNA, 29 bp, synthetic construct
  misc_feature 1..29: note = Synthetic
  source 1..29: mol_type = other DNA
ggtaagtatctagtttggagatggcagtt

SEQ ID NO: 84 - DNA, 29 bp, synthetic construct
  misc_feature 1..29: note = Synthetic
  source 1..29: mol_type = other DNA
gtaagtattctagtttggagatggcagtt

SEQ ID NO: 85 - DNA, 30 bp, synthetic construct
  misc_feature 1..30: note = Synthetic
  source 1..30: mol_type = other DNA
caggtaagttctagtttggagatggcagtt

SEQ ID NO: 86 - DNA, 30 bp, synthetic construct
  misc_feature 1..30: note = Synthetic
  source 1..30: mol_type = other DNA
aggtaagtatctagtttggagatggcagtt

SEQ ID NO: 87 - DNA, 30 bp, synthetic construct
  misc_feature 1..30: note = Synthetic
  source 1..30: mol_type = other DNA
ggtaagtattctagtttggagatggcagtt

SEQ ID NO: 88 - DNA, 31 bp, synthetic construct
  misc_feature 1..31: note = Synthetic
  source 1..31: mol_type = other DNA
caggtaagtatctagtttggagatggcagtt

SEQ ID NO: 89 - DNA, 31 bp, synthetic construct
  misc_feature 1..31: note = Synthetic
  source 1..31: mol_type = other DNA
aggtaagtattctagtttggagatggcagtt

SEQ ID NO: 90 - DNA, 32 bp, synthetic construct
  misc_feature 1..32: note = Synthetic
  source 1..32: mol_type = other DNA
caggtaagtattctagtttggagatggcagtt

SEQ ID NO: 91 - RNA, 21 nt, synthetic construct
  misc_feature 1..21: note = Synthetic
  source 1..21: mol_type = other RNA
gaacaccttcagaaccggagg

SEQ ID NO: 92 - DNA, 21 bp, synthetic construct
  misc_feature 1..21: note = Synthetic
  source 1..21: mol_type = other DNA
cctccggttctgaaggtgttc

SEQ ID NO: 93 - DNA, 28 bp, synthetic construct
  misc_feature 1..28: note = Synthetic
  source 1..28: mol_type = other DNA
caggtaacctccggttctgaaggtgttc

SEQ ID NO: 94 - DNA, 28 bp, synthetic construct
  misc_feature 1..28: note = Synthetic
  source 1..28: mol_type = other DNA
aggtaagcctccggttctgaaggtgttc

SEQ ID NO: 95 - DNA, 28 bp, synthetic construct
  misc_feature 1..28: note = Synthetic
  source 1..28: mol_type = other DNA
ggtaagtcctccggttctgaaggtgttc

SEQ ID NO: 96 - DNA, 28 bp, synthetic construct
  misc_feature 1..28: note = Synthetic
  source 1..28: mol_type = other DNA
gtaagtacctccggttctgaaggtgttc

SEQ ID NO: 97 - DNA, 28 bp, synthetic construct
  misc_feature 1..28: note = Synthetic
  source 1..28: mol_type = other DNA
taagtatcctccggttctgaaggtgttc

SEQ ID NO: 98 - DNA, 29 bp, synthetic construct
  misc_feature 1..29: note = Synthetic
  source 1..29: mol_type = other DNA
caggtaagcctccggttctgaaggtgttc

SEQ ID NO: 99 - DNA, 29 bp, synthetic construct
  misc_feature 1..29: note = Synthetic
  source 1..29: mol_type = other DNA
aggtaagtcctccggttctgaaggtgttc

SEQ ID NO: 100 - DNA, 29 bp, synthetic construct
  misc_feature 1..29: note = Synthetic
  source 1..29: mol_type = other DNA
ggtaagtacctccggttctgaaggtgttc

SEQ ID NO: 101 - DNA, 29 bp, synthetic construct
  misc_feature 1..29: note = Synthetic
  source 1..29: mol_type = other DNA
gtaagtatcctccggttctgaaggtgttc

SEQ ID NO: 102 - DNA, 30 bp, synthetic construct
  misc_feature 1..30: note = Synthetic
  source 1..30: mol_type = other DNA
caggtaagtcctccggttctgaaggtgttc

SEQ ID NO: 103 - DNA, 30 bp, synthetic construct
  misc_feature 1..30: note = Synthetic
  source 1..30: mol_type = other DNA
aggtaagtacctccggttctgaaggtgttc

SEQ ID NO: 104 - DNA, 30 bp, synthetic construct
  misc_feature 1..30: note = Synthetic
  source 1..30: mol_type = other DNA
ggtaagtatcctccggttctgaaggtgttc

SEQ ID NO: 105 - DNA, 31 bp, synthetic construct
  misc_feature 1..31: note = Synthetic
  source 1..31: mol_type = other DNA
caggtaagtacctccggttctgaaggtgttc

SEQ ID NO: 106 - DNA, 31 bp, synthetic construct
  misc_feature 1..31: note = Synthetic
  source 1..31: mol_type = other DNA
aggtaagtatcctccggttctgaaggtgttc

SEQ ID NO: 107 - DNA, 32 bp, synthetic construct
  misc_feature 1..32: note = Synthetic
  source 1..32: mol_type = other DNA
caggtaagtatcctccggttctgaaggtgttc

SEQ ID NO: 108 - DNA, 28 bp, synthetic construct
  misc_feature 1..28: note = Synthetic
  source 1..28: mol_type = other DNA
cctccggttctgaaggtgttccaggtaa

SEQ ID NO: 109 - DNA, 28 bp, synthetic construct
  misc_feature 1..28: note = Synthetic
  source 1..28: mol_type = other DNA
cctccggttctgaaggtgttcaggtaag

SEQ ID NO: 110 - DNA, 28 bp, synthetic construct
  misc_feature 1..28: note = Synthetic
  source 1..28: mol_type = other DNA
cctccggttctgaaggtgttcggtaagt

SEQ ID NO: 111 - DNA, 28 bp, synthetic construct
  misc_feature 1..28: note = Synthetic
  source 1..28: mol_type = other DNA
cctccggttctgaaggtgttcgtaagta

SEQ ID NO: 112 - DNA, 28 bp, synthetic construct
  misc_feature 1..28: note = Synthetic
  source 1..28: mol_type = other DNA
cctccggttctgaaggtgttctaagtat

SEQ ID NO: 113 - DNA, 29 bp, synthetic construct
  misc_feature 1..29: note = Synthetic
  source 1..29: mol_type = other DNA
cctccggttctgaaggtgttccaggtaag

SEQ ID NO: 114 - DNA, 29 bp, synthetic construct
  misc_feature 1..29: note = Synthetic
  source 1..29: mol_type = other DNA
cctccggttctgaaggtgttcaggtaagt

SEQ ID NO: 115 - DNA, 29 bp, synthetic construct
  misc_feature 1..29: note = Synthetic
  source 1..29: mol_type = other DNA
cctccggttctgaaggtgttcggtaagta

SEQ ID NO: 116 - DNA, 29 bp, synthetic construct
  misc_feature 1..29: note = Synthetic
  source 1..29: mol_type = other DNA
cctccggttctgaaggtgttcgtaagtat

SEQ ID NO: 117 - DNA, 30 bp, synthetic construct
  misc_feature 1..30: note = Synthetic
  source 1..30: mol_type = other DNA
cctccggttctgaaggtgttccaggtaagt

SEQ ID NO: 118 - DNA, 30 bp, synthetic construct
  misc_feature 1..30: note = Synthetic
  source 1..30: mol_type = other DNA
cctccggttctgaaggtgttcaggtaagta

SEQ ID NO: 119 - DNA, 30 bp, synthetic construct
  misc_feature 1..30: note = Synthetic
  source 1..30: mol_type = other DNA
cctccggttctgaaggtgttcggtaagtat

SEQ ID NO: 120 - DNA, 31 bp, synthetic construct
  misc_feature 1..31: note = Synthetic
  source 1..31: mol_type = other DNA
cctccggttctgaaggtgttccaggtaagta

SEQ ID NO: 121 - DNA, 31 bp, synthetic construct
  misc_feature 1..31: note = Synthetic
  source 1..31: mol_type = other DNA
cctccggttctgaaggtgttcaggtaagtat

SEQ ID NO: 122 - DNA, 32 bp, synthetic construct
  misc_feature 1..32: note = Synthetic
  source 1..32: mol_type = other DNA
cctccggttctgaaggtgttccaggtaagtat

SEQ ID NO: 123 - RNA, 22 nt, synthetic construct
  misc_feature 1..22: note = Synthetic
  source 1..22: mol_type = other RNA
cagaaaaaagaggtagggcgac

SEQ ID NO: 124 - DNA, 22 bp, synthetic construct
  misc_feature 1..22: note = Synthetic
  source 1..22: mol_type = other DNA
gtcgccctacctcttttttctg

SEQ ID NO: 125 - DNA, 29 bp, synthetic construct
  misc_feature 1..29: note = Synthetic
  source 1..29: mol_type = other DNA
caggtaagtcgccctacctcttttttctg

SEQ ID NO: 126 - DNA, 29 bp, synthetic construct
  misc_feature 1..29: note = Synthetic
  source 1..29: mol_type = other DNA
aggtaaggtcgccctacctcttttttctg

SEQ ID NO: 127 - DNA, 29 bp, synthetic construct
  misc_feature 1..29: note = Synthetic
  source 1..29: mol_type = other DNA
ggtaagtgtcgccctacctcttttttctg

SEQ ID NO: 128 - DNA, 29 bp, synthetic construct
  misc_feature 1..29: note = Synthetic
  source 1..29: mol_type = other DNA
gtaagtagtcgccctacctcttttttctg

SEQ ID NO: 129 - DNA, 29 bp, synthetic construct
  misc_feature 1..29: note = Synthetic
  source 1..29: mol_type = other DNA
taagtatgtcgccctacctcttttttctg

SEQ ID NO: 130 - DNA, 30 bp, synthetic construct
  misc_feature 1..30: note = Synthetic
  source 1..30: mol_type = other DNA
caggtaaggtcgccctacctcttttttctg

SEQ ID NO: 131 - DNA, 30 bp, synthetic construct
  misc_feature 1..30: note = Synthetic
  source 1..30: mol_type = other DNA
aggtaagtgtcgccctacctcttttttctg

SEQ ID NO: 132 - DNA, 30 bp, synthetic construct
  misc_feature 1..30: note = Synthetic
  source 1..30: mol_type = other DNA
ggtaagtagtcgccctacctcttttttctg

SEQ ID NO: 133 - DNA, 30 bp, synthetic construct
  misc_feature 1..30: note = Synthetic
  source 1..30: mol_type = other DNA
gtaagtatgtcgccctacctcttttttctg

SEQ ID NO: 134 - DNA, 31 bp, synthetic construct
  misc_feature 1..31: note = Synthetic
  source 1..31: mol_type = other DNA
caggtaagtgtcgccctacctcttttttctg

SEQ ID NO: 135 - DNA, 31 bp, synthetic construct
  misc_feature 1..31: note = Synthetic
  source 1..31: mol_type = other DNA
aggtaagtagtcgccctacctcttttttctg

SEQ ID NO: 136 - DNA, 31 bp, synthetic construct
  misc_feature 1..31: note = Synthetic
  source 1..31: mol_type = other DNA
ggtaagtatgtcgccctacctcttttttctg

SEQ ID NO: 137 - DNA, 32 bp, synthetic construct
  misc_feature 1..32: note = Synthetic
  source 1..32: mol_type = other DNA
caggtaagtagtcgccctacctcttttttctg

SEQ ID NO: 138 - DNA, 32 bp, synthetic construct
  misc_feature 1..32: note = Synthetic
  source 1..32: mol_type = other DNA
aggtaagtatgtcgccctacctcttttttctg

SEQ ID NO: 139 - DNA, 33 bp, synthetic construct
  misc_feature 1..33: note = Synthetic
  source 1..33: mol_type = other DNA
caggtaagtatgtcgccctacctcttttttctg

SEQ ID NO: 140 - DNA, 29 bp, synthetic construct
  misc_feature 1..29: note = Synthetic
  source 1..29: mol_type = other DNA
gtcgccctacctcttttttctgcaggtaa

SEQ ID NO: 141 - DNA, 29 bp, synthetic construct
  misc_feature 1..29: note = Synthetic
  source 1..29: mol_type = other DNA
gtcgccctacctcttttttctgaggtaag

SEQ ID NO: 142 - DNA, 29 bp, synthetic construct
  misc_feature 1..29: note = Synthetic
  source 1..29: mol_type = other DNA
gtcgccctacctcttttttctgggtaagt

SEQ ID NO: 143 - DNA, 29 bp, synthetic construct
  misc_feature 1..29: note = Synthetic
  source 1..29: mol_type = other DNA
gtcgccctacctcttttttctggtaagta

SEQ ID NO: 144 - DNA, 29 bp, synthetic construct
  misc_feature 1..29: note = Synthetic
  source 1..29: mol_type = other DNA
gtcgccctacctcttttttctgtaagtat

SEQ ID NO: 145 - DNA, 30 bp, synthetic construct
  misc_feature 1..30: note = Synthetic
  source 1..30: mol_type = other DNA
gtcgccctacctcttttttctgcaggtaag

SEQ ID NO: 146 - DNA, 30 bp, synthetic construct
  misc_feature 1..30: note = Synthetic
  source 1..30: mol_type = other DNA
gtcgccctacctcttttttctgaggtaagt

SEQ ID NO: 147 - DNA, 30 bp, synthetic construct
  misc_feature 1..30: note = Synthetic
  source 1..30: mol_type = other DNA
gtcgccctacctcttttttctgggtaagta

SEQ ID NO: 148 - DNA, 30 bp, synthetic construct
  misc_feature 1..30: note = Synthetic
  source 1..30: mol_type = other DNA
gtcgccctacctcttttttctggtaagtat

SEQ ID NO: 149 - DNA, 31 bp, synthetic construct
  misc_feature 1..31: note = Synthetic
  source 1..31: mol_type = other DNA
gtcgccctacctcttttttctgcaggtaagt

SEQ ID NO: 150 - DNA, 31 bp, synthetic construct
  misc_feature 1..31: note = Synthetic
  source 1..31: mol_type = other DNA
gtcgccctacctcttttttctgaggtaagta

SEQ ID NO: 151 - DNA, 31 bp, synthetic construct
  misc_feature 1..31: note = Synthetic
  source 1..31: mol_type = other DNA
gtcgccctacctcttttttctgggtaagtat

SEQ ID NO: 152 - DNA, 32 bp, synthetic construct
  misc_feature 1..32: note = Synthetic
  source 1..32: mol_type = other DNA
gtcgccctacctcttttttctgcaggtaagta

SEQ ID NO: 153 - DNA, 32 bp, synthetic construct
  misc_feature 1..32: note = Synthetic
  source 1..32: mol_type = other DNA
gtcgccctacctcttttttctgaggtaagtat

SEQ ID NO: 154 - DNA, 33 bp, synthetic construct
  misc_feature 1..33: note = Synthetic
  source 1..33: mol_type = other DNA
gtcgccctacctcttttttctgcaggtaagtat

SEQ ID NO: 155 - RNA, 18 nt, synthetic construct
  misc_feature 1..18: note = Synthetic
  source 1..18: mol_type = other RNA
tcaaggtgttctttgcag

SEQ ID NO: 156 - DNA, 18 bp, synthetic construct
  misc_feature 1..18: note = Synthetic
  source 1..18: mol_type = other DNA
ctgcaaagaacaccttga

SEQ ID NO: 157 - DNA, 25 bp, synthetic construct
  misc_feature 1..25: note = Synthetic
  source 1..25: mol_type = other DNA
caggtaactgcaaagaacaccttga

SEQ ID NO: 158 - DNA, 25 bp, synthetic construct
  misc_feature 1..25: note = Synthetic
  source 1..25: mol_type = other DNA
aggtaagctgcaaagaacaccttga

SEQ ID NO: 159 - DNA, 25 bp, synthetic construct
  misc_feature 1..25: note = Synthetic
  source 1..25: mol_type = other DNA
ggtaagtctgcaaagaacaccttga

SEQ ID NO: 160 - DNA, 25 bp, synthetic construct
  misc_feature 1..25: note = Synthetic
  source 1..25: mol_type = other DNA
gtaagtactgcaaagaacaccttga

SEQ ID NO: 161 - DNA, 25 bp, synthetic construct
  misc_feature 1..25: note = Synthetic
  source 1..25: mol_type = other DNA
taagtatctgcaaagaacaccttga

SEQ ID NO: 162 - DNA, 26 bp, synthetic construct
  misc_feature 1..26: note = Synthetic
  source 1..26: mol_type = other DNA
caggtaagctgcaaagaacaccttga

SEQ ID NO: 163 - DNA, 26 bp, synthetic construct
  misc_feature 1..26: note = Synthetic
  source 1..26: mol_type = other DNA
aggtaagtctgcaaagaacaccttga

SEQ ID NO: 164 - DNA, 26 bp, synthetic construct
  misc_feature 1..26: note = Synthetic
  source 1..26: mol_type = other DNA
ggtaagtactgcaaagaacaccttga

SEQ ID NO: 165 - DNA, 26 bp, synthetic construct
  misc_feature 1..26: note = Synthetic
  source 1..26: mol_type = other DNA
gtaagtatctgcaaagaacaccttga

SEQ ID NO: 166 - DNA, 27 bp, synthetic construct
  misc_feature 1..27: note = Synthetic
  source 1..27: mol_type = other DNA
caggtaagtctgcaaagaacaccttga

SEQ ID NO: 167 - DNA, 27 bp, synthetic construct
  misc_feature 1..27: note = Synthetic
  source 1..27: mol_type = other DNA
aggtaagtactgcaaagaacaccttga

SEQ ID NO: 168 - DNA, 27 bp, synthetic construct
  misc_feature 1..27: note = Synthetic
  source 1..27: mol_type = other DNA
ggtaagtatctgcaaagaacaccttga

SEQ ID NO: 169 - DNA, 28 bp, synthetic construct
  misc_feature 1..28: note = Synthetic
  source 1..28: mol_type = other DNA
caggtaagtactgcaaagaacaccttga

SEQ ID NO: 170 - DNA, 28 bp, synthetic construct
  misc_feature 1..28: note = Synthetic
  source 1..28: mol_type = other DNA
aggtaagtatctgcaaagaacaccttga

SEQ ID NO: 171 - DNA, 29 bp, synthetic construct
  misc_feature 1..29: note = Synthetic
  source 1..29: mol_type = other DNA
caggtaagtatctgcaaagaacaccttga

SEQ ID NO: 172 - DNA, 25 bp, synthetic construct
  misc_feature 1..25: note = Synthetic
  source 1..25: mol_type = other DNA
ctgcaaagaacaccttgacaggtaa

SEQ ID NO: 173 - DNA, 25 bp, synthetic construct
  misc_feature 1..25: note = Synthetic
  source 1..25: mol_type = other DNA
ctgcaaagaacaccttgaaggtaag

SEQ ID NO: 174 - DNA, 25 bp, synthetic construct
  misc_feature 1..25: note = Synthetic
  source 1..25: mol_type = other DNA
ctgcaaagaacaccttgaggtaagt

SEQ ID NO: 175 - DNA, 25 bp, synthetic construct
  misc_feature 1..25: note = Synthetic
  source 1..25: mol_type = other DNA
ctgcaaagaacaccttgagtaagta

SEQ ID NO: 176 - DNA, 25 bp, synthetic construct
  misc_feature 1..25: note = Synthetic
  source 1..25: mol_type = other DNA
ctgcaaagaacaccttgataagtat

SEQ ID NO: 177 - DNA, 26 bp, synthetic construct
  misc_feature 1..26: note = Synthetic
  source 1..26: mol_type = other DNA
ctgcaaagaacaccttgacaggtaag

SEQ ID NO: 178 - DNA, 26 bp, synthetic construct
  misc_feature 1..26: note = Synthetic
  source 1..26: mol_type = other DNA
ctgcaaagaacaccttgaaggtaagt

SEQ ID NO: 179 - DNA, 26 bp, synthetic construct
  misc_feature 1..26: note = Synthetic
  source 1..26: mol_type = other DNA
ctgcaaagaacaccttgaggtaagta

SEQ ID NO: 180 - DNA, 26 bp, synthetic construct
  misc_feature 1..26: note = Synthetic
  source 1..26: mol_type = other DNA
ctgcaaagaacaccttgagtaagtat

SEQ ID NO: 181 - DNA, 27 bp, synthetic construct
  misc_feature 1..27: note = Synthetic
  source 1..27: mol_type = other DNA
ctgcaaagaacaccttgacaggtaagt

SEQ ID NO: 182 - DNA, 27 bp, synthetic construct
  misc_feature 1..27: note = Synthetic
  source 1..27: mol_type = other DNA
ctgcaaagaacaccttgaaggtaagta

SEQ ID NO: 183 - DNA, 27 bp, synthetic construct
  misc_feature 1..27: note = Synthetic
  source 1..27: mol_type = other DNA
ctgcaaagaacaccttgaggtaagtat

SEQ ID NO: 184 - DNA, 28 bp, synthetic construct
  misc_feature 1..28: note = Synthetic
  source 1..28: mol_type = other DNA
ctgcaaagaacaccttgacaggtaagta

SEQ ID NO: 185 - DNA, 28 bp, synthetic construct
  misc_feature 1..28: note = Synthetic
  source 1..28: mol_type = other DNA
ctgcaaagaacaccttgaaggtaagtat

SEQ ID NO: 186 - DNA, 29 bp, synthetic construct
  misc_feature 1..29: note = Synthetic
  source 1..29: mol_type = other DNA
ctgcaaagaacaccttgacaggtaagtat

SEQ ID NO: 187 - RNA, 20 nt, synthetic construct
  misc_feature 1..20: note = Synthetic
  source 1..20: mol_type = other RNA
aaagtctaattgaagaactc

SEQ ID NO: 188 - DNA, 20 bp, synthetic construct
  misc_feature 1..20: note = Synthetic
  source 1..20: mol_type = other DNA
gagttcttcaattagacttt

SEQ ID NO: 189 - DNA, 27 bp, synthetic construct
  misc_feature 1..27: note = Synthetic
  source 1..27: mol_type = other DNA
caggtaagagttcttcaattagacttt

SEQ ID NO: 190 - DNA, 27 bp, synthetic construct
  misc_feature 1..27: note = Synthetic
  source 1..27: mol_type = other DNA
aggtaaggagttcttcaattagacttt

SEQ ID NO: 191 - DNA, 27 bp, synthetic construct
  misc_feature 1..27: note = Synthetic
  source 1..27: mol_type = other DNA
ggtaagtgagttcttcaattagacttt

SEQ ID NO: 192 - DNA, 27 bp, synthetic construct
  misc_feature 1..27: note = Synthetic
  source 1..27: mol_type = other DNA
gtaagtagagttcttcaattagacttt

SEQ ID NO: 193 - DNA, 27 bp, synthetic construct
  misc_feature 1..27: note = Synthetic
  source 1..27: mol_type = other DNA
taagtatgagttcttcaattagacttt

SEQ ID NO: 194 - DNA, 28 bp, synthetic construct
  misc_feature 1..28: note = Synthetic
  source 1..28: mol_type = other DNA
caggtaaggagttcttcaattagacttt

SEQ ID NO: 195 - DNA, 28 bp, synthetic construct
  misc_feature 1..28: note = Synthetic
  source 1..28: mol_type = other DNA
aggtaagtgagttcttcaattagacttt

SEQ ID NO: 196 - DNA, 28 bp, synthetic construct
  misc_feature 1..28: note = Synthetic
  source 1..28: mol_type = other DNA
ggtaagtagagttcttcaattagacttt

SEQ ID NO: 197 - DNA, 28 bp, synthetic construct
  misc_feature 1..28: note = Synthetic
  source 1..28: mol_type = other DNA
gtaagtatgagttcttcaattagacttt

SEQ ID NO: 198 - DNA, 29 bp, synthetic construct
  misc_feature 1..29: note = Synthetic
  source 1..29: mol_type = other DNA
caggtaagtgagttcttcaattagacttt

SEQ ID NO: 199 - DNA, 29 bp, synthetic construct
  misc_feature 1..29: note = Synthetic
  source 1..29: mol_type = other DNA
aggtaagtagagttcttcaattagacttt

SEQ ID NO: 200 - DNA, 29 bp, synthetic construct
  misc_feature 1..29: note = Synthetic
  source 1..29: mol_type = other DNA
ggtaagtatgagttcttcaattagacttt

SEQ ID NO: 201 - DNA, 30 bp, synthetic construct
  misc_feature 1..30: note = Synthetic
  source 1..30: mol_type = other DNA
caggtaagtagagttcttcaattagacttt

SEQ ID NO: 202 - DNA, 30 bp, synthetic construct
  misc_feature 1..30: note = Synthetic
  source 1..30: mol_type = other DNA
aggtaagtatgagttcttcaattagacttt

SEQ ID NO: 203 - DNA, 31 bp, synthetic construct
  misc_feature 1..31: note = Synthetic
  source 1..31: mol_type = other DNA
caggtaagtatgagttcttcaattagacttt

SEQ ID NO: 204 - DNA, 27 bp, synthetic construct
  misc_feature 1..27: note = Synthetic
  source 1..27: mol_type = other DNA
gagttcttcaattagactttcaggtaa

SEQ ID NO: 205 - DNA, 27 bp, synthetic construct
  misc_feature 1..27: note = Synthetic
  source 1..27: mol_type = other DNA
gagttcttcaattagactttaggtaag

SEQ ID NO: 206 - DNA, 27 bp, synthetic construct
  misc_feature 1..27: note = Synthetic
  source 1..27: mol_type = other DNA
gagttcttcaattagactttggtaagt

SEQ ID NO: 207 - DNA, 27 bp, synthetic construct
  misc_feature 1..27: note = Synthetic
  source 1..27: mol_type = other DNA
gagttcttcaattagactttgtaagta

SEQ ID NO: 208 - DNA, 27 bp, synthetic construct
  misc_feature 1..27: note = Synthetic
  source 1..27: mol_type = other DNA
gagttcttcaattagacttttaagtat

SEQ ID NO: 209 - DNA, 28 bp, synthetic construct
  misc_feature 1..28: note = Synthetic
  source 1..28: mol_type = other DNA
gagttcttcaattagactttcaggtaag

SEQ ID NO: 210 - DNA, 28 bp, synthetic construct
  misc_feature 1..28: note = Synthetic
  source 1..28: mol_type = other DNA
gagttcttcaattagactttaggtaagt

SEQ ID NO: 211 - DNA, 28 bp, synthetic construct
  misc_feature 1..28: note = Synthetic
  source 1..28: mol_type = other DNA
gagttcttcaattagactttggtaagta

SEQ ID NO: 212 - DNA, 28 bp, synthetic construct
  misc_feature 1..28: note = Synthetic
  source 1..28: mol_type = other DNA
gagttcttcaattagactttgtaagtat

SEQ ID NO: 213 - DNA, 29 bp, synthetic construct
  misc_feature 1..29: note = Synthetic
  source 1..29: mol_type = other DNA
gagttcttcaattagactttcaggtaagt

SEQ ID NO: 214 - DNA, 29 bp, synthetic construct
  misc_feature 1..29: note = Synthetic
  source 1..29: mol_type = other DNA
gagttcttcaattagactttaggtaagta

SEQ ID NO: 215 - DNA, 29 bp, synthetic construct
  misc_feature 1..29: note = Synthetic
  source 1..29: mol_type = other DNA
gagttcttcaattagactttggtaagtat

SEQ ID NO: 216 - DNA, 30 bp, synthetic construct
  misc_feature 1..30: note = Synthetic
  source 1..30: mol_type = other DNA
gagttcttcaattagactttcaggtaagta

SEQ ID NO: 217 - DNA, 30 bp, synthetic construct
  misc_feature 1..30: note = Synthetic
  source 1..30: mol_type = other DNA
gagttcttcaattagactttaggtaagtat

SEQ ID NO: 218 - DNA, 31 bp, synthetic construct
  misc_feature 1..31: note = Synthetic
  source 1..31: mol_type = other DNA
gagttcttcaattagactttcaggtaagtat

SEQ ID NO: 219 - RNA, 15 nt, synthetic construct
  misc_feature 1..15: note = Synthetic
  source 1..15: mol_type = other RNA
ccagggcatctggat

SEQ ID NO: 220 - DNA, 15 bp, synthetic construct
  misc_feature 1..15: note = Synthetic
  source 1..15: mol_type = other DNA
atccagatgccctgg

SEQ ID NO: 221 - DNA, 22 bp, synthetic construct
  misc_feature 1..22: note = Synthetic
  source 1..22: mol_type = other DNA
caggtaaatccagatgccctgg

SEQ ID NO: 222 - DNA, 22 bp, synthetic construct
  misc_feature 1..22: note = Synthetic
  source 1..22: mol_type = other DNA
aggtaagatccagatgccctgg

SEQ ID NO: 223 - DNA, 22 bp, synthetic construct
  misc_feature 1..22: note = Synthetic
  source 1..22: mol_type = other DNA
ggtaagtatccagatgccctgg

SEQ ID NO: 224 - DNA, 22 bp, synthetic construct
  misc_feature 1..22: note = Synthetic
  source 1..22: mol_type = other DNA
gtaagtaatccagatgccctgg

SEQ ID NO: 225 - DNA, 22 bp, synthetic construct
  misc_feature 1..22: note = Synthetic
  source 1..22: mol_type = other DNA
taagtatatccagatgccctgg

SEQ ID NO: 226 - DNA, 23 bp, synthetic construct
  misc_feature 1..23: note = Synthetic
  source 1..23: mol_type = other DNA
caggtaagatccagatgccctgg

SEQ ID NO: 227 - DNA, 23 bp, synthetic construct
  misc_feature 1..23: note = Synthetic
  source 1..23: mol_type = other DNA
aggtaagtatccagatgccctgg

SEQ ID NO: 228 - DNA, 23 bp, synthetic construct
  misc_feature 1..23: note = Synthetic
  source 1..23: mol_type = other DNA
ggtaagtaatccagatgccctgg

SEQ ID NO: 229 - DNA, 23 bp, synthetic construct
  misc_feature 1..23: note = Synthetic
  source 1..23: mol_type = other DNA
gtaagtatatccagatgccctgg

SEQ ID NO: 230 - DNA, 24 bp, synthetic construct
  misc_feature 1..24: note = Synthetic
  source 1..24: mol_type = other DNA
caggtaagtatccagatgccctgg

SEQ ID NO: 231 - DNA, 24 bp, synthetic construct
  misc_feature 1..24: note = Synthetic
  source 1..24: mol_type = other DNA
aggtaagtaatccagatgccctgg

SEQ ID NO: 232 - DNA, 24 bp, synthetic construct
  misc_feature 1..24: note = Synthetic
  source 1..24: mol_type = other DNA
ggtaagtatatccagatgccctgg

SEQ ID NO: 233 - DNA, 25 bp, synthetic construct
  misc_feature 1..25: note = Synthetic
  source 1..25: mol_type = other DNA
caggtaagtaatccagatgccctgg

SEQ ID NO: 234 - DNA, 25 bp, synthetic construct
  misc_feature 1..25: note = Synthetic
  source 1..25: mol_type = other DNA
aggtaagtatatccagatgccctgg

SEQ ID NO: 235 - DNA, 26 bp, synthetic construct
  misc_feature 1..26: note = Synthetic
  source 1..26: mol_type = other DNA
caggtaagtatatccagatgccctgg

SEQ ID NO: 236 - DNA, 22 bp, synthetic construct
  misc_feature 1..22: note = Synthetic
  source 1..22: mol_type = other DNA
atccagatgccctggcaggtaa

SEQ ID NO: 237 - DNA, 22 bp, synthetic construct
  misc_feature 1..22: note = Synthetic
  source 1..22: mol_type = other DNA
atccagatgccctggaggtaag

SEQ ID NO: 238 - DNA, 22 bp, synthetic construct
  misc_feature 1..22: note = Synthetic
  source 1..22: mol_type = other DNA
atccagatgccctggggtaagt

SEQ ID NO: 239 - DNA, 22 bp, synthetic construct
  misc_feature 1..22: note = Synthetic
  source 1..22: mol_type = other DNA
atccagatgccctgggtaagta

SEQ ID NO: 240 - DNA, 22 bp, synthetic construct
  misc_feature 1..22: note = Synthetic
  source 1..22: mol_type = other DNA
atccagatgccctggtaagtat

SEQ ID NO: 241 - DNA, 23 bp, synthetic construct
  misc_feature 1..23: note = Synthetic
  source 1..23: mol_type = other DNA
atccagatgccctggcaggtaag

SEQ ID NO: 242 - DNA, 23 bp, synthetic construct
  misc_feature 1..23: note = Synthetic
  source 1..23: mol_type = other DNA
atccagatgccctggaggtaagt

SEQ ID NO: 243 - DNA, 23 bp, synthetic construct
  misc_feature 1..23: note = Synthetic
  source 1..23: mol_type = other DNA
atccagatgccctggggtaagta

SEQ ID NO: 244 - DNA, 23 bp, synthetic construct
  misc_feature 1..23: note = Synthetic
  source 1..23: mol_type = other DNA
atccagatgccctgggtaagtat

SEQ ID NO: 245 - DNA, 24 bp, synthetic construct
  misc_feature 1..24: note = Synthetic
  source 1..24: mol_type = other DNA
atccagatgccctggcaggtaagt

SEQ ID NO: 246 - DNA, 24 bp, synthetic construct
  misc_feature 1..24: note = Synthetic
  source 1..24: mol_type = other DNA
atccagatgccctggaggtaagta

SEQ ID NO: 247 - DNA, 24 bp, synthetic construct
  misc_feature 1..24: note = Synthetic
  source 1..24: mol_type = other DNA
atccagatgccctggggtaagtat

SEQ ID NO: 248 - DNA, 25 bp, synthetic construct
  misc_feature 1..25: note = Synthetic
  source 1..25: mol_type = other DNA
atccagatgccctggcaggtaagta

SEQ ID NO: 249 - DNA, 25 bp, synthetic construct
  misc_feature 1..25: note = Synthetic
  source 1..25: mol_type = other DNA
atccagatgccctggaggtaagtat

SEQ ID NO: 250 - DNA, 26 bp, synthetic construct
  misc_feature 1..26: note = Synthetic
  source 1..26: mol_type = other DNA
atccagatgccctggcaggtaagtat

SEQ ID NO: 251 - DNA, 21 bp, synthetic construct
  misc_feature 1..21: note = Synthetic
  source 1..21: mol_type = other DNA
cagcagcagcagcagcagcag

SEQ ID NO: 252 - DNA, 21 bp, synthetic construct
  misc_feature 1..21: note = Synthetic
  source 1..21: mol_type = other DNA
ctgctgctgctgctgctgctg

SEQ ID NO: 253 - DNA, 28 bp, synthetic construct
  misc_feature 1..28: note = Synthetic
  source 1..28: mol_type = other DNA
caggtaactgctgctgctgctgctgctg

SEQ ID NO: 254 - DNA, 28 bp, synthetic construct
  misc_feature 1..28: note = Synthetic
  source 1..28: mol_type = other DNA
aggtaagctgctgctgctgctgctgctg

SEQ ID NO: 255 - DNA, 28 bp, synthetic construct
  misc_feature 1..28: note = Synthetic
  source 1..28: mol_type = other DNA
ggtaagtctgctgctgctgctgctgctg

SEQ ID NO: 256 - DNA, 28 bp, synthetic construct
  misc_feature 1..28: note = Synthetic
  source 1..28: mol_type = other DNA
gtaagtactgctgctgctgctgctgctg

SEQ ID NO: 257 - DNA, 28 bp, synthetic construct
  misc_feature 1..28: note = Synthetic
  source 1..28: mol_type = other DNA
taagtatctgctgctgctgctgctgctg

SEQ ID NO: 258 - DNA, 29 bp, synthetic construct
  misc_feature 1..29: note = Synthetic
  source 1..29: mol_type = other DNA
caggtaagctgctgctgctgctgctgctg

SEQ ID NO: 259 - DNA, 29 bp, synthetic construct
  misc_feature 1..29: note = Synthetic
  source 1..29: mol_type = other DNA
aggtaagtctgctgctgctgctgctgctg

SEQ ID NO: 260 - DNA, 29 bp, synthetic construct
  misc_feature 1..29: note = Synthetic
  source 1..29: mol_type = other DNA
ggtaagtactgctgctgctgctgctgctg

SEQ ID NO: 261 - DNA, 29 bp, synthetic construct
  misc_feature 1..29: note = Synthetic
  source 1..29: mol_type = other DNA
gtaagtatctgctgctgctgctgctgctg

SEQ ID NO: 262 - DNA, 30 bp, synthetic construct
  misc_feature 1..30: note = Synthetic
  source 1..30: mol_type = other DNA
caggtaagtctgctgctgctgctgctgctg

SEQ ID NO: 263 - DNA, 30 bp, synthetic construct
  misc_feature 1..30: note = Synthetic
  source 1..30: mol_type = other DNA
aggtaagtactgctgctgctgctgctgctg

SEQ ID NO: 264 - DNA, 30 bp, synthetic construct
  misc_feature 1..30: note = Synthetic
  source 1..30: mol_type = other DNA
ggtaagtatctgctgctgctgctgctgctg

SEQ ID NO: 265 - DNA, 31 bp, synthetic construct
  misc_feature 1..31: note = Synthetic
  source 1..31: mol_type = other DNA
caggtaagtactgctgctgctgctgctgctg

SEQ ID NO: 266 - DNA, 31 bp, synthetic construct
  misc_feature 1..31: note = Synthetic
  source 1..31: mol_type = other DNA
aggtaagtatctgctgctgctgctgctgctg

SEQ ID NO: 267 - DNA, 32 bp, synthetic construct
  misc_feature 1..32: note = Synthetic
  source 1..32: mol_type = other DNA
caggtaagtatctgctgctgctgctgctgctg

SEQ ID NO: 268 - DNA, 28 bp, synthetic construct
  misc_feature 1..28: note = Synthetic
  source 1..28: mol_type = other DNA
ctgctgctgctgctgctgctgcaggtaa

SEQ ID NO: 269 - DNA, 28 bp, synthetic construct
  misc_feature 1..28: note = Synthetic
  source 1..28: mol_type = other DNA
ctgctgctgctgctgctgctgaggtaag

SEQ ID NO: 270 - DNA, 28 bp, synthetic construct
  misc_feature 1..28: note = Synthetic
  source 1..28: mol_type = other DNA
ctgctgctgctgctgctgctgggtaagt

SEQ ID NO: 271 - DNA, 28 bp, synthetic construct
  misc_feature 1..28: note = Synthetic
  source 1..28: mol_type = other DNA
ctgctgctgctgctgctgctggtaagta

SEQ ID NO: 272 - DNA, 28 bp, synthetic construct
  misc_feature 1..28: note = Synthetic
  source 1..28: mol_type = other DNA
ctgctgctgctgctgctgctgtaagtat

SEQ ID NO: 273 - DNA, 29 bp, synthetic construct
  misc_feature 1..29: note = Synthetic
  source 1..29: mol_type = other DNA
ctgctgctgctgctgctgctgcaggtaag

SEQ ID NO: 274 - DNA, 29 bp, synthetic construct
  misc_feature 1..29: note = Synthetic
  source 1..29: mol_type = other DNA
ctgctgctgctgctgctgctgaggtaagt

SEQ ID NO: 275 - DNA, 29 bp, synthetic construct
  misc_feature 1..29: note = Synthetic
  source 1..29: mol_type = other DNA
ctgctgctgctgctgctgctgggtaagta

SEQ ID NO: 276 - DNA, 29 bp, synthetic construct
  misc_feature 1..29: note = Synthetic
  source 1..29: mol_type = other DNA
ctgctgctgctgctgctgctggtaagtat

SEQ ID NO: 277 - DNA, 30 bp, synthetic construct
  misc_feature 1..30: note = Synthetic
  source 1..30: mol_type = other DNA
ctgctgctgctgctgctgctgcaggtaagt

SEQ ID NO: 278 - DNA, 30 bp, synthetic construct
  misc_feature 1..30: note = Synthetic
  source 1..30: mol_type = other DNA
ctgctgctgctgctgctgctgaggtaagta

SEQ ID NO: 279 - DNA, 30 bp, synthetic construct
  misc_feature 1..30: note = Synthetic
  source 1..30: mol_type = other DNA
ctgctgctgctgctgctgctgggtaagtat

SEQ ID NO: 280 - DNA, 31 bp, synthetic construct
  misc_feature 1..31: note = Synthetic
  source 1..31: mol_type = other DNA
ctgctgctgctgctgctgctgcaggtaagta

SEQ ID NO: 281 - DNA, 31 bp, synthetic construct
  misc_feature 1..31: note = Synthetic
  source 1..31: mol_type = other DNA
ctgctgctgctgctgctgctgaggtaagtat

SEQ ID NO: 282 - DNA, 32 bp, synthetic construct
  misc_feature 1..32: note = Synthetic
  source 1..32: mol_type = other DNA
ctgctgctgctgctgctgctgcaggtaagtat

SEQ ID NO: 283 - RNA, 18 nt, synthetic construct
  misc_feature 1..18: note = Synthetic
  source 1..18: mol_type = other RNA
tgaggaactccgccgcct

SEQ ID NO: 284 - DNA, 18 bp, synthetic construct
  misc_feature 1..18: note = Synthetic
  source 1..18: mol_type = other DNA
aggcggcggagttcctca

SEQ ID NO: 285 - DNA, 25 bp, synthetic construct
  misc_feature 1..25: note = Synthetic
  source 1..25: mol_type = other DNA
caggtaaaggcggcggagttcctca

SEQ ID NO: 286 - DNA, 25 bp, synthetic construct
  misc_feature 1..25: note = Synthetic
  source 1..25: mol_type = other DNA
aggtaagaggcggcggagttcctca

SEQ ID NO: 287 - DNA, 25 bp, synthetic construct
  misc_feature 1..25: note = Synthetic
  source 1..25: mol_type = other DNA
ggtaagtaggcggcggagttcctca

SEQ ID NO: 288 - DNA, 25 bp, synthetic construct
  misc_feature 1..25: note = Synthetic
  source 1..25: mol_type = other DNA
gtaagtaaggcggcggagttcctca

SEQ ID NO: 289 - DNA, 25 bp, synthetic construct
  misc_feature 1..25: note = Synthetic
  source 1..25: mol_type = other DNA
taagtataggcggcggagttcctca

SEQ ID NO: 290 - DNA, 26 bp, synthetic construct
  misc_feature 1..26: note = Synthetic
  source 1..26: mol_type = other DNA
caggtaagaggcggcggagttcctca

SEQ ID NO: 291 - DNA, 26 bp, synthetic construct
  misc_feature 1..26: note = Synthetic
  source 1..26: mol_type = other DNA
aggtaagtaggcggcggagttcctca

SEQ ID NO: 292 - DNA, 26 bp, synthetic construct
  misc_feature 1..26: note = Synthetic
  source 1..26: mol_type = other DNA
ggtaagtaaggcggcggagttcctca

SEQ ID NO: 293 - DNA, 26 bp, synthetic construct
  misc_feature 1..26: note = Synthetic
  source 1..26: mol_type = other DNA
gtaagtataggcggcggagttcctca

SEQ ID NO: 294 - DNA, 27 bp, synthetic construct
  misc_feature 1..27: note = Synthetic
  source 1..27: mol_type = other DNA
caggtaagtaggcggcggagttcctca

SEQ ID NO: 295 - DNA, 27 bp, synthetic construct
  misc_feature 1..27: note = Synthetic
  source 1..27: mol_type = other DNA
aggtaagtaaggcggcggagttcctca

SEQ ID NO: 296 - DNA, 27 bp, synthetic construct
  misc_feature 1..27: note = Synthetic
  source 1..27: mol_type = other DNA
ggtaagtataggcggcggagttcctca

SEQ ID NO: 297 - DNA, 28 bp, synthetic construct
  misc_feature 1..28: note = Synthetic
  source 1..28: mol_type = other DNA
caggtaagtaaggcggcggagttcctca

SEQ ID NO: 298 - DNA, 28 bp, synthetic construct
  misc_feature 1..28: note = Synthetic
  source 1..28: mol_type = other DNA
aggtaagtataggcggcggagttcctca

SEQ ID NO: 299 - DNA, 29 bp, synthetic construct
  misc_feature 1..29: note = Synthetic
  source 1..29: mol_type = other DNA
caggtaagtataggcggcggagttcctca

SEQ ID NO: 300 - DNA, 25 bp, synthetic construct
  misc_feature 1..25: note = Synthetic
  source 1..25: mol_type = other DNA
aggcggcggagttcctcacaggtaa

SEQ ID NO: 301 - DNA, 25 bp, synthetic construct
  misc_feature 1..25: note = Synthetic
  source 1..25: mol_type = other DNA
aggcggcggagttcctcaaggtaag

SEQ ID NO: 302 - DNA, 25 bp, synthetic construct
  misc_feature 1..25: note = Synthetic
  source 1..25: mol_type = other DNA
aggcggcggagttcctcaggtaagt

SEQ ID NO: 303 - DNA, 25 bp, synthetic construct
  misc_feature 1..25: note = Synthetic
  source 1..25: mol_type = other DNA
aggcggcggagttcctcagtaagta

SEQ ID NO: 304 - DNA, 25 bp, synthetic construct
  misc_feature 1..25: note = Synthetic
  source 1..25: mol_type = other DNA
aggcggcggagttcctcataagtat

SEQ ID NO: 305 - DNA, 26 bp, synthetic construct
  misc_feature 1..26: note = Synthetic
  source 1..26: mol_type = other DNA
aggcggcggagttcctcacaggtaag

SEQ ID NO: 306 - DNA, 26 bp, synthetic construct
  misc_feature 1..26: note = Synthetic
  source 1..26: mol_type = other DNA
aggcggcggagttcctcaaggtaagt

SEQ ID NO: 307 - DNA, 26 bp, synthetic construct
  misc_feature 1..26: note = Synthetic
  source 1..26: mol_type = other DNA
aggcggcggagttcctcaggtaagta

SEQ ID NO: 308 - DNA, 26 bp, synthetic construct
  misc_feature 1..26: note = Synthetic
  source 1..26: mol_type = other DNA
aggcggcggagttcctcagtaagtat

SEQ ID NO: 309 - DNA, 27 bp, synthetic construct
  misc_feature 1..27: note = Synthetic
  source 1..27: mol_type = other DNA
aggcggcggagttcctcacaggtaagt

SEQ ID NO: 310 - DNA, 27 bp, synthetic construct
  misc_feature 1..27: note = Synthetic
  source 1..27: mol_type = other DNA
aggcggcggagttcctcaaggtaagta

SEQ ID NO: 311 - DNA, 27 bp, synthetic construct
  misc_feature 1..27: note = Synthetic
  source 1..27: mol_type = other DNA
aggcggcggagttcctcaggtaagtat

SEQ ID NO: 312 - DNA, 28 bp, synthetic construct
  misc_feature 1..28: note = Synthetic
  source 1..28: mol_type = other DNA
aggcggcggagttcctcacaggtaagta

SEQ ID NO: 313 - DNA, 28 bp, synthetic construct
  misc_feature 1..28: note = Synthetic
  source 1..28: mol_type = other DNA
aggcggcggagttcctcaaggtaagtat

SEQ ID NO: 314 - DNA, 29 bp, synthetic construct
  misc_feature 1..29: note = Synthetic
  source 1..29: mol_type = other DNA
aggcggcggagttcctcacaggtaagtat

SEQ ID NO: 315 - RNA, 25 nt, synthetic construct
  misc_feature 1..25: note = Synthetic
  source 1..25: mol_type = other RNA
caactgaaggtaaaatcaccacacg

SEQ ID NO: 316 - DNA, 25 bp, synthetic construct
  misc_feature 1..25: note = Synthetic
  source 1..25: mol_type = other DNA
cgtgtggtgattttaccttcagttg

SEQ ID NO: 317 - DNA, 32 bp, synthetic construct
  misc_feature 1..32: note = Synthetic
  source 1..32: mol_type = other DNA
caggtaacgtgtggtgattttaccttcagttg

SEQ ID NO: 318 - DNA, 32 bp, synthetic construct
  misc_feature 1..32: note = Synthetic
  source 1..32: mol_type = other DNA
aggtaagcgtgtggtgattttaccttcagttg

SEQ ID NO: 319 - DNA, 32 bp, synthetic construct
  misc_feature 1..32: note = Synthetic
  source 1..32: mol_type = other DNA
ggtaagtcgtgtggtgattttaccttcagttg

SEQ ID NO: 320 - DNA, 32 bp, synthetic construct
  misc_feature 1..32: note = Synthetic
  source 1..32: mol_type = other DNA
gtaagtacgtgtggtgattttaccttcagttg

SEQ ID NO: 321 - DNA, 32 bp, synthetic construct
  misc_feature 1..32: note = Synthetic
  source 1..32: mol_type = other DNA
taagtatcgtgtggtgattttaccttcagttg

SEQ ID NO: 322 - DNA, 33 bp, synthetic construct
  misc_feature 1..33: note = Synthetic
  source 1..33: mol_type = other DNA
caggtaagcgtgtggtgattttaccttcagttg

SEQ ID NO: 323 - DNA, 33 bp, synthetic construct
  misc_feature 1..33: note = Synthetic
  source 1..33: mol_type = other DNA
aggtaagtcgtgtggtgattttaccttcagttg

SEQ ID NO: 324 - DNA, 33 bp, synthetic construct
  misc_feature 1..33: note = Synthetic
  source 1..33: mol_type = other DNA
ggtaagtacgtgtggtgattttaccttcagttg

SEQ ID NO: 325 - DNA, 33 bp, synthetic construct
  misc_feature 1..33: note = Synthetic
  source 1..33: mol_type = other DNA
gtaagtatcgtgtggtgattttaccttcagttg

SEQ ID NO: 326 - DNA, 34 bp, synthetic construct
  misc_feature 1..34: note = Synthetic
  source 1..34: mol_type = other DNA
caggtaagtcgtgtggtgattttaccttcagttg

SEQ ID NO: 327 - DNA, 34 bp, synthetic construct
  misc_feature 1..34: note = Synthetic
  source 1..34: mol_type = other DNA
aggtaagtacgtgtggtgattttaccttcagttg

SEQ ID NO: 328 - DNA, 34 bp, synthetic construct
  misc_feature 1..34: note = Synthetic
  source 1..34: mol_type = other DNA
ggtaagtatcgtgtggtgattttaccttcagttg

SEQ ID NO: 329 - DNA, 35 bp, synthetic construct
  misc_feature 1..35: note = Synthetic
  source 1..35: mol_type = other DNA
caggtaagtacgtgtggtgattttaccttcagttg

SEQ ID NO: 330 - DNA, 35 bp, synthetic construct
  misc_feature 1..35: note = Synthetic
  source 1..35: mol_type = other DNA
aggtaagtatcgtgtggtgattttaccttcagttg

SEQ ID NO: 331 - DNA, 36 bp, synthetic construct
  misc_feature 1..36: note = Synthetic
  source 1..36: mol_type = other DNA
caggtaagtatcgtgtggtgattttaccttcagttg

SEQ ID NO: 332 - DNA, 32 bp, synthetic construct
  misc_feature 1..32: note = Synthetic
  source 1..32: mol_type = other DNA
cgtgtggtgattttaccttcagttgcaggtaa

SEQ ID NO: 333 - DNA, 32 bp, synthetic construct
  misc_feature 1..32: note = Synthetic
  source 1..32: mol_type = other DNA
cgtgtggtgattttaccttcagttgaggtaag

SEQ ID NO: 334 - DNA, 32 bp, synthetic construct
  misc_feature 1..32: note = Synthetic
  source 1..32: mol_type = other DNA
cgtgtggtgattttaccttcagttgggtaagt

SEQ ID NO: 335 - DNA, 32 bp, synthetic construct
  misc_feature 1..32: note = Synthetic
  source 1..32: mol_type = other DNA
cgtgtggtgattttaccttcagttggtaagta

SEQ ID NO: 336 - DNA, 32 bp, synthetic construct
  misc_feature 1..32: note = Synthetic
  source 1..32: mol_type = other DNA
cgtgtggtgattttaccttcagttgtaagtat

SEQ ID NO: 337 - DNA, 33 bp, synthetic construct
  misc_feature 1..33: note = Synthetic
  source 1..33: mol_type = other DNA
cgtgtggtgattttaccttcagttgcaggtaag

SEQ ID NO: 338 - DNA, 33 bp, synthetic construct
  misc_feature 1..33: note = Synthetic
  source 1..33: mol_type = other DNA
cgtgtggtgattttaccttcagttgaggtaagt

SEQ ID NO: 339 - DNA, 33 bp, synthetic construct
  misc_feature 1..33: note = Synthetic
  source 1..33: mol_type = other DNA
cgtgtggtgattttaccttcagttgggtaagta

SEQ ID NO: 340 - DNA, 33 bp, synthetic construct
  misc_feature 1..33: note = Synthetic
  source 1..33: mol_type = other DNA
cgtgtggtgattttaccttcagttggtaagtat

SEQ ID NO: 341 - DNA, 34 bp, synthetic construct
  misc_feature 1..34: note = Synthetic
  source 1..34: mol_type = other DNA
cgtgtggtgattttaccttcagttgcaggtaagt

SEQ ID NO: 342 - DNA, 34 bp, synthetic construct
  misc_feature 1..34: note = Synthetic
  source 1..34: mol_type = other DNA
cgtgtggtgattttaccttcagttgaggtaagta

SEQ ID NO: 343 - DNA, 34 bp, synthetic construct
  misc_feature 1..34: note = Synthetic
  source 1..34: mol_type = other DNA
cgtgtggtgattttaccttcagttgggtaagtat

SEQ ID NO: 344 - DNA, 35 bp, synthetic construct
  misc_feature 1..35: note = Synthetic
  source 1..35: mol_type = other DNA
cgtgtggtgattttaccttcagttgcaggtaagta

SEQ ID NO: 345 - DNA, 35 bp, synthetic construct
  misc_feature 1..35: note = Synthetic
  source 1..35: mol_type = other DNA
cgtgtggtgattttaccttcagttgaggtaagtat

SEQ ID NO: 346 - DNA, 36 bp, synthetic construct
  misc_feature 1..36: note = Synthetic
  source 1..36: mol_type = other DNA
cgtgtggtgattttaccttcagttgcaggtaagtat

SEQ ID NO: 347-353 - skipped (7 entries)

SEQ ID NO: 354 - DNA, 25 bp, synthetic construct
  misc_feature 1..25: note = Synthetic
  source 1..25: mol_type = other DNA
tgtgagcaccttccttctttaggta

SEQ ID NO: 355 - DNA, 25 bp, synthetic construct
  misc_feature 1..25: note = Synthetic
  source 1..25: mol_type = other DNA
tgtgagcaccttccttctttgtaag

SEQ ID NO: 356 - DNA, 25 bp, synthetic construct
  misc_feature 1..25: note = Synthetic
  source 1..25: mol_type = other DNA
tgtgagcaccttccttctttagtat

SEQ ID NO: 357 - RNA, 14 nt, synthetic construct
  misc_feature 1..14: note = Synthetic
  source 1..14: mol_type = other RNA
atacttacctggca